(19) United States
(12) Patent Application Publication
(10) Pub. No.: US 2022/0401428 A1
(43) Pub. Date: Dec. 22, 2022

(54) Method for Treatment of Alzheimer's Disease

(71) Applicant: University of Kentucky Research Foundation, Lexington, KY (US)

(72) Inventor: Florin Despa, Lexington, KY (US)

(21) Appl. No.: 17/764,073

(22) PCT Filed: Oct. 1, 2020
(86) PCT No.: PCT/US20/53787

§ 371 Date: Mar. 25, 2022

Related U.S. Application Data
(60) Provisional application No. 62/908,937, filed on Oct. 1, 2019.

(51) Int. Cl. A61K 31/4468; C12N 15/113; A61P 25/28

(52) U.S. Cl. CPC A61K 31/4468; C12N 15/1138; A61P 25/28; C12N 2310/141

(57) ABSTRACT

The presently-disclosed subject matter generally relates to methods for treating a subject with Alzheimer's Disease, microhemorrhages, and neurological deficits. The presently-disclosed subject matter also relates to methods for treating a subject with Alzheimer's Disease, microhemorrhages, and neurological deficits with a composition that increases epoxyeicosatrienoic acids. The presently-disclosed subject matter further relates to a method of treating or preventing Alzheimer's Disease comprising administering an agent that increases vascular LRP1 expression.

### RELATED APPLICATIONS

This application claims priority from U.S. Provisional Patent Application No. 62/908,937 filed on Oct. 1, 2019 the entire disclosure of which is incorporated herein by this reference.

### GOVERNMENT INTEREST

This invention was made with government support under grant number AG053999, awarded by the National Institutes of Health. The government has certain rights in the invention.

### SEQUENCE LISTING

This application contains a sequence listing submitted in accordance with 37 C.F.R. 1.821, named 13177N 2025US DESPA sequence listing.txt, created on Oct. 1, 2020, having a size of 2,146 bytes, which is incorporated herein by this reference.

### TECHNICAL FIELD

The present invention relates to methods for treating a subject with Alzheimer's Disease, microhemorrhages, and neurological deficits. Some aspects of the present invention relates to methods for treating a subject with Alzheimer's Disease, microhemorrhages, and neurological deficits with a composition that increases epoxyeicosatrienoic acids. The present invention also relates to a method of treating or preventing Alzheimer's Disease comprising administering an agent that increases vascular LRP1 expression.

### BACKGROUND

Alzheimer's disease (AD) is caused by an imbalance between production and clearance of aggregation-prone amyloid-β (Aβ) species linked to a genetic predisposition (i.e., familial AD; fAD) or pathologic aging processes (i.e., sporadic AD; sAD)¹⁻⁴. Mechanisms underlying pathologic aging remain unknown. The endocrine hormone amylin modulates brain amyloid composition in both sporadic and familial forms of AD and that pancreatic overexpression of human amylin in a rat model of AD (rat amylin is non-amyloidogenic⁵) accelerates pathologic aging, whereas genetic or pharmacologic suppression of amylin expression is protective. By staining and imaging of amylin and Aβ in brain tissues from humans with and without fAD, AD rats expressing human amylin and AD rats expressing non-amyloidogenic rat amylin, amylin accumulated in small vessels, paired with Aβ in neuritic plaques and also formed independent neuritic plaques and space-filling lesion within neurons, independent of tau pathology. In cerebrospinal fluid (CSF), amylin and Aβ₄₂ levels were inversely correlated with age in sAD, mild cognitive impairment and normal aging. A synergy between increased CSF amylin levels and AD pathology was seen in AD rats expressing human amylin. The mechanisms underlying accelerated aging and behavior changes in AD rats expressing human amylin involved hypoxic-ischemic brain injury leading to neurodegeneration. These pathological processes were reduced by pharmacological activation of protective mechanisms within endothelial cells, which lowered amylin deposition in brain capillaries. Genetic suppression of amylin in AD rats increased body weight, consistent with amylin's action as a satiety hormone⁶, but also reduced neurologic deficits. The results show that amylin dyshomeostasis is a causative mechanism of pathological aging and suggest that drugs reducing amylin deposition in brain capillaries or preventing amylin from interacting with Aβ pathology could provide benefit in AD.

Amylin is co-synthesized with insulin by pancreatic β-cells⁷ and normally crosses the blood-brain barrier participating in the central regulation of satiety⁶. It is degraded by the insulin degrading enzyme⁸, like insulin and Aβ. In patients with type-2 diabetes, amylin forms pancreatic amyloid⁷ (FIG. 1A) causing apoptosis and depletion of β-cell mass⁹. Amylin deposition was detected also in failing human hearts¹⁰ and brains of individuals with sAD¹¹⁻¹⁷ (reviewed in Ref 18). Whether amylin dyshomeostasis affects the brain in fAD remains unknown.

Cerebral small vessel diseases are significant contributors to vascular cognitive impairment and dementia (VCID)¹ and a common pathological finding in the brains of individuals with Alzheimer's disease (AD)²⁻⁴. Mechanisms underlying small vessel-type dysfunction include cerebral amyloid angiopathy (CAA) caused by vascular deposition of amyloid β (Aβ) protein, arteriolosclerosis associated with aging, hypertension, and cardiovascular risk factors'. In addition, accumulating evidence from clinical studies demonstrates that obesity, insulin resistance and diabetes are strong risk factors for cerebral microvascular dysfunction⁶ and the sporadic form of AD⁷⁻⁹. Correcting hyperglycemia, the hallmark of diabetic states, is not entirely effective at reestablishing vascular endothelial function^{10,11} nor reducing cognitive decline¹²⁻¹⁵. Thus, blood glucose levels per se may not be the correct target for cerebrovascular disease and cognitive decline risk reduction.

Recent reports from multiple laboratories¹⁶⁻²¹ show that vascular lesions and Aβ plaques in the brains of individuals with AD have abundant deposits of amylin, a ˜4 kDa hormone synthesized and co-secreted with insulin by pancreatic β-cells²². Amylin normally crosses the blood-brain barrier (BBB)²³ and participates in the central regulation of satiation²⁴, but it also forms pancreatic amyloid in patients with type-2 diabetes²². In patients with type-2 diabetes, aggregated amylin accumulates in the cardiovascular system (systemic amylin dyshomeostasis)²⁵⁻²⁷ and is associated with microcirculatory disturbances and activation of hypoxia signaling in kidneys through attachment to red blood cells²⁷. To uncover cerebral effects associated with systemic pancreatic amylin dyshomeostasis, Rats that express human amylin in the pancreatic β-cells^{18,28}, as amylin from rodents is non-amyloidogenic²⁹ and less prone to deposition in blood vessels¹⁸ were previously studied. Human amylin-expressing rats slowly accumulate aggregated amylin in the brain microvasculature with aging (>12-month old rats) leading to microhemorrhages¹⁸ and late-onset behavioral changes^{18,28} that are similar to those in AD rat models. At the cellular and molecular levels, accumulation of aggregated amylin in brain capillaries is associated with astrocyte activation, neuroinflammation and oxidative stress^{18,28}.

Cell responses to stress conditions involve reprograming gene expression through non-coding RNAs such as microRNAs (miRNAs)³⁰. They inhibit protein synthesis by suppressing the translation of protein coding genes or by degrading the mRNA³⁰. Paralog miRNAs miR-103 and miR-107 have previously been shown to be dysregulated in AD³¹. These miRNAs also appear to mediate stress-suppressed translation of the low-density lipoprotein receptor-related protein 1 (LRP1)³², an apolipoprotein E (APOE) receptor that binds and internalizes soluble Aβ at the abluminal side of the BBB³³⁻³⁵. Because amylin deposition in the brain microvasculature affects vascular endothelial cells (ECs)¹⁸, amylin stress dysregulates miR-103/107 impairing LRP1 synthesis and Aβ efflux across the BBB antagomirs against miR-103/107 modulate the amylin-mediated stress effect on LRP1.

To determine whether amylin deposition in the brain microvasculature is associated with impaired Aβ efflux, amylin-Aβ interaction in the human brain microvasculature was explored and carried out in vivo analyses of Aβ efflux across the BBB in rats that express amyloid-forming human amylin in pancreatic β-cells versus littermates that express non-amyloidogenic rat amylin. To further define the mechanism, an in vitro BBB model of Aβ transcytosis was used in which the EC monolayer was exposed to amylin-mediated stress; antisense microRNAs were used in an attempt to rescue endothelial LRP1 expression. The instant results provide a basis for targeting amylin-mediated cellular pathways at the blood-brain interface to reduce or prevent AD pathology.

### SUMMARY

The presently-disclosed subject matter meets some or all of the above-identified needs, as will become evident to those of ordinary skill in the art after a study of information provided in this document.

This Summary describes several embodiments of the presently-disclosed subject matter, and in many cases lists variations and permutations of these embodiments. This Summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently-disclosed subject matter, whether listed in this Summary or not. To avoid excessive repetition, this Summary does not list or suggest all possible combinations of such features.

One embodiment of the present invention is a method of reducing the amount of systemic amylin comprising: administering to a subject in need thereof an effective amount of a composition that increases epoxyeicosatrienoic acids. In some embodiments of the present invention, the composition that increases epoxyeicosatrienoic acids is a soluble epoxide hydrolase inhibitor. In further embodiments of the present invention, the composition that increases epoxyeicosatrienoic acids is a soluble epoxide hydrolase inhibitor. In some embodiments, the soluble epoxide hydrolase inhibitor is 1-(1-propanoylpiperidin-4-yl)-3-[4-(trifluoromethoxy)phenyl]urea (TPPU). In other embodiments of the invention, TPPU is administered orally or intravenously. In further embodiments of the present invention, the subject is administered a dose of about 20 micrograms per kilogram TPPU.

Other embodiments of the present invention include a method of treating a subject diagnosed with a neurological disease or deficiency, said method comprising: identifying a subject diagnosed with a neurological disease or deficiency and administering an effective amount of a composition that increases epoxyeicosatrienoic acids. In further embodiments of the present invention, the composition that increases epoxyeicosatrienoic acids is a soluble epoxide hydrolase inhibitor. In some embodiments, the soluble epoxide hydrolase inhibitor is 1-(1-propanoylpiperidin-4-yl)-344-(trifluoromethoxy)phenyllurea (TPPU). In other embodiments of the invention, TPPU is administered orally or intravenously. In further embodiments of the present invention, the subject is administered a dose of about 20 micrograms per kilogram TPPU. In some embodiments of the instant invention, the neurological disease or deficiency is selected from: hypoxic-ischemic brain injury, Alzheimer's Disease, neurological deficits, brain microhemorrhages, or axonal degeneration.

Another embodiment of the present invention includes a method of treating Alzheimer's Disease comprising: administering an agent that increases LRP1 expression to a subject in need thereof. In some embodiments of the present invention, the upregulators of LRP1 are antagomirs against miRNAs. In further embodiments of the present invention, the administration occurs for at least 12 hours. In some embodiments of the present invention, the miRNA is miR-103 agcagcauuguacagggcuauga (SEQ ID NO: 5). In other embodiments of the present invention, the miRNA is miR-107 agcuucuuuacaguguugccuugu (SEQ ID NO: 6). In some embodiments of the present invention, the miRNA is administered to the subject at a concentration of about 100 nM. In further embodiments of the present invention, and further comprising administering both miRNA is miR-103 agcagcauuguacagggcuauga (SEQ ID No: 5) and miR-107 agcuucuuuacaguguugccuugu (SEQ ID NO: 6) to the subject.

### BRIEF DESCRIPTION OF THE DRAWINGS

The presently-disclosed subject matter will be better understood, and features, aspects and advantages other than those set forth above will become apparent when consideration is given to the following detailed description thereof. Such detailed description makes reference to the following drawings, wherein:

FIG. 1A shows the pancreatic hormone amylin modulates brain amyloid composition and pathology distribution in PSEN1 and APP mutation carriers. Schematic. Amylin is a pancreatic hormone that participates in the central regulation of satiety (blue). In patients with type-2 diabetes, amylin forms pancreatic amyloid (brown). Scale bar, 100 μm. In patients with AD, amylin modulates brain amyloid and contributes to small vessel ischemic disease (SVID) (magenta).

FIG. 1B shows the pancreatic hormone amylin modulates brain amyloid composition and pathology distribution in PSEN1 and APP mutation carriers. Schematic. Amylin levels in the homogenates of human temporal cortex from PSEN1 and APP mutation carriers (fAD; n=18) and from cognitively normal individuals (CN; n=23.) were measured using an amylin ELISA.Scale bar, 50 μm. Data are means+SEM

FIG. 1C shows the pancreatic hormone amylin modulates brain amyloid composition and pathology distribution in PSEN 1 and APP mutation carriers. A combination of anti-amylin antibody with anti-Aβ antibody generated immunoreactivity signals in fAD temporal cortex sections. Amylin formed homologous neuritic plaques (c, d; arrows), intraneural deposits (c; arrow heads). Representative images are from fAD brains with mutation in PSEN1 intron 4. Scale bar, 50 μm

FIG. 1D shows the pancreatic hormone amylin modulates brain amyloid composition and pathology distribution in PSEN1 and APP mutation carriers. A combination of anti-amylin antibody with anti-Aβ antibody generated immunoreactivity signals in fAD temporal cortex sections. Amylin formed homologous neuritic plaques Amylin accumulated in small blood vessels. Representative images are from fAD brains with mutation in PSEN1 A434T and T291A. Scale bar, 50 μm.

FIG. 1E shows the pancreatic hormone amylin modulates brain amyloid composition and pathology distribution in PSEN1 and APP mutation carriers. A combination of anti-amylin antibody with anti-Aβ antibody generated immunoreactivity signals in fAD temporal cortex sections. Amylin formed heterologous deposits in which amylin and Aβ displayed layered structures that have an amylin-positive core. Representative images are from fAD brains with mutation in PSEN1 R2781.

FIG. 1F shows the pancreatic hormone amylin modulates brain amyloid composition and pathology distribution in PSEN1 and APP mutation carriers. A combination of anti-amylin antibody with anti-β antibody generated immunoreactivity signals in fAD temporal cortex sections. Amylin formed heterologous deposits in which amylin and Aβ displayed layered structures that have tightly mixed molecular structures. Scale bar, 50 μm.

FIG. 1G shows the pancreatic hormone amylin modulates brain amyloid composition and pathology distribution in PSEN1 and APP mutation carriers. A combination of anti-amylin antibody with anti-Aβ antibody generated immunoreactivity signals in fAD temporal cortex sections. Amylin accumulated in small blood vessels. Representative images are from fAD brains with mutation in PSEN1 R2781.

FIG. 1H shows the pancreatic hormone amylin modulates brain amyloid composition and pathology distribution in PSEN1 and APP mutation carriers. A combination of anti-amylin antibody with anti-Aβ antibody generated immunoreactivity signals in fAD temporal cortex sections. Amylin accumulated in small blood vessels. Representative images are from fAD brains with mutation in PSEN1 E184D. Scale bar, 100 μm.

FIG. 1I shows the pancreatic hormone amylin modulates brain amyloid composition and pathology distribution in PSEN1 and APP mutation carriers. Estimated amylin-positive vs Aβ-positive areas in the grey matter and white matter regions of fAD brains (n=27). Data are presented as area percentage. Scale bar, 50 μm. Data are means+SEM.

FIG. 1J shows the pancreatic hormone amylin modulates brain amyloid composition and pathology distribution in PSEN1 and APP mutation carriers. Confocal microscopy analysis of brain tissue from a patient with fAD (PSEN1 S132A) triple stained with Thioflavin S (ThioS, green), anti-amylin antibody (red) and anti-Aβ antibody (magenta) showing a neuritic plaque in which amylin formed the amyloid core (overlay). Scale bar, 10 μm.

FIG. 1K shows the pancreatic hormone amylin modulates brain amyloid composition and pathology distribution in PSEN1 and APP mutation carriers. The relationship between CSF amylin and Aβ42 levels in patients with sAD (magenta; n=36). Individuals with mild cognitive impairment (MCI; red; n=70) and CSF Aβ42<680 ng/L. The relationship between CSF amylin and Aβ42 levels in patients with sAD (magenta; n=36). correlation analysis; P<0.05 *, P<0.0001 ****; by two-tailed, unpaired Student's t test.

FIG. 1L shows the pancreatic hormone amylin modulates brain amyloid composition and pathology distribution in PSEN1 and APP mutation carriers. Individuals with mild cognitive impairment (MCI; red; n=70) and CSF Aβ42<680 ng/L. correlation analysis; P<0.05 *, P<0.0001 ****; by two-tailed, unpaired Student's t test.

FIG. 1M shows the pancreatic hormone amylin modulates brain amyloid composition and pathology distribution in PSEN1 and APP mutation carriers. In cognitively normal individuals (CN; green; n=83); green and black dots tagged samples from individuals with CSF Aβ42<680 ng/L and CSF Aβ42≥680 ng/L, respectively. correlation analysis; P<0.05 *, P<0.0001 ****; by two-tailed, unpaired Student's t test.

FIG. 1N shows the pancreatic hormone amylin modulates brain amyloid composition and pathology distribution in PSEN 1 and APP mutation carriers. The relationship between CSF amylin levels and age in same individuals as in FIGs. K-M. AD groups. correlation analysis; P<0.05 *, P<0.0001 ****; by two-tailed, unpaired Student's t test.

FIG. 1O shows the pancreatic hormone amylin modulates brain amyloid composition and pathology distribution in PSEN 1 and APP mutation carriers. The relationship between CSF amylin levels and age in same individuals as in FIGs. K-M. MCI groups. correlation analysis; P<0.05 *, P<0.0001 ****; by two-tailed, unpaired Student's t test.

FIG. 1P shows the pancreatic hormone amylin modulates brain amyloid composition and pathology distribution in PSEN1 and APP mutation carriers. The relationship between CSF amylin levels and age in same individuals as in FIGs. K-M. CN groups. correlation analysis; P<0.05 *, P<0.0001 ****; by two-tailed, unpaired Student's t test.

FIG. 1Q shows Consecutive temporal cortex sections from a patient with fAD (PSEN1 R278I) stained for amyloid with Congo Red.

FIG. 1R shows Consecutive temporal cortex sections from a patient with fAD immunohistochemistry with anti-amylin antibody.

FIG. 1S shows Consecutive temporal cortex sections from a patient with fAD, anti-Aβ antibody.

FIG. 1T shows Consecutive temporal cortex sections from a patient with fAD, a combination of anti-amylin and anti-Aβ antibodies.

FIG. 2A shows pancreatic overexpression of human amylin in AD rats accelerates aging and behavior deficits; genetic suppression of amylin expression is protective. Average z-scores of behavior tests (see also FIGS. 6a-f) in AD rats and AD rats expressing human amylin (ADHIP rats) (n=10 rats/group) at 8 months (8 M), 12 months (12 M) and 16 months (16 M) of age. Scale bar, 50 μm. Data are means±SEM P<0.05 *, P<0.01 **, P<0.0001 ****; by repeated measures ANOVA with Bonferroni post-hoc.

FIG. 2B shows pancreatic overexpression of human amylin in AD rats accelerates aging and behavior deficits; genetic suppression of amylin expression is protective. Average z-scores of behavior tests (see also FIGS. 6h-j) in AD rats and AD rats with deleted amylin genes (AD-AKO rats) at 12 M and 16 M of age (n=6 rats/group). Scale bar, 50 μm. Data are means±SEM ; P<0.05 *, P<0.01 **, P<0.0001 ****; by repeated measures ANOVA with Bonferroni post-hoc.

FIG. 2C shows pancreatic overexpression of human amylin in AD rats accelerates aging and behavior deficits; genetic suppression of amylin expression is protective. Representative images comparing ADHIP AD rats at 12 M and 16 M of age. Scale bar, 50 μm.

FIG. 2D shows pancreatic overexpression of human amylin in AD rats accelerates aging and behavior deficits; genetic suppression of amylin expression is protective. Representative images comparing ADHIP AD-AKO rats at 12 M and 16 M of age.

FIG. 2E shows pancreatic overexpression of human amylin in AD rats accelerates aging and behavior deficits; genetic suppression of amylin expression is protective. Representative images comparing AD-AKO rats at 12 M and 16 M of age. Scale bar, 50 μm.

FIG. 2F shows pancreatic overexpression of human amylin in AD rats accelerates aging and behavior deficits; genetic suppression of amylin expression is protective. ADHIP rats have dull coats, kyphosis and poor grooming at 16 M of age. Two slices (slice 3 and 5 out of 7 consecutive slices, lmm apart) of coronal T₂-weighted magnetic resonance (MR) images comparing the brains of ADHIP and AD rats (16 M old, n=7 rats/group). Hyperintensity areas in the temporal horns of the hippocampus (arrow heads) and the lateral ventricles (arrows) of ADHIP rats reflect extracellular fluid accumulation. Scale bar, 50 μm.

FIG. 2G shows pancreatic overexpression of human amylin in AD rats accelerates aging and behavior deficits; genetic suppression of amylin expression is protective. Volumes of ventricular hyperintesnsity in the brains of ADHIP and AD rats (n=7 rats/group) computed from coronal T₂-weighted MR images; 1 image pixel area=0.024 mm². Data are means±SEM; P<0.05 *, P<0.01 **, P<0.0001 ****; by repeated measures ANOVA with two-tailed, unpaired Student's t test.

FIG. 2H shows pancreatic overexpression of human amylin in AD rats accelerates aging and behavior deficits; genetic suppression of amylin expression is protective. Cross-sectional analyses of CSF total Aβ levels vs age in ADHIP and AD rats (n=4 rats/group). Data are means±SEM; P<0.05 *, P<0.01 **, P<0.0001 ****; two-tailed, unpaired Student's t test.

FIG. 3A shows amylin dyshomeostasis induces hypoxic-ischemic brain injury leading to axonal degeneration. Plasma erythropoietin (EPO) levels in 16 M old ADHIP and AD rats (n=7 rats/group). Data are means±SEM; P<0.05 *, P<0.01 **; by two-tailed, unpaired Student's t test.

FIG. 3B shows amylin dyshomeostasis induces hypoxic-ischemic brain injury leading to axonal degeneration. Relative mitochondrial DNA content from brains of 16 M old ADHIP and AD rats (n=7 rats/group). Data are means±SEM; P<0.05 *, P<0.01 **; by two-tailed, unpaired Student's t test.

FIG. 3C shows amylin dyshomeostasis induces hypoxic-ischemic brain injury leading to axonal degeneration. The protein levels of hypoxia inducible factors 1α and 260 (HIF-1α; HIF-2α) in brain capillary lysates from 16 M old ADHIP and AD rats (n=7 rats/group). HIF-1α and HIF-2α levels were normalized to the total protein input. Data are means±SEM; P<0.05 *, P<0.01 **; by two-tailed, unpaired Student's t test.

FIG. 3D shows amylin dyshomeostasis induces hypoxic-ischemic brain injury leading to axonal degeneration. The protein levels of vascular cell adhesion molecule 1 (VCAM-1) in brain capillary lysates from 16 M old ADHIP and AD rats (n=8 rats/group). Data are means±SEM; P<0.05 *, P<0.01 **; by two-tailed, unpaired Student's t test.

FIG. 3E shows amylin dyshomeostasis induces hypoxic-ischemic brain injury leading to axonal degeneration. Representative images of histological analysis of microglia activation marker (Ionized calcium binding adaptor molecule 1, Iba-1) in brain sections from ADHIP and AD rats (n=5 rats/group). Scale bar, 50 μm. (Cor—cortex; Hipp—hippocampus; Tha—thalamus; Htha—hypothalamus; CC—corpus callosum; LV—lateral ventricle area)

FIG. 3F shows amylin dyshomeostasis induces hypoxic-ischemic brain injury leading to axonal degeneration. Representative images of histological analysis of microglia activation marker (Ionized calcium binding adaptor molecule 1, Iba-1) in brain sections from ADHIP and AD rats (n=5 rats/group). Scale bar, 50 μm. (Cor—cortex; Hipp—hippocampus; Tha—thalamus; Htha—hypothalamus; CC—corpus callosum; LV—lateral ventricle area)

FIG. 3G shows amylin dyshomeostasis induces hypoxic-ischemic brain injury leading to axonal degeneration. Representative images of histological analysis of macrophage marker (Cluster of differentiation 68, CD68) in brain sections from ADHIP and AD rats (n=5 rats/group). Scale bar, 50 μm; P<0.05 *, P<0.01 **; by two-tailed, unpaired Student's t test. (Cor—cortex; Hipp—hippocampus; Tha—thalamus; Htha—hypothalamus; CC—corpus callosum; LV—lateral ventricle area)

FIG. 3H shows amylin dyshomeostasis induces hypoxic-ischemic brain injury leading to axonal degeneration. Representative images of histological analysis of macrophage marker (Cluster of differentiation 68, CD68) in brain sections from ADHIP and AD rats (n=5 rats/group). Scale bar, 50 μm, (Cor—cortex; Hipp—hippocampus; Tha—thalamus; Htha—hypothalamus; CC—corpus callosum; LV—lateral ventricle area). Data are means±SEM; P<0.05 *, P<0.01 **; by two-tailed, unpaired Student's t test.

FIG. 3I shows amylin dyshomeostasis induces hypoxic-ischemic brain injury leading to axonal degeneration. Representative images of histological analysis of Luxol fast blue (LFB) in brain sections from ADHIP and AD rats (n=5 rats/group). Scale bar, 50 μm. (Cor—cortex; Hipp—hippocampus; Tha—thalamus; Htha—hypothalamus; CC—corpus callosum; LV—lateral ventricle area). P<0.05 *, P<0.01 **; by two-tailed, unpaired Student's t test.

FIG. 3J shows amylin dyshomeostasis induces hypoxic-ischemic brain injury leading to axonal degeneration. Representative images of histological analysis of Luxol fast blue (LFB) in brain sections from ADHIP and AD rats (n=5 rats/group). (Cor—cortex; Hipp—hippocampus; Tha—thalamus; Htha—hypothalamus; CC—corpus callosum; LV—lateral ventricle area). Data are means±SEM; P<0.05 *, P<0.01 **; by two-tailed, unpaired Student's t test.

FIG. 3K shows amylin dyshomeostasis induces hypoxic-ischemic brain injury leading to axonal degeneration. Representative images of histological analysis of myelin basic protein (MBP) in brain sections from ADHIP and AD rats (n=5 rats/group). Scale bar, 50 μm, (Cor—cortex; Hipp—hippocampus; Tha—thalamus; Htha—hypothalamus; CC—corpus callosum; LV—lateral ventricle area); P<0.05 *, P<0.01 **; by two-tailed, unpaired Student's t test.

FIG. 3L shows amylin dyshomeostasis induces hypoxic-ischemic brain injury leading to axonal degeneration. Representative images of histological analysis of myelin basic protein (MBP) in brain sections from ADHIP and AD rats (n=5 rats/group). (Cor—cortex; Hipp—hippocampus; Tha—thalamus; Htha—hypothalamus; CC—corpus callosum; LV—lateral ventricle area). Data are means±SEM; P<0.05 *, P<0.01 **; by two-tailed, unpaired Student's t test.

FIG. 3M shows amylin dyshomeostasis induces hypoxic-ischemic brain injury leading to axonal degeneration. Schematic of proposed molecular mechanism underlying the deleterious effects of amylin dyshomeostasis on the brain small vessels and white matter: Amylin deposition in small vessels provokes microhemorrhages and impaired transport of O₂ and nutrients across the capillary endothelium leading to activation of hypoxia signaling pathways, remodeling of mitochondria and axonal degeneration.

FIG. 4A shows pharmacologically ameliorated amylin dyshomeostasis improves functionality of human amylin-expressing AD rats. Diagram of pharmacological interventions to ameliorate early stage and late-stage amylin dyshomeostasis in ADHIP rats using a soluble epoxide hydrolase inhibitor (sEHi).

FIG. 4B shows pharmacologically ameliorated amylin dyshomeostasis improves functionality of human amylin-expressing AD rats. Average z-scores of behavioral tests (see also FIG. 8) in the sEHi late-stage treated ADHIP rats (T) vs control (untreated; UT) rats, before treatment (16 M) and after treatment (18 M) (n=6 rats/group).

FIG. 4C shows pharmacologically ameliorated amylin dyshomeostasis improves functionality of human amylin-expressing AD rats. Average z-scores of behavioral tests (see also FIG. 8) in the early-stage (ES) treated ADHIP rats (T-ES) vs control (untreated; UT) rats were 12 M of age at the beginning of the treatment and 16 M of age at the end of treatment (n=5 rats/group) (T-ES).

FIG. 4D shows pharmacologically ameliorated amylin dyshomeostasis improves functionality of human amylin-expressing AD rats. The protein levels of HIF-1α and HIF-2α.

FIG. 4E shows pharmacologically ameliorated amylin dyshomeostasis improves functionality of human amylin-expressing AD rats. The protein levels of Arg-1 and Arg-2.

FIG. 4F shows pharmacologically ameliorated amylin dyshomeostasis improves functionality of human amylin-expressing AD rats. The protein levels of of arginase activity in brain capillary lysates from T vs UT rats (n=6 rats/group). HIF-1α, HIF-2α, Arg-1, Arg-2 and arginase activity levels were normalized to the total protein input.

FIG. 4G shows pharmacologically ameliorated amylin dyshomeostasis improves functionality of human amylin-expressing AD rats. Relative mitochondrial DNA content from brains of T vs UT rats (n=6 rats/group).

FIG. 4H shows pharmacologically ameliorated amylin dyshomeostasis improves functionality of human amylin-expressing AD rats. Representative images and analysis for amylin deposition in brain capillaries from UT vs T rats (n=3/group). Scale bar, 50 μm.

FIG. 4I shows pharmacologically ameliorated amylin dyshomeostasis improves functionality of human amylin-expressing AD rats. Representative images and analysis for amylin deposition in brain capillaries from UT vs T rats (n=3/group). Scale bar, 50 μm.

FIG. 4J shows pharmacologically ameliorated amylin dyshomeostasis improves functionality of human amylin-expressing AD rats. Representative images and analysis for amylin deposition in brain capillaries from UT vs T rats (n=3/group). Scale bar, 50 μm.

FIG. 4K shows pharmacologically ameliorated amylin dyshomeostasis improves functionality of human amylin-expressing AD rats. Representative images and analysis for brain microhemorrhages stained with Prussian blue dye in brains of T vs UT rats (n=3 rats/group). Representative images are from the brain cortex. Scale bar, 20 μm.

FIG. 4L shows pharmacologically ameliorated amylin dyshomeostasis improves functionality of human amylin-expressing AD rats. Representative images and analysis for brain microhemorrhages stained with Prussian blue dye in brains of T vs UT rats (n=3 rats/group). Representative images are from the brain cortex. Scale bar, 20 μm.

FIG. 4M shows pharmacologically ameliorated amylin dyshomeostasis improves functionality of human amylin-expressing AD rats. Representative images and analysis for brain microhemorrhages stained with Prussian blue dye in brains of T vs UT rats (n=3 rats/group). Representative images are from the brain cortex. Scale bar, 20 μm.

FIG. 4N shows pharmacologically ameliorated amylin dyshomeostasis improves functionality of human amylin-expressing AD rats. Schematic. Targeting amylin dyshomeostasis by pharmacological agent (sEHI) in ADHIP rats reduced capillary amylin deposition, which ameliorated hypoxic-ischemic brain injury and axonal degeneration (see FIG. 8) resulting in attenuation of functional decline.

FIG. 5A shows Amylin levels in homogenates of human temporal cortex tissues from PSEN1 mutation carriers (n=11) and from APP mutation carriers (n=7) were measured using an amylin ELISA. Scale bar, 200 μm. Data are means±SEM (a); P<0.05 *, P<0.01 **, P<0.001 ***, P<0.0001 ****; by two-tailed, unpaired Student's t test.

FIG. 5B shows Amylin levels in homogenates of human temporal cortex tissues from PSEN1 mutation carriers (n=11) and from APP mutation carriers (n=7) were measured using an amylin ELISA. Estimated partition of amylin and Aβ in the grey matter plaques (left panel). Analysis is based on the immunohistochemistry staining with anti-amylin and anti-Aβ antibodies. In the right panel, the partition of amylin and Aβ immunoreactivity signals is shown within mixed amylin-Aβ plaques, in the grey matterScale bar, 200 μm. Data are means±SEM (a); P<0.05 *, P<0.01 **, P<0.001 ***, P<0.0001 ****; by two-tailed, unpaired Student's t test (a, b).

FIG. 5C shows Amylin levels in homogenates of human temporal cortex tissues from PSEN1 mutation carriers (n=11) and from APP mutation carriers (n=7) were measured using an amylin ELISA. Confocal microscopy analysis of brain tissue from a patient with fAD (PSEN1 intron 4) double stained with anti-amylin antibody (green) and anti-p-tau antibody (red). Nuclei are stained blue. Scale bar, 10 μm.

FIG. 5D shows Amylin levels in homogenates of human temporal cortex tissues from PSEN1 mutation carriers (n=11) and from APP mutation carriers (n=7) were measured using an amylin ELISA. Representative image showing typical distribution of amylin and Aβ immunoreactivities in grey matter and white matter regions of a fAD brain. Scale bar, 200 μm.

FIG. 5E shows Amylin levels in homogenates of human temporal cortex tissues from PSEN1 mutation carriers (n=11) and from APP mutation carriers (n=7) were measured using an amylin ELISA. Amylin-Aβ structural characteristics of a neuritic plaque in the temporal cortex tissue from a patient with sAD. Brain tissue was stained with anti-amylin antibody (green) and anti-Aβ antibody (red) and analyzed using confocal microscopy (overlay). Scale bar, 10 μm.

FIG. 5F shows Amylin levels in homogenates of human temporal cortex tissues from PSEN1 mutation carriers (n=11) and from APP mutation carriers (n=7) were measured using an amylin ELISA. The relationship between CSF amylin levels and age in same individuals as in FIG. 1k-m; AD groups. Correlation analysis.

FIG. 5G shows Amylin levels in homogenates of human temporal cortex tissues from PSEN1 mutation carriers (n=11) and from APP mutation carriers (n=7) were measured using an amylin ELISA. The relationship between CSF amylin levels and age in same individuals as in FIG. 1k-m; MCI groups.

FIG. 5H shows Amylin levels in homogenates of human temporal cortex tissues from PSEN1 mutation carriers (n=11) and from APP mutation carriers (n=7) were measured using an amylin ELISA. The relationship between CSF amylin levels and age in same individuals as in FIG. 1k-m; CN groups. correlation analysis.

FIG. 5I shows Amylin levels in homogenates of human temporal cortex tissues from PSEN1 mutation carriers (n=11) and from APP mutation carriers (n=7) were measured using an amylin ELISA. Confocal microscopy analysis of brain tissue from a patient with fAD (PSEN1 S132A) triple stained with Thioflavin S (ThioS, green), anti-amylin antibody (red) and anti-Aβ antibody (magenta) showing a presumable capillary stained with ThioS and amylin but negative for Aβ. Scale bar, 10 μm.

FIG. 5J shows Typical characteristics of amylin-associated pathology in the white matter regions of fAD brains. Immunohistochemistry with anti-amylin antibody (red) and anti-Aβ antibody (blue) on a brain section from a fAD patient (PSEN1 I202F) showing amylin immunoreactivity signal in old infarct areas (arrows), perivascular region (arrow heads) and diffusive plaques (double arrow heads). Scale bar, 200 μm.

FIG. 6A shows Vascular phenotypes tested in ADHIP vs AD rats (n=10 rats/group) at 12 M and 16 M. Spontaneous use of forelimbs assessed from forepaws-to-wall contact time during rearing up in a plastic clear cylinder.

FIG. 6B shows Vascular phenotypes tested in ADHIP vs AD rats (n=10 rats/group) at 12 M and 16 M. Balance ability assessed from the angle at falling on the inclined plane.

FIG. 6C shows Vascular phenotypes tested in ADHIP vs AD rats (n=10 rats/group) at 12 M and 16 M. Vestibulomotor function measured by Rotarod.

FIG. 6D shows Vascular phenotypes tested in ADHIP vs AD rats (n=10 rats/group) at 12 M and 16 M. Gait abnormality assessed from hind limb clasping score.

FIG. 6E shows Vascular phenotypes tested in ADHIP vs AD rats (n =10 rats/group) at 12 M and 16 M. Phenotypes associated with cognitive impairment. Short-term recognition memory assessed from novel object recognition test.

FIG. 6F shows Vascular phenotypes tested in ADHIP vs AD rats (n=10 rats/group) at 12 M and 16 M. Phenotypes associated with cognitive impairment. Short-term recognition memory assessed Percentage of time spent in the target quadrant in Morris water maze test.

FIG. 6G shows Vascular phenotypes tested in ADHIP vs AD rats (n=10 rats/group) at 12 M and 16 M. Longitudinal measurement of the body weights of AD and AD-AKO (n=6 rats/group; littermates from different litters).

FIG. 6H shows Vascular phenotypes tested in ADHIP vs AD rats (n=10 rats/group) at 12 M and 16 M. Behavior testing of AD-AKO rats vs AD rats at 12 M and 16 M of age (n =6 rats/group; same rats as in g), including hind limb clasping

FIG. 61 shows Vascular phenotypes tested in ADHIP vs AD rats (n=10 rats/group) at 12 M and 16 M. Behavior testing of AD-AKO rats vs AD rats at 12 M and 16 M of age (n=6 rats/group; same rats as in g), including spontaneous forelimb use

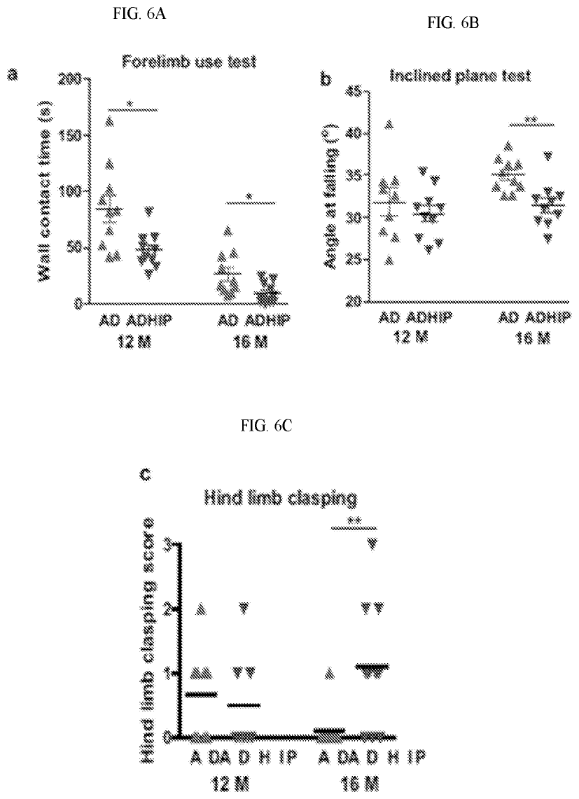

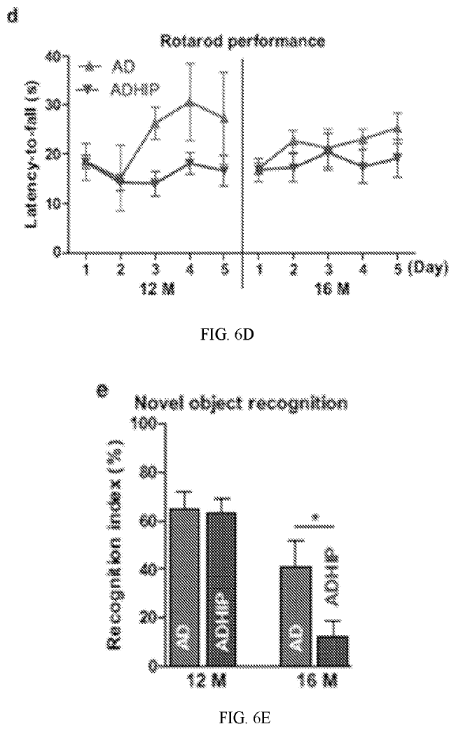

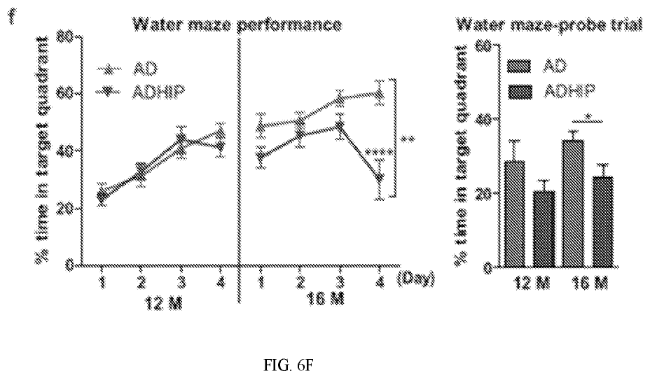

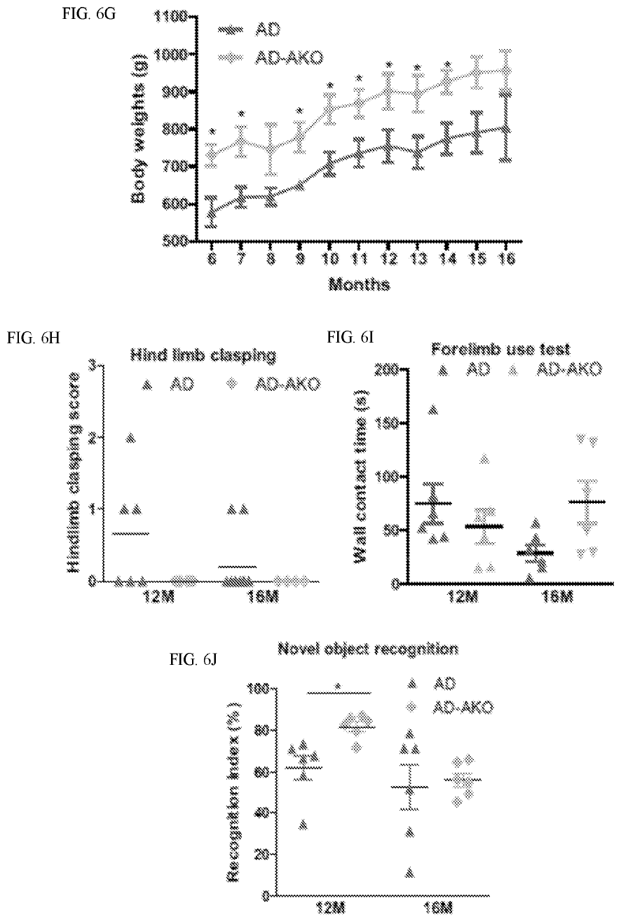

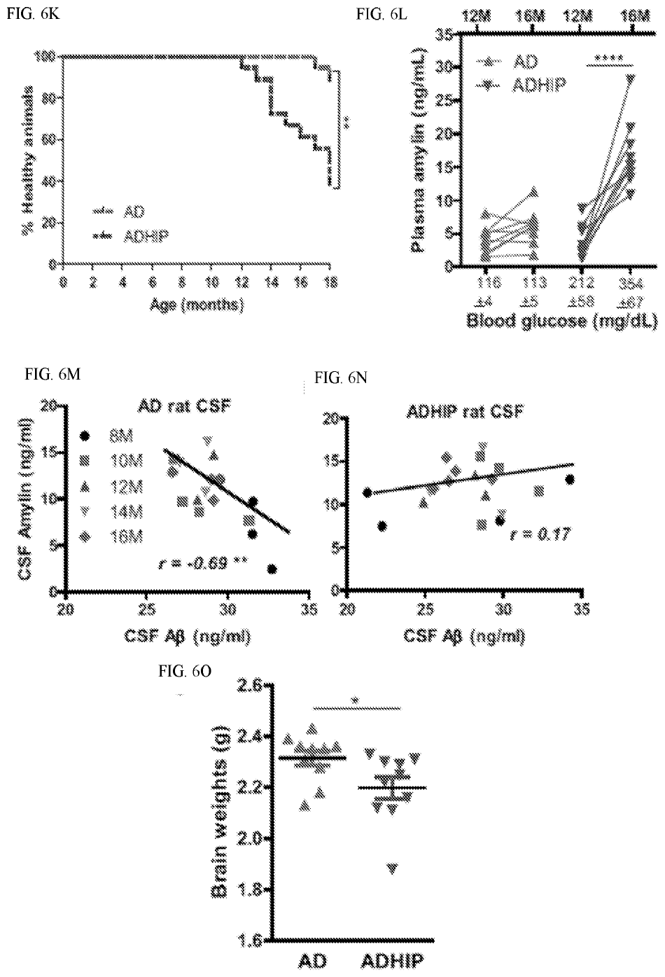

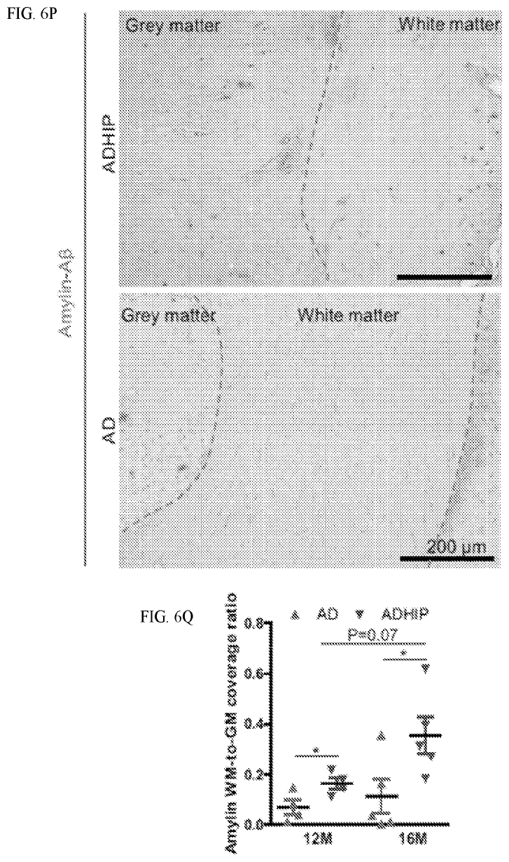

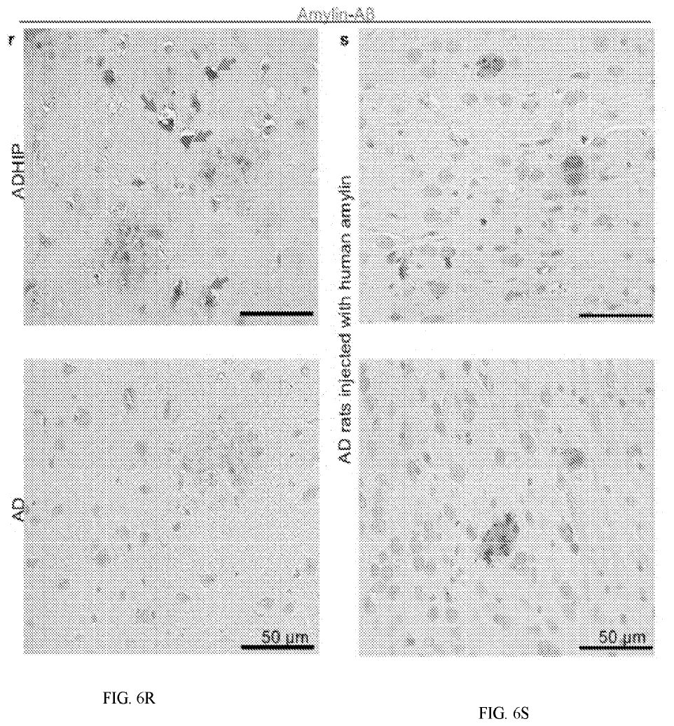

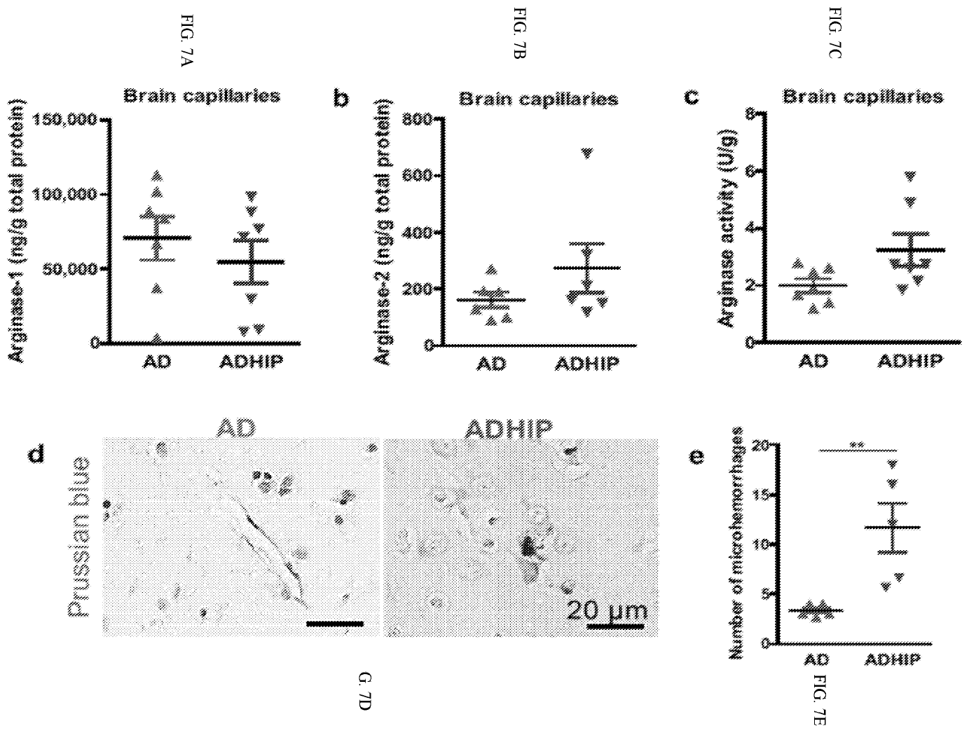

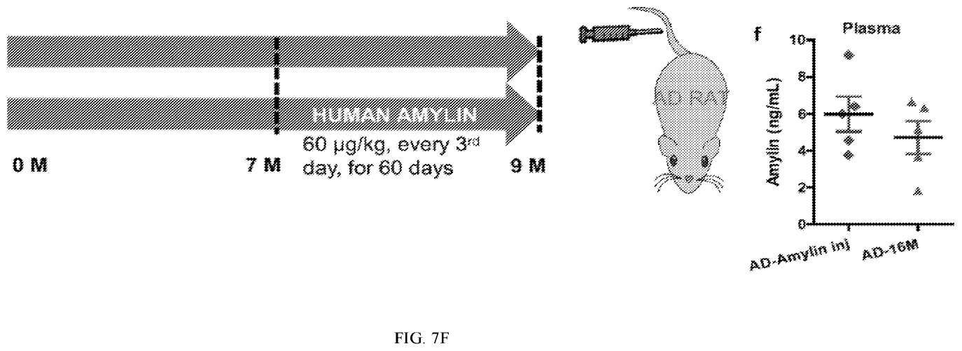

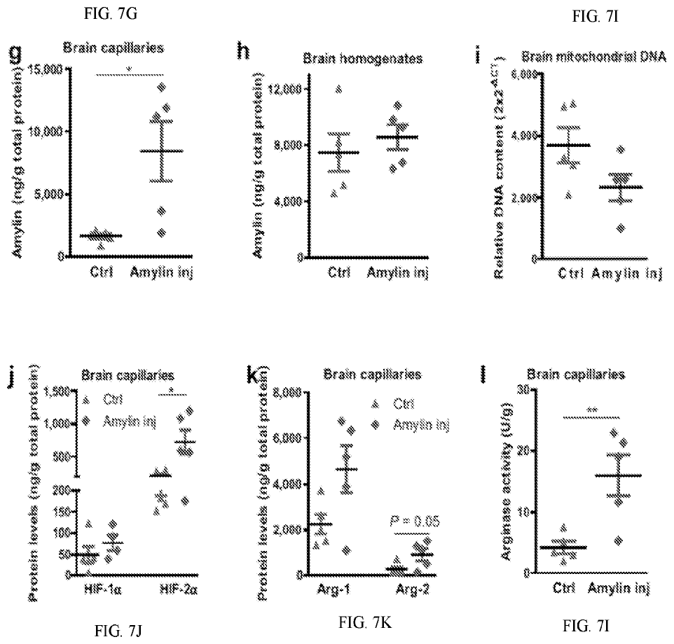

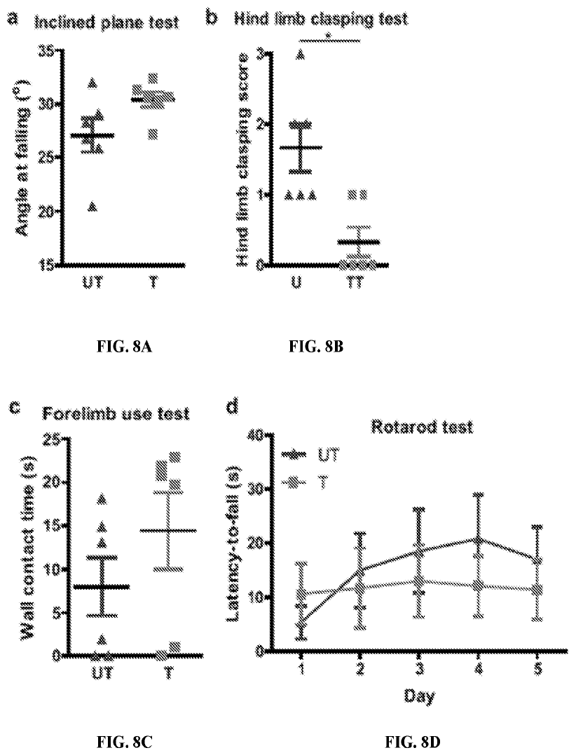

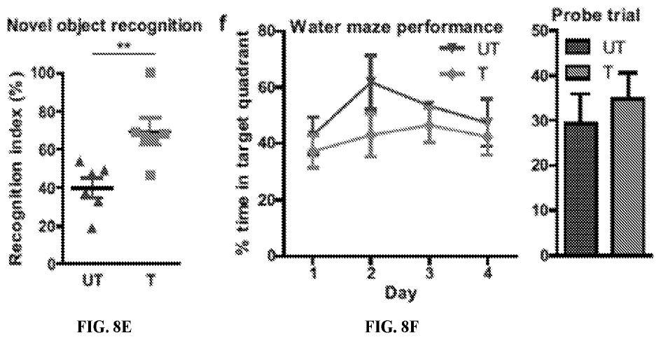

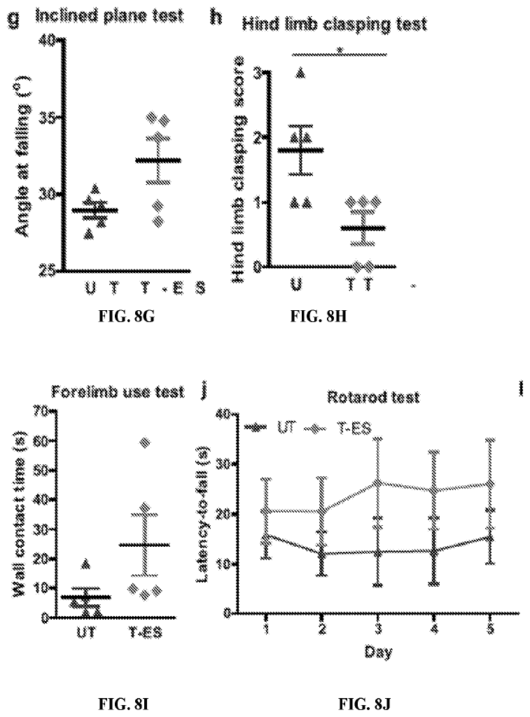

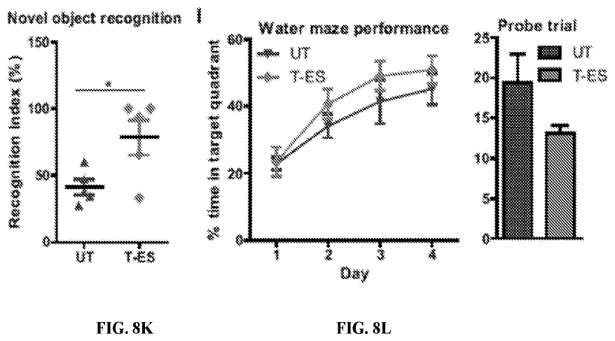

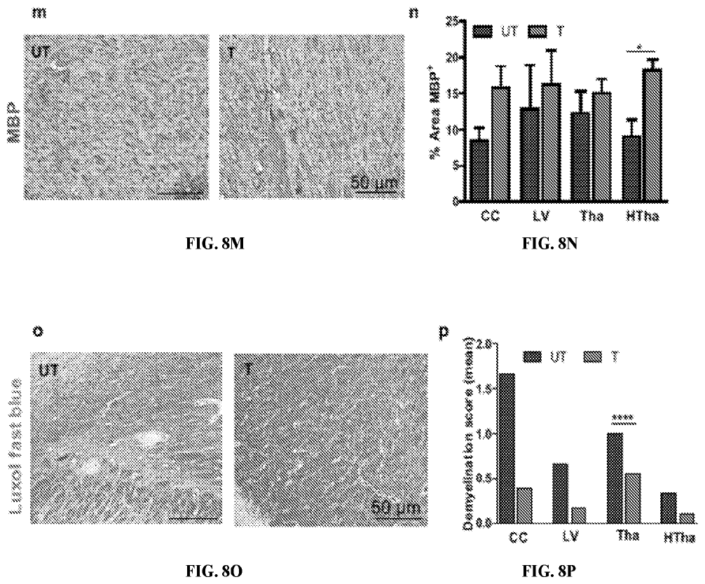

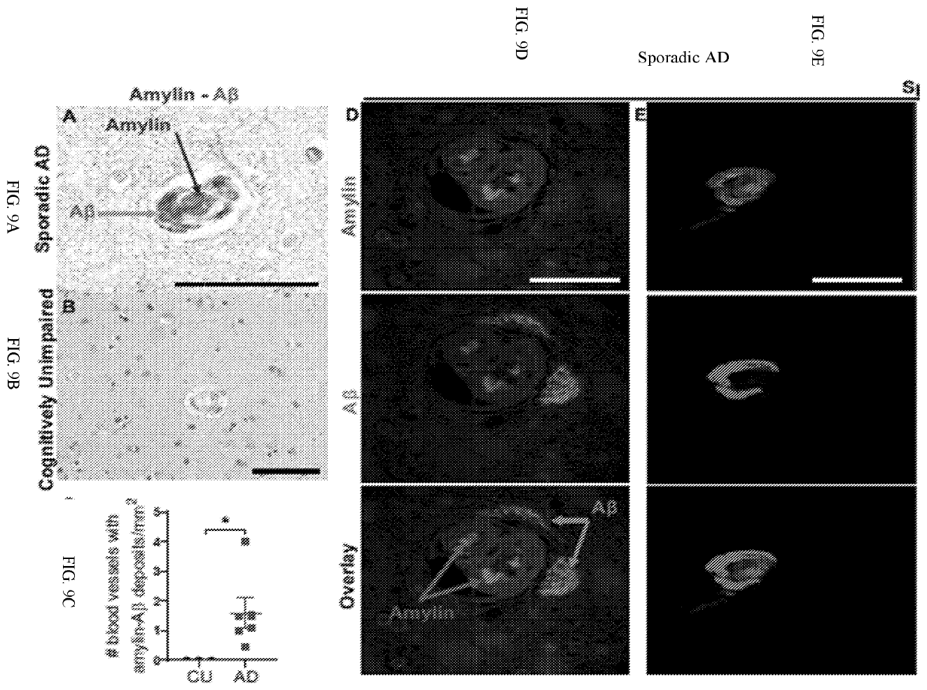

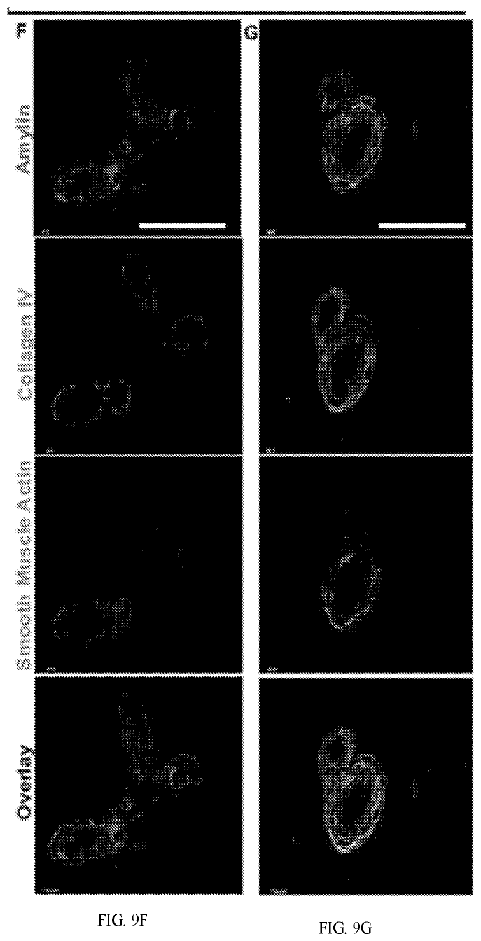

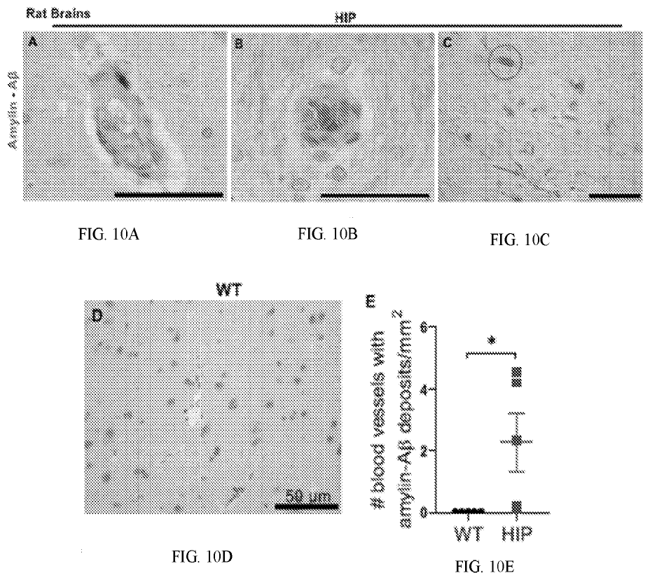

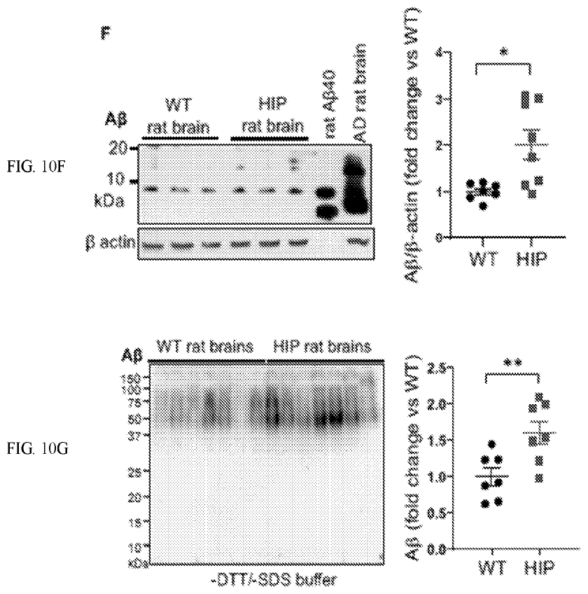

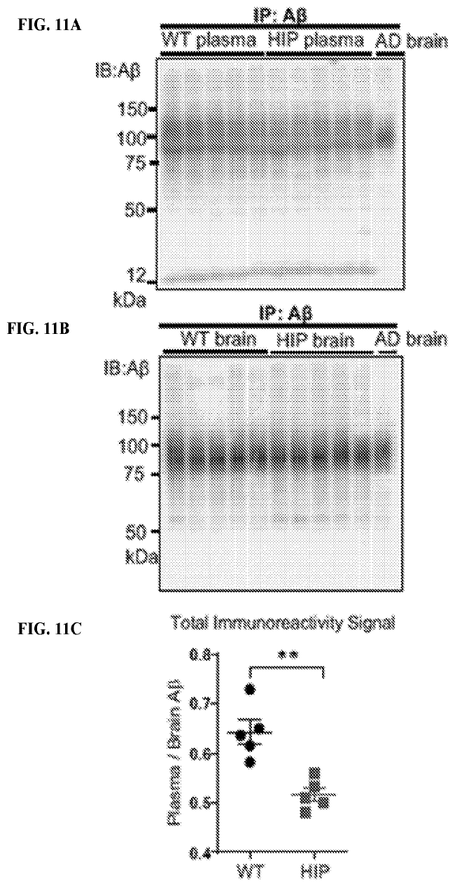

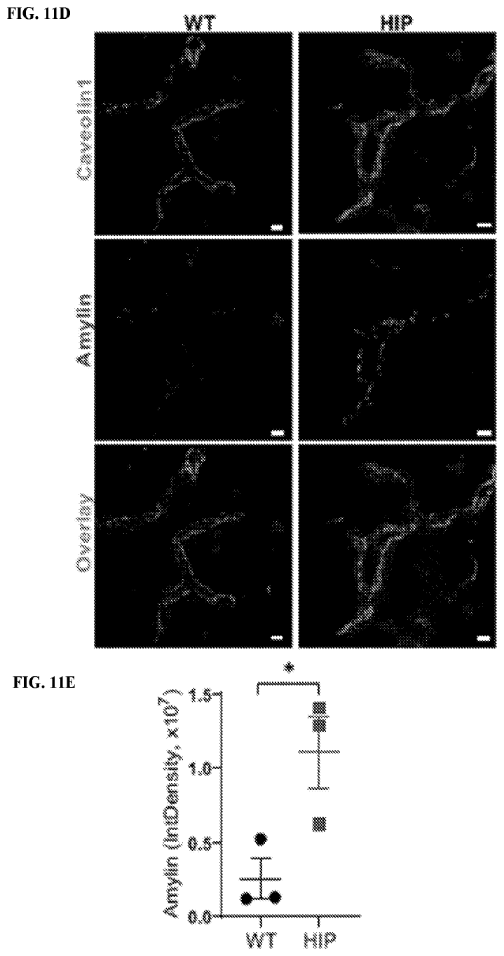

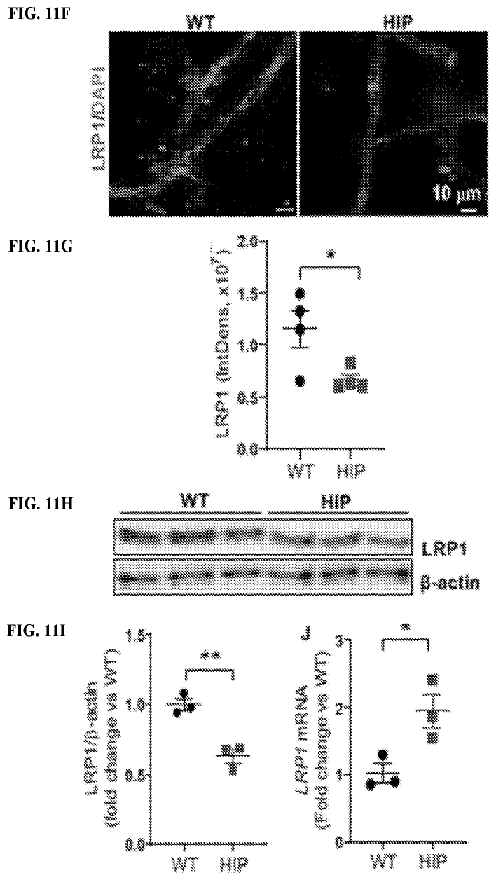

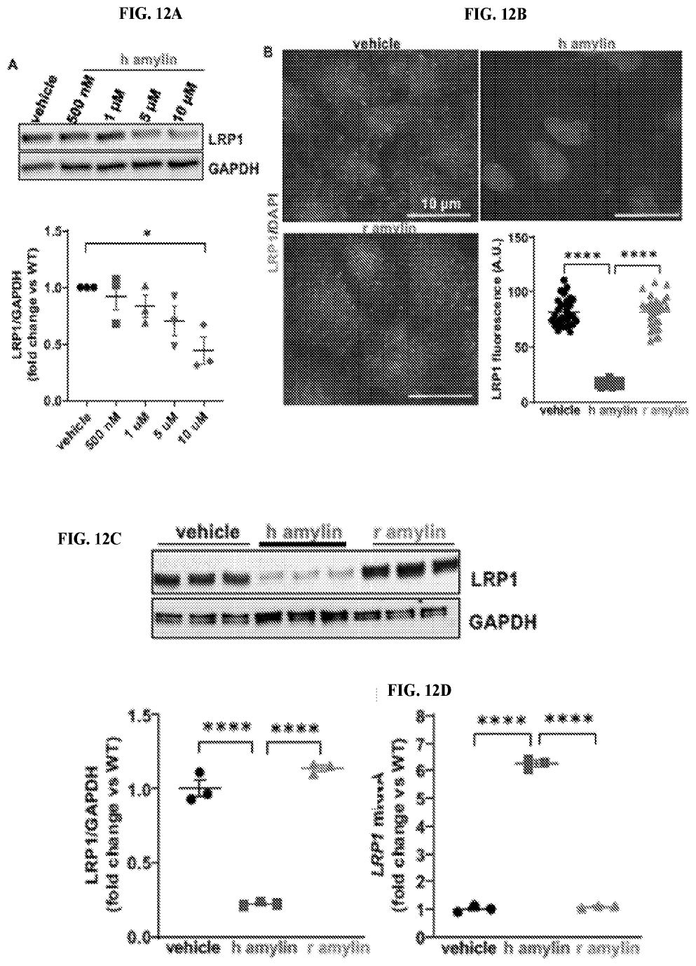

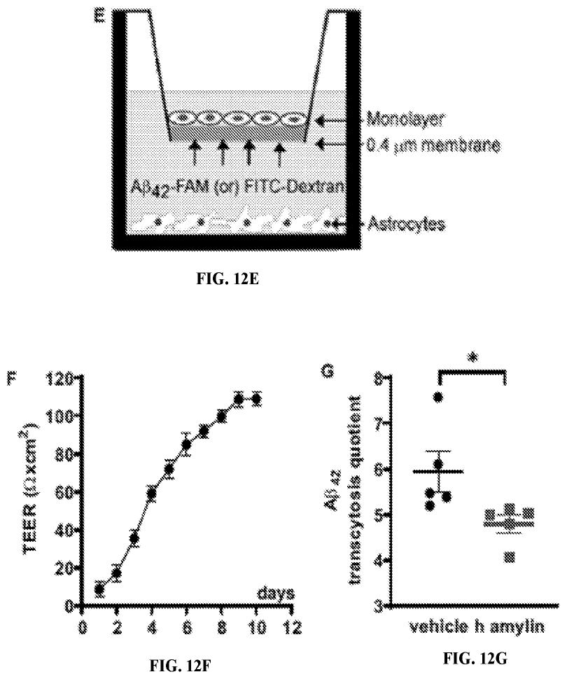

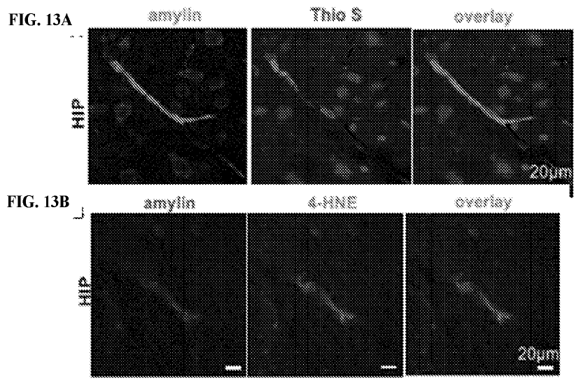

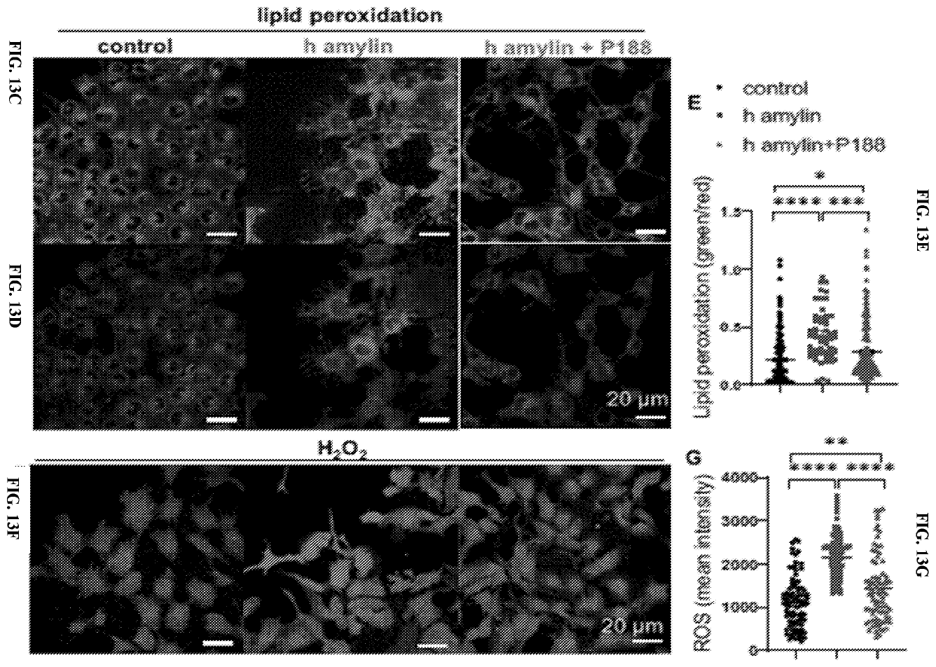

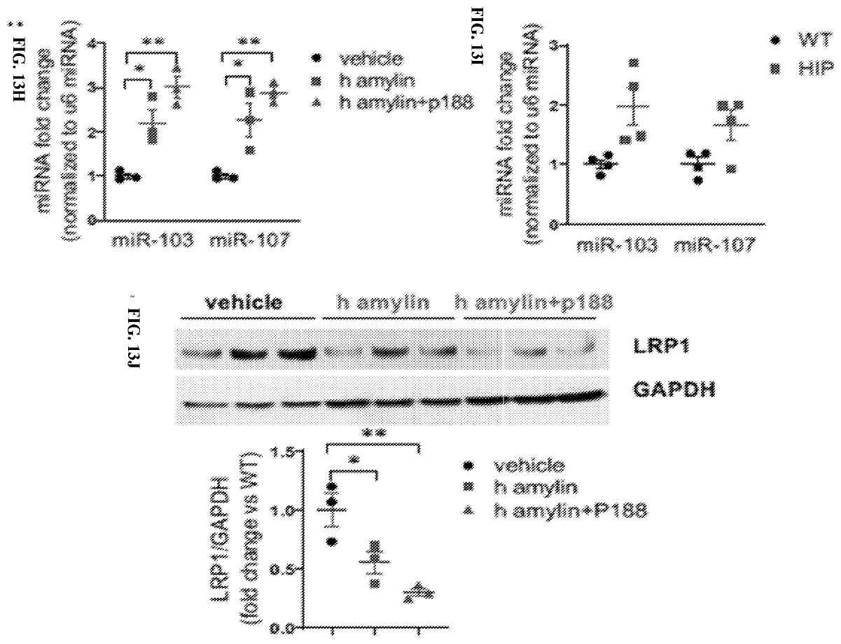

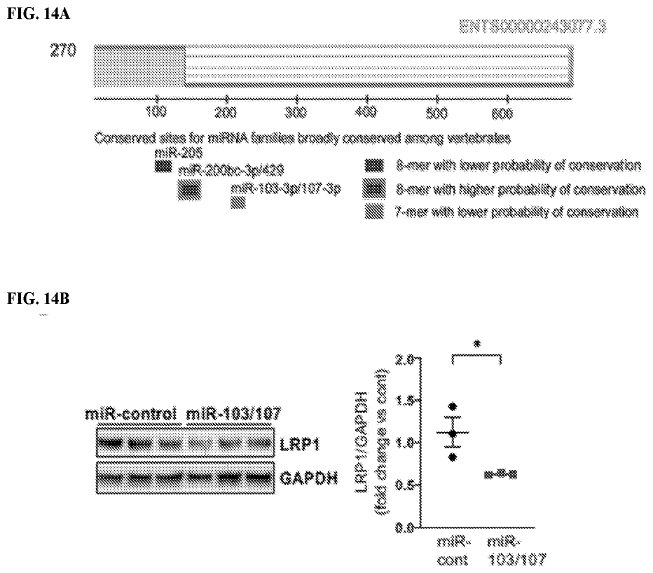

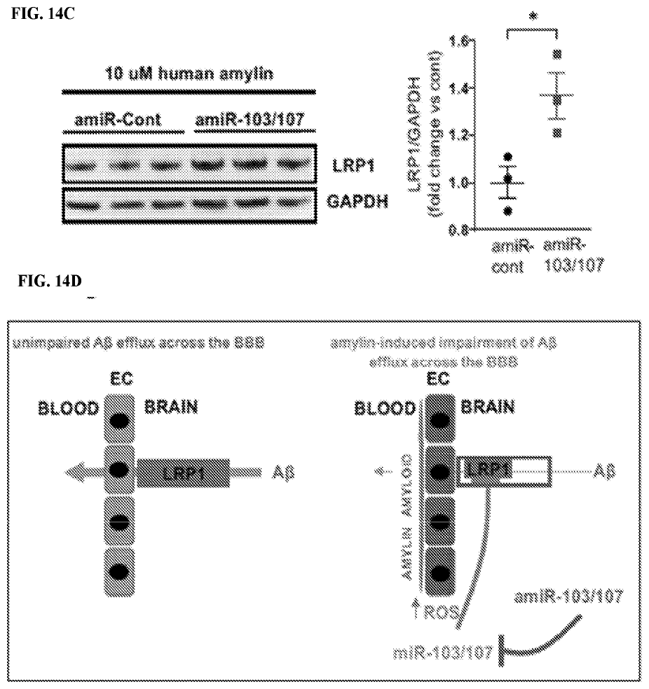

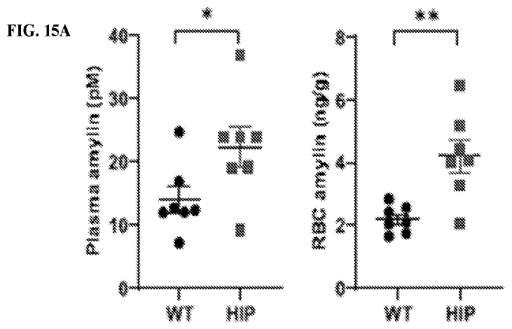

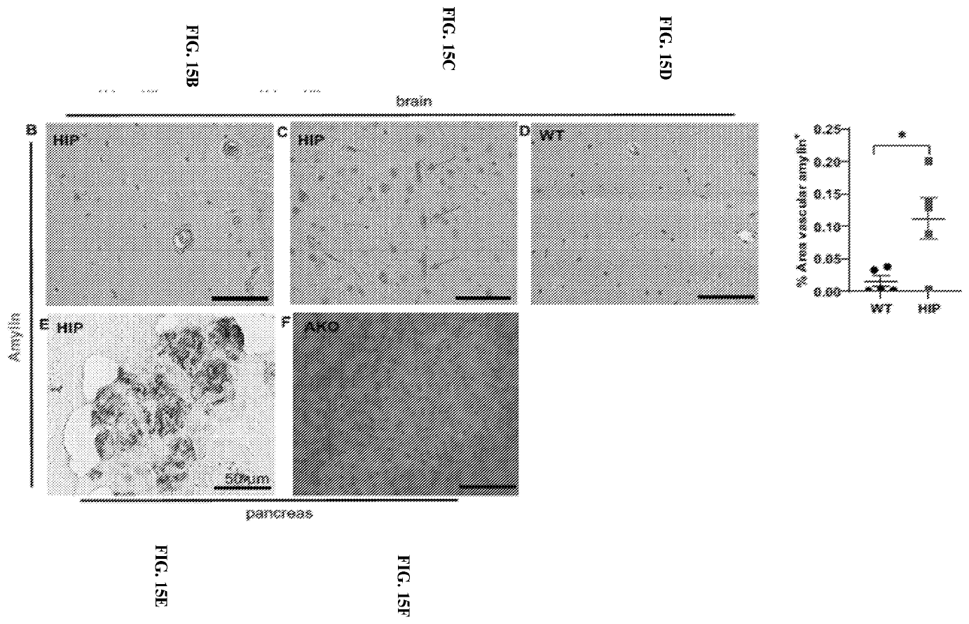

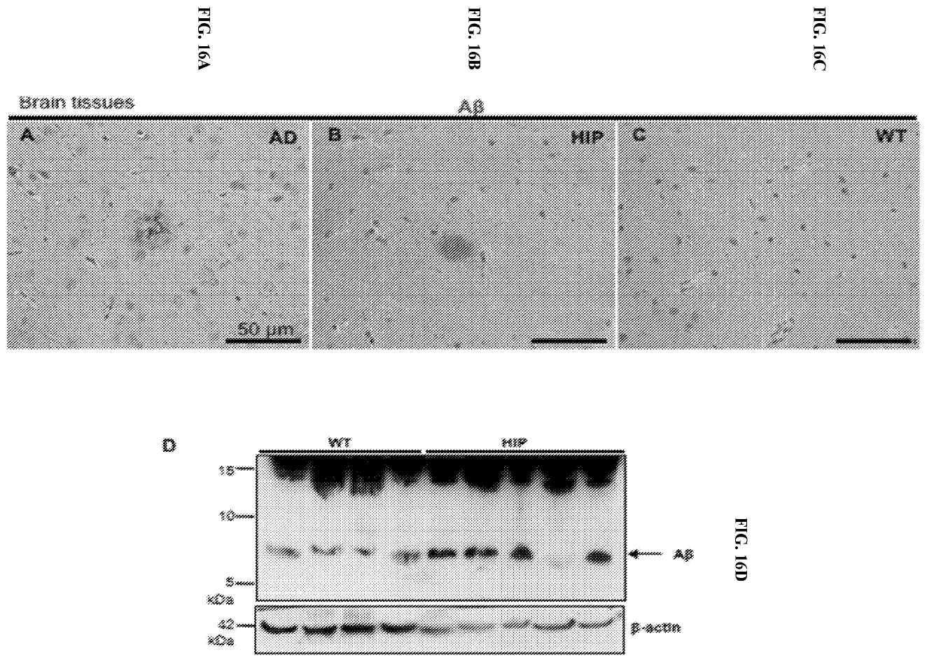

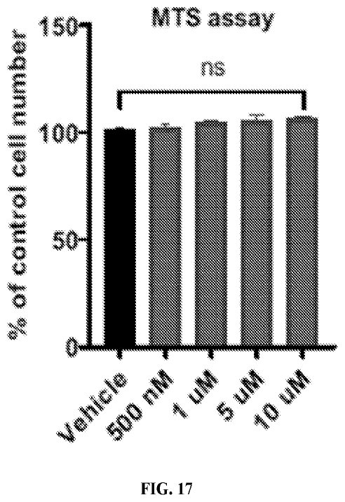

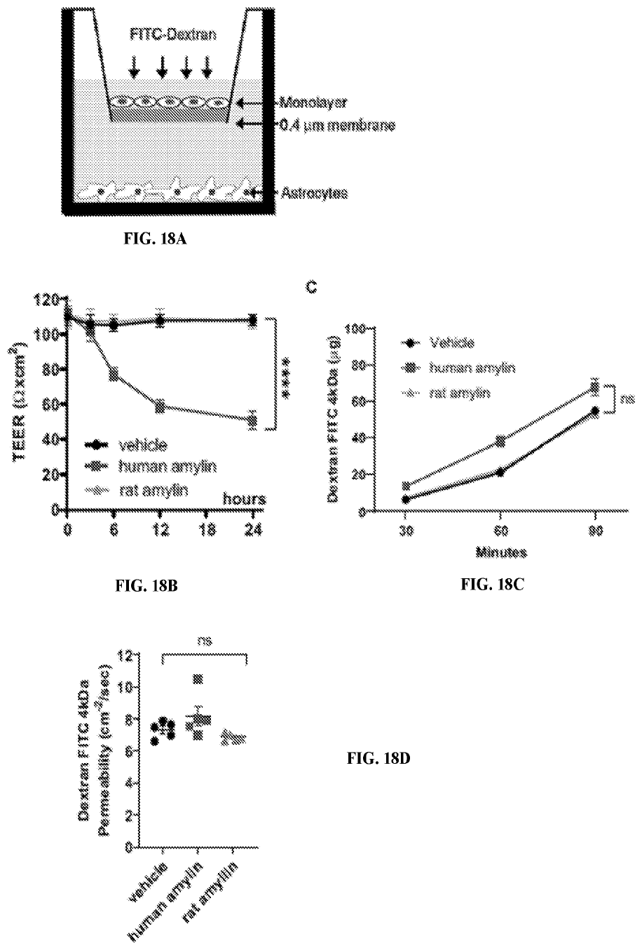

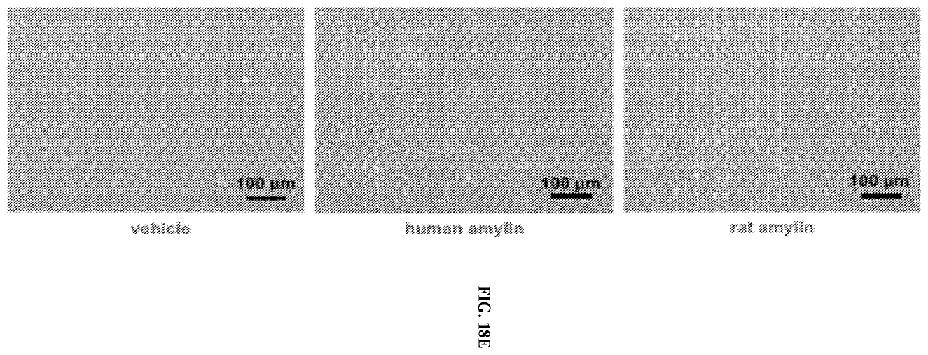

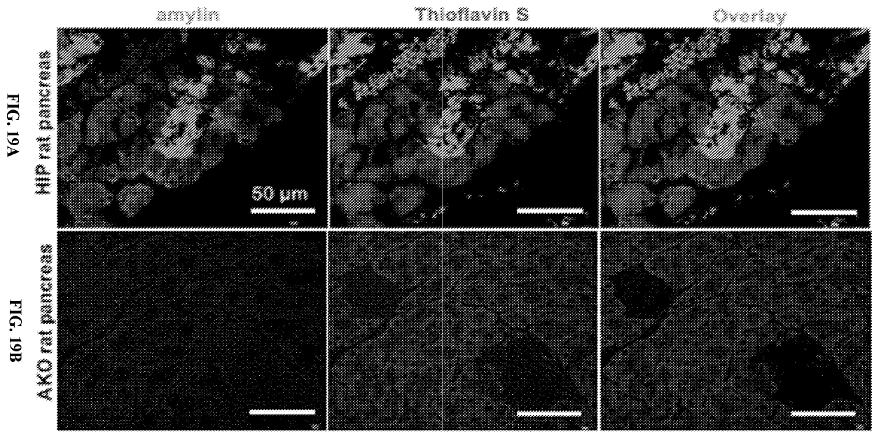

FIG. 6J shows Vascular phenotypes tested in ADHIP vs AD rats (n=10 rats/group) at 12 M and 16 M. Behavior testing of AD-AKO rats vs AD rats at 12 M and 16 M of age (n=6 rats/group; same rats as in g), including novel object recognition tasks

FIG. 6K shows Vascular phenotypes tested in ADHIP vs AD rats (n=10 rats/group) at 12 M and 16 M. Percentage of ADHIP and AD rats (n=17 rats/group; littermates from different litters) with normal functionality, i.e., without glucose dysregulation, lethargy, abnormal gait, or sarcopenia, assessed in a 18 months longitudinal study. Log-rank test.

FIG. 6L shows Vascular phenotypes tested in ADHIP vs AD rats (n=10 rats/group) at 12 M and 16 M. Plasma amylin and blood glucose levels in ADHIP and AD rats (n=10 rats/group; littermates from different litters) at 12 M and 16 M of age.

FIG. 6M shows Vascular phenotypes tested in ADHIP vs AD rats (n=10 rats/group) at 12 M and 16 M. Correlation analyses of CSF amylin vs CSF total Aβ levels in AD rats at 8, 10, 12, 14, and 16M (n=3-5 rats/group).

FIG. 6N shows Vascular phenotypes tested in ADHIP vs AD rats (n=10 rats/group) at 12 M and 16 M. Correlation analyses of CSF amylin vs CSF total Aβ levels in ADHIP rats at 8, 10, 12, 14, and 16M (n=3-5 rats/group).

FIG. 60 shows Vascular phenotypes tested in ADHIP vs AD rats (n=10 rats/group) at 12 M and 16 M. Gross brain weights of ADHIP and AD rats (n=10 rats/group; littermates from different litters)

FIG. 6P shows Vascular phenotypes tested in ADHIP vs AD rats (n=10 rats/group) at 12 M and 16 M. Representative images of immunohistochemical analysis with anti-amylin antibody (brown) and anti-Aβ antibody (green) in white matter (WM) and grey matter (GM) regions in ADHIP and AD rats at 16M (n=5 rats/group). Scale bar, 200 μm.

FIG. 6Q shows Vascular phenotypes tested in ADHIP vs AD rats (n=10 rats/group) at 12 M and 16 M. Representative images of immunohistochemical analysis with anti-amylin antibody (brown) and anti-Aβ antibody (green) in white matter (WM) and grey matter (GM) regions in ADHIP and AD rats at 16M (n=5 rats/group). Scale bar, 200 μm.

FIG. 6R shows Vascular phenotypes tested in ADHIP vs AD rats (n=10 rats/group) at 12 M and 16 M. Representative images of immunohistochemical analysis of amylin (brown) and Aβ (green) showing amylin-positive inclusions in neurons (blue arrows) in ADHIP vs AD rats (16 M old rats)

FIG. 6S shows Vascular phenotypes tested in ADHIP vs AD rats (n=10 rats/group) at 12 M and 16 M. Representative images of immunohistochemical analysis of amylin (brown) and Aβ (green) showing amylin-positive inclusions in neurons (blue arrows) in ADHIP vs AD rats (16 M old rats)

FIG. 7A shows The protein levels of Arginase-1 (Arg-1) in brain capillary lysates from 16 M old ADHIP and AD rats (n=7 rats/group). Arg-1, Arg-2 and arginase activity levels were normalized to the total protein input.

FIG. 7B shows The protein levels of Arginase-2 (Arg-2) in brain capillary lysates from 16 M old ADHIP and AD rats (n=7 rats/group). Arg-1, Arg-2 and arginase activity levels were normalized to the total protein input.

FIG. 7C shows The protein levels of arginase activity in brain capillary lysates from 16 M old ADHIP and AD rats (n=7 rats/group). Arg-1, Arg-2 and arginase activity levels were normalized to the total protein input.

FIG. 7D shows The protein levels of Histological analysis of microhemorrhages in ADHIP vs AD rats stained with Prussian blue dye (n=5 rats/group). Scale bar 20 μm.

FIG. 7E shows The protein levels of Histological analysis of microhemorrhages in ADHIP vs AD rats stained with Prussian blue dye (n=5 rats/group). Scale bar 20 μm.

FIG. 7F shows The results of chronic intravenous infusion of human amylin in AD rats (60 μg/kg of human amylin, every 3 days for 60 days). The levels of amylin in the plasma from amylin-injected AD rats vs AD rats (n=5 rats/group) measured by ELISA.

FIG. 7G The results of chronic intravenous infusion of human amylin in AD rats (60 μg/kg of human amylin, every 3 days for 60 days). The levels of amylin in the plasma from amylin-injected AD rats vs AD rats (n=5 rats/group) measured by ELISA. Amylin levels in the brain capillaries of amylin-injected AD rats vs AD control rats (n=5 rats/group) were measured using an amylin ELISA.

FIG. 7H The results of chronic intravenous infusion of human amylin in AD rats (60 μg/kg of human amylin, every 3 days for 60 days). Amylin levels in brain homogenates (the Guanidine HCl-soluble fraction) of amylin-injected AD rats vs AD control rats (n=5 rats/group) were measured using an amylin ELISA. Levels of amylin were normalized to the total protein input.

FIG. 7I shows Relative mitochondrial DNA content from brains of amylin-injected AD rats vs AD control rats (n=5 rats/group).

FIG. 7J shows. The protein levels of HIF-1α and HIF-2α; levels of arginase activity from amylin-injected AD rats vs AD control rats (n=5 rats/group). HIF-1α, HIF-2α, Arg-1, Arg-2 and arginase activity levels were normalized to the total protein input. Data are means±SEM; P<0.05 *, P<0.01 **; by two-tailed, unpaired Student's t test.

FIG. 7K The protein levels of Arg-1 and Arg-2; in brain capillary lysates from amylin-injected AD rats vs AD control rats (n=5 rats/group). HIF-1α, HIF-2α, Arg-1, Arg-2 and arginase activity levels were normalized to the total protein input. Data are means±SEM; P<0.05 *, P<0.01 **; by two-tailed, unpaired Student's t test.

FIG. 7L shows The protein levels of levels of arginase activity in brain capillary lysates from amylin-injected AD rats vs AD control rats (n=5 rats/group). HIF-1α, HIF-2α, Arg-1, Arg-2 and arginase activity levels were normalized to the total protein input. Data are means±SEM; P<0.05 *, P<0.01 **; by two-tailed, unpaired Student's t test.

FIG. 8A shows End-point (18 M) behavior testing of T vs UT rats (n=6 rats/group), including inclined plane test, Data are means±SEM (by two-tailed, unpaired Student's t test).

FIG. 8B shows End-point (18 M) behavior testing of T vs UT rats (n=6 rats/group), including pathologic hind limb clasping Data are means±SEM; P<0.05 *, P<0.01 **, P<0.0001 ****; by two-tailed, unpaired Student's t test.

FIG. 8C shows End-point (18 M) behavior testing of T vs UT rats (n=6 rats/group), including inclined spontaneous forelimb use. Data are means±SEM, means; P<0.05 *, P<0.01 **, P<0.0001 ****; by two-tailed, unpaired Student's t test.

FIG. 8D shows End-point (18 M) behavior testing of T vs UT rats (n=6 rats/group), including inclined Rotarod test, Data are means±SEM; P<0.05 *, P<0.01 **, P<0.0001 ****; by two-tailed, unpaired Student's t test, repeated measures ANOVA with Bonferroni post-hoc.

FIG. 8E shows End-point (18 M) behavior testing of T vs UT rats (n=6 rats/group), including novel object recognition test. Data are means±SEM; P<0.05 *, P<0.01 **, P<0.0001 ****; by two-tailed, unpaired Student's t test.

FIG. 8F shows End-point (18 M) behavior testing of T vs UT rats (n=6 rats/group), including Morris water maze Data are means±SEM; P<0.05 *, P<0.01 **, P<0.0001 ****; by two-tailed, unpaired Student's t test, repeated measures ANOVA with Bonferroni post-hoc.

FIG. 8G shows End-point (16 M) behavior testing of T-ES vs UT rats (n=5 rats/group), including inclined plane test Data are means±SEM; P<0.05 *, P<0.01 **, P<0.0001 ****; by two-tailed, unpaired Student's t test.

FIG. 8H shows End-point (16 M) behavior testing of T-ES vs UT rats (n=5 rats/group), including pathologic hind limb clasping Data are means±SEM; P<0.05 *, P<0.01 **, P<0.0001 ****; by two-tailed, unpaired Student's t test.

FIG. 8I shows End-point (16 M) behavior testing of T-ES vs UT rats (n=5 rats/group), including spontaneous forelimb use Data are means±SEM, P<0.05 *, P<0.01 **, P<0.0001 ****; by two-tailed, unpaired Student's t test.

FIG. 8J shows End-point (16 M) behavior testing of T-ES vs UT rats (n=5 rats/group), including Rotarod test Data are means±SEM, P<0.05 *, P<0.01 **, P<0.0001 ****; by repeated measures ANOVA with Bonferroni post-hoc.

FIG. 8K shows End-point (16 M) behavior testing of T-ES vs UT rats (n=5 rats/group), including inclined novel object recognition test. Scale bar, 50 μm (CC—corpus callosum; LV—lateral ventricle area; Tha—thalamus; HTha—hypothalamus). Data are means±SEM; P<0.05 *, P<0.01 **, P<0.0001 ****; by two-tailed, unpaired Student's t test.

FIG. 8L shows End-point (16 M) behavior testing of T-ES vs UT rats (n=5 rats/group), including Morris water maze Data are means±SEM; P<0.05 *, P<0.01 **, P<0.0001 ****; by two-tailed, unpaired Student's t test, repeated measures ANOVA with Bonferroni post-hoc.

FIG. 8M shows Representative images and immunohistochemical analyses of MBP in brains of T vs UT rats (n=6 rats/group). Representative images are the brain hypothalamus. Scale bar, 50 μm (CC—corpus callosum; LV—lateral ventricle area; Tha—thalamus; HTha—hypothalamus). Data; P<0.05 *, P<0.01 **, P<0.0001 ****.

FIG. 8N shows Representative images and immunohistochemical analyses of MBP in brains of T vs UT rats (n=6 rats/group). Representative images are the brain hypothalamus. Scale bar, 50 μm (CC—corpus callosum; LV—lateral ventricle area; Tha—thalamus; HTha—hypothalamus). Data are means±SEM.

FIG. 8O shows Representative images and immunohistochemical analyses of LFB staining in brains of T vs UT rats (n=6 rats/group). Representative images are the brain hypothalamus. Scale bar, 50 μm (CC—corpus callosum; LV—lateral ventricle area; Tha—thalamus; HTha—hypothalamus).

FIG. 8P shows Representative images and immunohistochemical analyses of LFB staining in brains of T vs UT rats (n=6 rats/group). Representative images are the brain hypothalamus. Scale bar, 50 μm (CC—corpus callosum; LV—lateral ventricle area; Tha—thalamus; HTha—hypothalamus). Data are means; P<0.05 *, P<0.01 **, P<0.0001 ****; by Mann-Whitney test.

FIG. 9 shows: Amylin-Aβ interaction at the blood-brain interface in human AD brains. Representative immunohistochemical (IHC) micrographs of brain sections from patients with sporadic AD

FIG. 9B shows Amylin-Aβ interaction at the blood-brain interface in human AD brains. Representative immunohistochemical (IHC) micrographs of brain sections from patients cognitively unimpaired (CU) individuals that were co-stained with anti-amylin (brown) and anti-Aβ (green) antibodies.

FIG. 9C shows Representative immunohistochemical (IHC) micrographs of brain sections from patients with sporadic AD. Analysis of the number of blood vessels with vascular mixed amylin-Aβ deposition (per mm²) in brains from AD (n=6) and CU (n=3) (2 slides/brain) groups assessed from IHC. Data are mean±SEM. P≤0.05 *; by two-tailed, unpaired t test.

FIG. 9D shows Representative images of confocal microscopy analysis of brain sections from the same AD individuals (n=6; 2 slides/brain) as in above showing amylin (green) and Aβ (red) deposits in small-type vessels in which amylin is present on the luminal side.

FIG. 9E shows Representative images of confocal microscopy analysis of brain sections from the same AD individuals (n=6; 2 slides/brain) as in above showing amylin (green) and Aβ (red) deposits in small-type vessels in which amylin is present within the vessel wall.

FIG. 9F shows Representative immunofluorescence images of AD brain sections triple stained with anti-amylin (green), anti-collagen IV (red) and anti-smooth muscle actin (blue) (n=3; 2 slides/brain). Scale bars, 50 μm.

FIG. 9G shows Representative immunofluorescence images of AD brain sections triple stained with anti-amylin (green), anti-collagen IV (red) and anti-smooth muscle actin (blue) (n=3; 2 slides/brain). Scale bars, 50 μm.

FIG. 10A shows: Pancreatic amylin accumulates in the brain microvasculature and impairs Aβ efflux from the brain. Representative IHC micrographs of brain sections from HIP rats co-stained with anti-amylin (brown) and anti-Aβ (green) antibodies. Scale bars, 50 μm

FIG. 10B shows Pancreatic amylin accumulates in the brain microvasculature and impairs Aβ efflux from the brain. Scale bars, 50 μm Representative IHC micrographs of brain sections from HIP rats co-stained with anti-amylin (brown) and anti-Aβ (green) antibodies.

FIG. 10C shows Pancreatic amylin accumulates in the brain microvasculature and impairs Aβ efflux from the brain. Scale bars, 50 μm Representative IHC micrographs of brain sections from HIP rats co-stained with anti-amylin (brown) and anti-Aβ (green) antibodies.

FIG. 10D shows Pancreatic amylin accumulates in the brain microvasculature and impairs Aβ efflux from the brain. Scale bars, 50 μm Representative IHC micrographs of brain sections from WT rats co-stained with anti-amylin (brown) and anti-Aβ (green) antibodies.

FIG. 10E shows Pancreatic amylin accumulates in the brain microvasculature and impairs Aβ efflux from the brain. E, Analysis of the number of blood vessels with vascular amylin-Aβ deposition (per mm²) in brains of WT and HIP rats (n=5/group) (3 slides/brain), assessed from IHC.Data are mean±SEM. P≤0.05 *, P≤0.01 **; by two-tailed, unpaired t test

FIG. 10F shows Pancreatic amylin accumulates in the brain microvasculature and impairs Aβ efflux from the brain. Representative Western blot and densitometry quantification of Aβ in brain homogenates from HIP rats and WT littermates using acidic urea gel (n=3/group) to resolve monomers. Rat Aβ₄₀ peptide and APP/PS1 rat brain homogenate were used as positive controls. Aβ densitometry was normalized to loading control actin. Aβ densitometry quantification was calculated from two experiments (n=7-8/group). Data are mean±SEM. P≤0.05 *, P≤0.01 **; by two-tailed, unpaired t test

FIG. 10G shows Pancreatic amylin accumulates in the brain microvasculature and impairs Aβ efflux from the brain. Representative Western blot and densitometry analyses of aggregated Aβ in brain homogenates from HIP rats and WT littermates (n=7/group) using native-PAGE. Data are mean±SEM. P≤0.05 *, P≤0.01 **; by two-tailed, unpaired t test

FIG. 11A shows: High blood amylin levels downregulate the Aβ efflux transporter at the BBB. Immunoprecipitation and western blot analyses of Aβ in the plasma from WT and HIP rats (n=5/group). Age-matched APP/PS1 rat brain homogenate were used as a positive control.

FIG. 11B shows: High blood amylin levels downregulate the Aβ efflux transporter at the BBB. Immunoprecipitation and western blot analyses of Aβ in brain homogenates from WT and HIP rats (n=5/group). Age-matched APP/PS1 rat brain homogenate were used as a positive control.

FIG. 11C shows: High blood amylin levels downregulate the Aβ efflux transporter at the BBB. Ratio of plasma Aβ-to-brain Aβ levels in HIP and WT rats assessed from Western blot analysis of Aβ enriched by immunoprecipitation from plasma and brain homogenates (A, B). Data are mean±SEM. P≤0.05 *, P≤0.01 ** by two-tailed, unpaired t test

FIG. 11D shows: High blood amylin levels downregulate the Aβ efflux transporter at the BBB. Confocal fluorescent micrographs of amylin (green) and endothelial cell marker caveolin-1 (red) in brain capillaries isolated from HIP rats and WT littermates. Scale bars, 10 μm

FIG. 11E shows: High blood amylin levels downregulate the Aβ efflux transporter at the BBB. Levels of amylin fluorescence intensity signals quantified from n=21 and n=47 isolated capillaries from WT rats and HIP littermates (n=3 rats/group), respectively, and averaged for each rat. Data are mean±SEM. P≤0.05 *, P≤0.01 ** by two-tailed, unpaired t test

FIG. 11F shows: High blood amylin levels downregulate the Aβ efflux transporter at the BBB. Confocal immunofluorescent micrographs of LRP1 (green) and nuclei (blue) in isolated capillaries from WT rats and HIP littermates. Scale bars, 10 μm

FIG. 11G shows: High blood amylin levels downregulate the Aβ efflux transporter at the BBB. Levels of LRP1 fluorescence intensity signals quantified from WT rats (n=4) and HIP littermates (n=4) and averaged for each rat. Data are mean±SEM. P≤0.05 *, P≤0.01 ** by two-tailed, unpaired t test

FIG. 11H shows: High blood amylin levels downregulate the Aβ efflux transporter at the BBB. Western blot and densitometry quantification of LRP1 in brain capillary lysates isolated from HIP rats and WT littermates (n=3 rats/group).

FIG. 11I shows: High blood amylin levels downregulate the Aβ efflux transporter at the BBB. Western blot and densitometry quantification of LRP1 in brain capillary lysates isolated from HIP rats and WT littermates (n=3 rats/group). Data are mean±SEM. P≤0.05 *, P≤0.01 ** by two-tailed, unpaired t test

FIG. 11J shows: High blood amylin levels downregulate the Aβ efflux transporter at the BBB. LRP1 mRNA levels (fold difference using 2^{-ΔΔCt} method) in brain capillary lysates isolated from the same HIP and WT rats as in (H) (n=3 rats/group). Data are mean±SEM. P≤0.05 *, P≤0.01 ** by two-tailed, unpaired t test

FIG. 12A shows: In vitro test of amylin-induced impairment of Aβ efflux across the BBB. Representative Western blot and densitometry quantification of LRP1 in lysates from primary rat brain microvascular vascular endothelial cells (ECs) treated with vehicle or various concentrations of human amylin (500 nM, 1 μM, 5 μM, and 10 μM) for 24 hours (n=3 preparations/test). Data are mean±SEM. P≤0.05 *, P≤0.0001 ****; by one-way ANOVA with Dunnett's post-hoc.

FIG. 12B shows In vitro test of amylin-induced impairment of Aβ efflux across the BBB. Immunofluorescent micrographs (LRP1-red; nuclei-blue) and quantification of LRP1 in ECs treated with vehicle, human amylin (10 μM) or rat amylin (10 μM) (n=30 cells/group from 3 replicates per group). Scale bars, 10 μm Data are mean±SEM. P≤0.05 *, P≤0.0001 ****; by one-way ANOVA with Tukey's post-hoc.

FIG. 12C shows In vitro test of amylin-induced impairment of Aβ efflux across the BBB. Western blot and densitometry quantification of LRP1 in lysates from ECs treated with vehicle, human amylin or rat amylin as in (B) (n=3 preparations/test). Data are mean±SEM. P≤0.05 *, P≤0.0001 ****; by one-way ANOVA with Tukey's post-hoc.

FIG. 12D shows In vitro test of amylin-induced impairment of Aβ efflux across the BBB. LRP1 mRNA levels (fold difference using 2^{-ΔΔCt} method) measured with qRT-PCR in lysates from ECs treated with vehicle, human amylin or rat amylin (same 3 preparations/test used in panels B and C). Data are mean±SEM. P≤0.05 *, P≤0.0001 ****; by one-way ANOVA with Tukey's post-hoc.

FIG. 12E shows In vitro test of amylin-induced impairment of Aβ efflux across the BBB. Cartoon representation of the in vitro BBB model (ECs monolayer—luminal chamber; astrocytes—abluminal chamber) used in Aβ transcytosis experiments.

FIG. 12F shows In vitro test of amylin-induced impairment of Aβ efflux across the BBB. Transendothelial electrical resistance (TEER) in EC monolayers (n=20 preparations) as a function of days in culture.

FIG. 12G shows In vitro test of amylin-induced impairment of Aβ efflux across the BBB. The Aβ₄₂ transcytosis quotient (TQ) across the in vitro BBB, in vehicle- and human amylin-treated EC monolayers. two-tailed, unpaired t test.

FIG. 13A shows: MiRNA upregulation and LRP1 downregulation by amylin amyloid-mediated stress in vascular endothelial cells. Representative fluorescent images of Thioflavin S (Thio S, green) and amylin (red) staining in brain tissue sections from HIP rats (n=3 rats) showing the presence of amylin amyloid in a brain capillary. Scale bars, 20 μm

FIG. 13B shows MiRNA upregulation and LRP1 downregulation by amylin amyloid-mediated stress in vascular endothelial cells, Same as in (FIG. 13A) for staining for the lipid peroxidation marker 4-HNE (red) and amylin (green). Scale bars, 20 μm

FIG. 13C shows Representative fluorescent images of lipid peroxidation levels in ECs incubated with human amylin (n=53 cells) or with poloxamer 188 (P188) prior to incubation with human amylin (n=161 cells), compared to controls (n=141 cells). Scale bars, 20 μm

FIG. 13D shows Representative fluorescent images and analysis of lipid peroxidation levels in ECs incubated with human amylin (n=53 cells) or with poloxamer 188 (P188) prior to incubation with human amylin (n=161 cells), compared to controls (n=141 cells). Scale bars, 20 μm

FIG. 13E shows Representative analysis of lipid peroxidation levels in ECs incubated with human amylin (n=53 cells) or with poloxamer 188 (P188) prior to incubation with human amylin (n=161 cells), compared to controls (n=141 cells). Data are mean±SEM. P≤0.05 *, P≤0.01 **, P≤0.001 ***, P≤0.0001 ****; by one-way ANOVA with Tukey's post-hoc

FIG. 13F shows Representative fluorescent images of ROS levels in ECs incubated with human amylin (n=103 cells) or with P188 prior to incubation with human amylin (n=85 cells), compared to controls (n=92 cells). Scale bars, 20 μm

FIG. 13G shows Representative analysis of ROS levels in ECs incubated with human amylin (n=103 cells) or with P188 prior to incubation with human amylin (n=85 cells), compared to controls (n=92 cells). Data are mean±SEM. P≤0.05 *, P≤0.01 **, P≤0.001 ***, P≤0.0001 ****; by one-way ANOVA with Tukey's post-hoc

FIG. 13H shows Expression of miR-103 and miR-107 measured by qRT-PCR (fold difference using 2^{-ΔΔCt} method) in ECs incubated with human amylin or with P188 prior to incubation with human amylin (n=3 preparations/treatment). miR-U6 was used as internal control. Data are mean±SEM. P≤0.05 *, P≤0.01 **, P≤0.001 ***, P≤0.0001 ****; by one-way ANOVA with Tukey's post-hoc.

FIG. 13I shows Expression levels of miRNA (miR)-103 and miR-107 measured by qRT-PCR (fold difference using 2^{-ΔΔCt} method) in lysates from HIP rat brain capillaries compared to those in WT rats (n=4 rats/group). two-tailed, unpaired t test. Data are mean±SEM. P≤0.05 *, P≤0.01 **, P≤0.001 ***, P≤0.0001 ****; by one-way ANOVA with Tukey's post-hoc

FIG. 13J shows Representative Western blot and densitometry quantification of LRP1 from rat brain microvascular ECs treated with human amylin or with poloxamer 188 (P188) prior to incubation with human amylin, compared to control vehicle (n=3 preparations/treatment). Data are mean±SEM. P≤0.05 *, P≤0.01 **, P≤0.001 ***, P ≤0.0001 ****; by one-way ANOVA with Tukey's post-hoc

FIG. 14A shows: Amylin-induced suppression of Aβ transporter is rescued by antisense microRNAs. TargetScan schematic showing consensus regions for miR-205, miR200bc-3p/429, and miR-103 and miR-107. Data are mean±SEM. P≤0.05 *; by two-tailed, unpaired t test.

FIG. 14B shows: Amylin-induced suppression of Aβ transporter is rescued by antisense microRNAs. Western blot and densitometry quantification of LRP1 from miRNA (miR) 103 and miR-107 treated ECs compared to miR-control (n=3 preparations/group). Data are mean±SEM. P≤0.05 *; by two-tailed, unpaired t test.

FIG. 14C shows: Amylin-induced suppression of Aβ transporter is rescued by antisense microRNAs. Western blot and densitometry quantification of LRP1 from antagomir (amiR) 103 and amiR-107 treated ECs compared to amiR-control treated cells (n=3 preparations/group). Data are mean±SEM. P≤0.05 *; by two-tailed, unpaired t test.

FIG. 14D shows: Amylin-induced suppression of Aβ transporter is rescued by antisense microRNAs. Schematic summary of the effect of amyloid-forming human amylin on the Aβ efflux across the BBB and the rescue mechanism. Rats expressing endogenous non-amyloidogenic rat amylin that have unimpaired Aβ efflux across the BBB (left panel). Human amylin-expressing rats have amylin amyloid deposition in the brain microvasculature and impaired Aβ efflux across the BBB (right panel). This was caused by miRNA-based translational repression of LRP1 (red pathway) and was reversed by antisense microRNA (green pathway). Data are mean±SEM. P≤0.05 *.

FIG. 15 shows Immunochemical analyses of amylin in blood, brain and pancreatic tissues from transgenic and wild-type rats. Levels of amylin by ELISA in plasma and lysates of red blood cells (RBCs) from HIP and WT rats (n=7/group). Levels of amylin in the RBC lysates are normalized to the total protein input. Data are means±SEM. P≤0.05 *, P≤0.01 **; by two-tailed, unpaired t test.

FIG. 15B shows Immunochemical analyses of amylin in blood, brain and pancreatic tissues from transgenic and wild-type rats. IHC analysis with anti-amylin antibody (brown) on brain sections from HIP rats (n=5/group) and AKO rat pancreas (negative control for amylin immunoreactivity signal. Scale bars, 50 μm Data are means±SEM. P≤0.05 *, P≤0.01 **; by two-tailed, unpaired t test.

FIG. 15C shows IImmunochemical analyses of amylin in blood, brain and pancreatic tissues from transgenic and wild-type rats. HC analysis with anti-amylin antibody (brown) on brain sections from HIP rats (n=5/group). Scale bars, 50 μm Data are means±SEM. P≤0.05 *, P≤0.01 **; by two-tailed, unpaired t test.

FIG. 15D shows Immunochemical analyses of amylin in blood, brain and pancreatic tissues from transgenic and wild-type rats. IHC analysis with anti-amylin antibody (brown) on brain sections from HIP and WT rats (n=5/group). Scale bars, 50 μm. Data are means±SEM. P≤0.05 *, P≤0.01 **; by two-tailed, unpaired t test.

FIG. 15E Immunochemical analyses of amylin in blood, brain and pancreatic tissues from transgenic and wild-type rats. shows Images of IHC staining of amylin in HIP rat pancreas (positive control for amylin deposition; Scale bars, 50 μm. Data are means±SEM. P≤0.05*, P≤0.01 **; by two-tailed, unpaired t test.

FIG. 15F Immunochemical analyses of amylin in blood, brain and pancreatic tissues from transgenic and wild-type rats. shows Images of IHC staining of amylin in HIP rat pancreas (positive control for amylin deposition; Scale bars, 50 μm. Data are means±SEM. P≤0.05 *, P≤0.01 **; by two-tailed, unpaired t test.

FIG. 16A shows. Immunochemical analyses of Aβ in brain tissues from transgenic and wild-type rats Images of IHC staining with anti-Aβ antibody (brown) on brain sections from AD rats (n=5 rats/group). Scale bars, 50 μm.

FIG. 16B shows. Immunochemical analyses of Aβ in brain tissues from transgenic and wild-type rats, Images of IHC staining with anti-Aβ antibody (brown) on brain sections from HIP rats (n=5 rats/group). Scale bars, 50 μm

FIG. 16C shows. Immunochemical analyses of Aβ in brain tissues from transgenic and wild-type rats. Images of IHC staining with anti-Aβ antibody (brown) on brain sections from AD WT rats (n=5 rats/group). Scale bars, 50 μm.

FIG. 17 shows. Viability of endothelial cells under amylin-induced stress. Percent cell viability from the MTS assay in ECs treated with various concentrations of human amylin (500 nM, 1 μM, 5 μM, and 10 μM) or vehicle, for 24 hours. Data are mean±SEM. P>0.05; not significant by one-way ANOVA with Dunnett's post-hoc.

FIG. 18A shows. Structural integrity test of the EC monolayer following amylin-induced stress. Diagrammatic representation of the in vitro BBB model (ECs monolayer luminal chamber; astrocytes abluminal chamber) used in testing the structural integrity of the EC monolayer.

FIG. 18B shows At maximum TEER, EC monolayers were treated for 24 hours with vehicle, human amylin (10 μM) or rat amylin (10 μM) (n=5 preparations/group); TEER was measured at different time points (3, 6, 12 and 24 hours). two-way ANOVA with Tukey's post-hoc.

FIG. 18C shows Measurement of the paracellular transport of FITC-Dextran (4 kDa) after the treatments described in (b) two-way ANOVA with Tukey's post-hoc

FIG. 18D shows Measurement of the paracellular transport of FITC-Dextran (4 kDa) permeability coefficients for FITC-Dextran (n=5 preparations/group). Data are mean±SEM. P<0.0001 ****; P>0.05, not significant by one-way ANOVA

FIG. 18E shows Bright field micrographs of ECs after vehicle, human amylin or rat amylin treatments. Scale bars, 100 μm

FIG. 19A shows Test of biochemical properties of amyloid in pancreatic tissue from transgenic rats, Representative images of immunofluorescence staining of human amylin (red) and ThioS (green) in HIP rat pancreas (positive control for amylin amyloid);

FIG. 19B shows Test of biochemical properties of amyloid in pancreatic tissue from transgenic rats, Same as in (FIG. 19A) for AKO rats pancreas (negative control for amylin immunoreactivity signal) Scale bars, 50 μm.

While the disclosure is susceptible to various modifications and alternative forms, specific embodiments thereof have been shown by way of example in the drawings and are herein described below in detail. It should be understood, however, that the description of specific embodiments is not intended to limit the disclosure to cover all modifications, equivalents and alternatives falling within the spirit and scope of the disclosure as defined by the appended claims.

### DESCRIPTION OF EXEMPLARY EMBODIMENTS

The details of one or more embodiments of the presently-disclosed subject matter are set forth in this document. Modifications to embodiments described in this document, and other embodiments, will be evident to those of ordinary skill in the art after a study of the information provided in this document. The information provided in this document, and particularly the specific details of the described exemplary embodiments, is provided primarily for clearness of understanding and no unnecessary limitations are to be understood therefrom. In case of conflict, the specification of this document, including definitions, will control.

While the terms used herein are believed to be well understood by those of ordinary skill in the art, certain definitions are set forth to facilitate explanation of the presently-disclosed subject matter.

Unless defined otherwise, all technical and scientific terms used herein have the same meaning as is commonly understood by one of skill in the art to which the invention(s) belong.

All patents, patent applications, published applications and publications, GenBank sequences, databases, websites and other published materials referred to throughout the entire disclosure herein, unless noted otherwise, are incorporated by reference in their entirety.

Where reference is made to a URL or other such identifier or address, it understood that such identifiers can change and particular information on the internet can come and go, but equivalent information can be found by searching the internet. Reference thereto evidences the availability and public dissemination of such information.

As used herein, the abbreviations for any protective groups, amino acids and other compounds, are, unless indicated otherwise, in accord with their common usage, recognized abbreviations, or the IUPAC-IUB Commission on Biochemical Nomenclature (see, Biochem. (1972) 11(9):1726-1732).

Although any methods, devices, and materials similar or equivalent to those described herein can be used in the practice or testing of the presently-disclosed subject matter, representative methods, devices, and materials are described herein.

Following long-standing patent law convention, the terms “a”, “an”, and “the” refer to “one or more” when used in this application, including the claims. Thus, for example, reference to “a biomarker” includes a plurality of such biomarkers, and so forth.

Unless otherwise indicated, all numbers expressing quantities of ingredients, properties such as reaction conditions, and so forth used in the specification and claims are to be understood as being modified in all instances by the term “about”. Accordingly, unless indicated to the contrary, the numerical parameters set forth in this specification and claims are approximations that can vary depending upon the desired properties sought to be obtained by the presently-disclosed subject matter.

As used herein, the term “about,” when referring to a value or to an amount of mass, weight, time, volume, width, length, height, concentration or percentage is meant to encompass variations of in some embodiments ±10%, in some embodiments ±5%, in some embodiments ±1%, in some embodiments ±0.5%, and in some embodiments ±0.1% from the specified amount, as such variations are appropriate to perform the disclosed method.

As used herein, ranges can be expressed as from “about” one particular value, and/or to “about” another particular value. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, “optional” or “optionally” means that the subsequently described event or circumstance does or does not occur and that the description includes instances where said event or circumstance occurs and instances where it does not. For example, an optionally variant portion means that the portion is variant or non-variant.

As used herein, the term “subject” refers to a target of administration. The subject of the herein disclosed methods can be a mammal. Thus, the subject of the herein disclosed methods can be a human, non-human primate, horse, pig, rabbit, dog, sheep, goat, cow, cat, guinea pig or rodent. The term does not denote a particular age or sex. Thus, adult and newborn subjects, as well as fetuses, whether male or female, are intended to be covered. A “patient” refers to a subject afflicted with a disease or disorder. The term “patient” includes human and veterinary subjects.

As used herein, the term “treatment” or “treating” refers to the medical management of a subject with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

As used herein, the term “prevent” or “preventing” refers to precluding, averting, obviating, forestalling, stopping, or hindering something from happening, especially by advance action. It is understood that where reduce, inhibit or prevent are used herein, unless specifically indicated otherwise, the use of the other two words is also expressly disclosed. As will be understood by those of ordinary skill in the art, when the term “prevent” or “prevention” is used in connection with a prophylactic treatment, it should not be understood as an absolute term that would preclude any modicum of pain in a subject. Rather, as used in the context of prophylactic treatment, the term “prevent” can refer to inhibiting the development of or limiting the severity of, arresting the development of pain, and the like.

As used herein, the terms “administering” and “administration” refer to any method of providing a pharmaceutical preparation to a subject. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, and parenteral administration, including injectable such as intravenous administration, intra-arterial administration, intramuscular administration, and subcutaneous administration. Administration can be continuous or intermittent. In various aspects, a preparation can be administered therapeutically; that is, administered to treat an existing disease or condition. In further various aspects, a preparation can be administered prophylactically; that is, administered for prevention of a disease or condition.

As used herein, the term “neurological disease or deficiency” refers broadly to diseases of the nervous system including the brain, spinal cord, and nerves. Neurological disease or deficiency may include for example: hypoxic-ischemic brain injury, Alzheimer's Disease, behavioral deficits, brain microhemorrhages, or axonal degeneration.

As used herein, the term “neurological deficits” refers broadly to deficiencies with neurological function. Neurological deficits may refer to a reduction or loss of a behavior or skill as compared to normal subjects. Neurological deficits may occur in balancing ability, motor coordination, reaction time, speed, short-term memory recognition, memory recall, and the like. Deficits in these abilities are readily ascertainable by those skilled in the art.

The term “effective amount” refers to an amount that is sufficient to achieve the desired result or to have an effect on an undesired condition. For example, a “therapeutically effective amount” refers to an amount that is sufficient to achieve the desired therapeutic result or to have an effect on undesired symptoms, but is generally insufficient to cause adverse side effects. The specific therapeutically effective dose level if or any particular patient will depend upon a variety of factors including the disorder being treated and the severity of the disorder; the specific composition employed; the age, bodyweight, general health, sex and diet of the patient; the time of administration; the route of administration; the rate of excretion of the specific compound employed; the duration of the treatment; drugs used in combination or coincidental with the specific compound employed and like factors well known in the medical arts. For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. Dosage can vary, and can be administered in one or more dose administrations daily, for one or several days. Guidance can be found in the literature for appropriate dosages for given classes of pharmaceutical products.

### EXAMPLES

### MATERIALS & METHODS

Human samples: The protocol concerning the use of biopsy from patients was approved in agreement with Institutional Review Board approval and informed consent was obtained prospectively. Human brain tissues and cerebrospinal fluid (CSF) samples were used in this study. Brain sections from familial Alzheimer's disease (fAD) patients, and age-matched cognitively normal (CN) individuals (temporal cortex areas) were provided by Queen College of London, United Kingdom. Frozen brain tissue and sections from fAD patients (temporal cortex areas) were provided by King's College of London, United Kingdom. Brain tissues from sporadic AD patients (sAD) and age-matched CN individuals (Brodmann areas 9 and 21/22) were provided by the Alzheimer's Disease Center at the University of Kentucky, USA. Brain samples from CN individuals were used as controls. Frozen brain tissues from fAD patients and controls were used for biochemical analyses. For immunohistochemistry, formalin fixed, paraffin embedded brain tissues from sAD patients, fAD patients and age-matched controls were used. CSF samples from AD patients and CN individuals were provided by the University of Gothenburg, Sweden. CSF samples from patients with mild cognitive impairment (MCI) and from CN individuals were provided by the University of Kentucky, University of Washington and Wake Forest University, USA. Data on CSF Aβ₄₂ were provided by the study centers. CSF Aβ levels in samples from the University of Gothenburg, University of Kentucky, University of Washington, and Wake Forest University were measured with the INNO-BIA AlzBio3 multiplex assay (FujiRebio). Details on patient information can be found in Table 5.

TABLE 3
Neuropathological information, age and sex for individuals with normal cognitive
(control), familial Alzheimer’s disease (AD), sporadic AD and mild cognitive impairment
(MCI) in the present study.

| | Control | Familial AD | Sporadic AD |
|---|---|---|---|
| Histological analyses | n = 5 | n = 27 | n = 6 |
| Gender, female/male (% female) | | 17/10 (63%) | 4/6 (67%) |
| Age at death (avg ± SEM) | | 54.2 ± 1.8 | 89.8 ± 2 |
| Braak stage | N.A. | V-VI | |
| Mutation, PSEN1/APP | N.A. | 20/7 | N.A. |
| | Control | Familial AD | |
| Amylin ELISA | n = 23 | n = 18 | |
| Gender, female/male (% female) | | 12/18 (67%) | |
| Age at death (avg ± SEM) | | 54 ± 2.4 | |
| Braak stage | N.A. | V-VI | |
| Mutation, PSEN1/APP | N.A. | 11/7 | N.A. |
| | Control | Sporadic AD | MCI |
| CSF samples | n = 119 | n = 36 | n = 70 |
| Gender, female/male (% female) | 54/65 (45%) | 21/15 (58.3%) | 30/40 (42.9%) |
| Age at collection (avg ± SEM) | 70.3 ± 0.8 | 77.6 ± 1.2 | 69.6 ± 1.3 |

Genetic analysis. The specific association of genetic variants in LAPP identified by the International Genomics of Alzheimer's Project (IGAP) consortium was analyzed. These results correspond to the meta-analysis of genotyped and imputed data (7,055,881 SNPs, 1000G phase 1 alpha imputation, Build 37, Assembly Hg19) of 17,008 Alzheimer's disease cases and 37,154 controls. To assess rare genetic variants in IAPP (ENST00000240652) in Alzheimer's disease, the exome sequencing data from a UK cohort of 331 AD cases was analyzed. The variability of the IAPP gene in a cohort of healthy elderly samples from the Healthy Exomes (HEX) database was also analyzed. HEX includes data corresponding to the exome sequencing of from 468 individuals categorized as cognitively healthy and neuropathologically normal [REF Guerreiro]. Given the finding of p.Asn64fs in a healthy sample aged >90 years, loss of function variants described in gnomAD and the respective available information for age was examined (Tables 2 and 3).

Experimental animals: This investigation conforms to the Guide for the Care and Use of Laboratory Animals published by the US National Institutes of Health (NIH Publication No. 85-23, revised 2011) and was approved by the Institutional Animal Care and Use Committees at University of Kentucky. Alzheimer's disease (AD) rats (TgF344-19, provided by Charles River Laboratory) are Fischer rats that express human Aβ (A4) precursor protein (hAPP) gene with the Swedish mutation (K595N/M596L), and presenilin 1 (PSEN1) gene with a deletion of exon 9, driven by mouse prion promoter (Prp)²⁸. HIP rats (provided by Charles River Laboratory) are Sprague-Dawley rats that overexpress (3-fold) human amylin in the pancreatic β-cells²⁹. The AD rats were crossbred with HIP rats to generate rats that are triple transgenic for human amylin, APP, and PSEN1 (ADHIP rat). ADHIP, AD and wild-type (WT) (n=17/group; n=51 rats in total) were used in behavior testing and physiological analyses. Amylin knock-out in AD model (AD-AKO) was generated by crossbreeding AD rats with AKO rats (the generation of AKO rat model was described previously¹⁵). AD and AD-AKO littermates (n=6/group; n=12 rats in total) were used for behavior testing and physiological analyses. Blood glucose and weights were measured monthly in all rats.

Antibodies and reagents The following primary antibodies were used: Amylin (1:200, T-4157, Bachem-Peninsula Laboratories), human Aβ (1:300, clone 6E10, Biolegend), Ibal (1:300, 019-19741, Wako), CD68 (1:200, MCA341GA, Biorad), myelin basic protein (1:5,000, AMAB91064, clone CL2829, Sigma), phosphorylated Tau (1:400, clone ATB, MN1020, Pierce). The following secondary antibodies were used: Biotinylated anti-mouse IgG (1:300, BA-2000, Vector), anti-rabbit IgG (1:300, BA-1100, Vector), AP-conjugated anti-mouse IgG (1:100, A3562, Sigma), Alexa Fluor 568 anti-rabbit IgG plus (A11036) and Alexa Fluor 647 anti-mouse IgG plus (A21236), Alexa Fluor 488 anti-mouse IgG (A11029) and Alexa Fluor 488 anti-rabbit IgG (A11034) from ThermoFisher. The following reagents were used: DAB (3,3′-diaminobenzidine tetrahydrochloride) chromogen substrate (ab64238, Abcam), AEC (3-amino-9-ethylcarbazole) chromogen substrate (SK-4200, Vector), StayGreen/AP chromogen substrate (ab156428, Abcam), Luxol fast blue dye (AC212170250, Acros Organics), potassium ferrocyanide (AC211095000, Acros Organics), Congo Red (C580-25, Fisher), citrate buffer (S1699, Dako), Thioflavin S (1326-12-1, Sigma), Sudan black (4197-25-5, Sigma), lyophilized amidated human amylin peptide (AS-64451-05, Anaspec), BCA (23225, ThermoFisher) and Micro-BCA (23235, ThermoFisher) protein assays, DNA purification kit (K0512, ThermoFisher), Sybr green qPCR mix (1725150, Biorad). Primers were from IDT.

Assessment on animal health Animals in study groups (n=17/group) were followed longitudinally. Animals were considered unhealthy when animals display any sign of lethargy, sarcopenia, respiratory distress, gait abnormality or dehydration. Percentage of healthy animals vs age was then analyzed using Log-rank test.

Bio-fluids collection from animals Bio-fluids were collected from animals every two months. The collection was performed in isoflurane-anesthetized animals. CSF was collected by inserting needles through the cisterna magna without making any incision at this region. Protocol was described in Ref 30. CSF was drawn by simple syringe aspiration. The yielded fluid volume did not exceed 120 μL per each collection. Blood was collected by inserting needles through the tail vein. Blood was drawn by simple syringe aspiration. EDTA was added to blood samples to prevent coagulation. The collection volume did not exceed 500 μL per each collection. Red blood cells and plasma were separated by centrifugation at 1,000×g for 10 minutes at 4° C. Samples were stored in −80° C.

Pharmacological treatment on animals To increase plasma eicosanoids levels, the animals were treated with TPPU (1-Trifluoromethoxyphenyl-3-(1-propionylpiperidin-4-yl) urea, N-[1-(1-Oxopropyl)-4-piperidinyl]-N′-[4-(trifluoromethoxy)phenyl]-urea), a soluble epoxide hydrolase inhibitor (sEHi) (HY-101294, MedChem Express). Treatment with sEHi has been shown to lower the level of circulating amylin²⁴. 12 months old (n=5; early-stage) and 16 months old (n=6; late stage) animals were subjected to treatment with sEHi. For treatment at early-stage, the drug was administered through drinking water (3 mg/L, daily) and intravenous injection (20 μg/kg, once a week) to ensure equal intake for each animal. For treatment at late-stage, the drug was administered through and intravenous injection (20 μg/kg, daily, 1 month) and then through drinking water (3 mg/L, daily, 1 month). Amount of water intake was measured daily.

Behavior testing Forelimb use test/Cylinder test, inclined plane, hind limb clasping test. The protocol for these behavioral tests were described previously¹⁵. Animal forelimb deficit was evaluated by forelimb-to-wall contact time in cylinder test. Animal balancing ability was tested by the angle at which the animal started to free-fall on the raising inclined plane. Abnormalities in the animal hind limbs were assessed by scoring the severity of hind limb clasping.

Rotarod assessment Motor coordination and balance were tested by the rotarod (Rotamex 5, Columbus Instrument, OH) test²⁷. Animals were acclimatized to the static rod 2 days prior to testing. On the testing day, the speed of the rotarod was increased from 0 rpm to 40 rpm within 2 minutes. Each rat was tested on the rotarod for a total of 4 trials per day over 5 consecutive days. For each training day, the smallest value of latency-to-fall for each rat was discarded. The remaining read-outs were averaged, and a group average was calculated for each genotype.

Novel Object Recognition (NOR) The NOR test was used to test for short-term recognition, as previously described²⁷.

Morris Water Maze Spatial learning and long-term memory retention were tested in a 1.5 m diameter Morris Water Maze, as previously described¹⁵. Animals were given 4 learning trials per day for four consecutive days using random starting locations. Animals were allowed to stand on the platform for 30 seconds after the first trial and 15 seconds after each additional trial. If the animals failed to locate the platform, they were picked up and put on the platform for 15 seconds. To assess reference memory, a probe trial was given 24 hours after the fourth acquisition day. Trials were recorded by EthoVision XT software (Noldus, VA).

Composite z-score analysis for behavior tests The composite z-score analysis method was previously described³¹. For each behavior test, mean and standard deviation were calculated from individual variables collected at 12 months and 16 months old animals across the experimental groups. Z-score for each animal in each behavior test was calculated using the following equation:

$$z - score = \frac{individual variable - mean}{standard deviation}$$

The composite score for each animal was calculated by averaging z-score from each behavior test.

Histology and demyelination scoring Myelination in rat brains was analyzed by staining with Luxol fast blue (LFB) dye. Scoring analysis method was performed as described previously¹⁵. Microhemorrhages in rat brains were stained with Prussian blue dye and analyzed as described previously¹⁵. Congo red staining was performed on the human brains as previously¹¹ described.

Immunohistochemistry. Formalin fixed, paraffin embedded brain and pancreas tissues from humans, ADHIP, AD, WT, and AD-AKO rats were used. Tissues were processed as previously described^{11,14,15,27}. After tissue rehydration, the endogenous peroxidase was quenched in 3% H₂O₂ in methanol for 30 minutes. For amylin and Aβ antigen retrieval, sections were treated with 100% formic acid for 5 minutes, followed by 0.5% pepsin digestion in 5 mM HCl for 20 minutes at 37° C. To retrieve intra-neuronal amylin antigen (rat brains) as well as other antigens, tissue sections were heated in citrate buffer for 30 minutes. Non-specific antibody binding was blocked by 15% horse serum for 1 hour at room temperature (RT). Primary antibodies against amylin, human Aβ, Iba1, CD68 or myelin basic protein (MBP) was incubated on slides overnight at 4° C. Sections were then washed and incubated with secondary antibodies. Signal was developed with DAB or AEC peroxidase substrate. For co-staining with two antibodies, after the signal was developed for the first antibody, sections were then rinsed in water. Non-specific antibody binding was blocked with 10% normal goat serum, and the sections were incubated with the second primary antibody overnight at 4° C. Sections were then washed and incubated with AP-conjugated secondary antibody, and developed with StayGreen/AP chromogen substrate. Sections were mounted with aqueous mounting medium. The specificity of the amylin antibody in both human and rat brain tissues was established in previous studies^{11,14,15,27}.

Imaging analysis Wide-field images of stained tissue sections were generated by stitching images obtained from the 10× objective lens (Nikon NIS-Element Software). Higher magnification images for specific tissue area were obtained using the 40× objective lens. The immunoreactivity signal for each antibody was analyzed by ImageJ. Clearly defined-signal pixels were selected to establish the RGB profile of the color of interest. The threshold for each color signal was adjusted to reduce background noise. The established RGB profile and threshold were applied to a Macro script command, using Color Deconvolution plugin in ImageJ. The staining area was calculated using the following equation:

$$Staining area ( {μ m}^{2} ) = \\ ( \frac{Average \% Pixels Area readout}{100} ⨯ Imaging pixels area ) ⨯ {μ m}^{2} per {pixel}^{2}$$

The imaging pixels area is 1280×1024. μm² per pixels² is 0.84 for 10× objective lens and 0.05 for 40× objective lens. The staining area (μm²) was normalized to the total area of the tissue section.

$$Normalized immunoreactivity signal = ( \frac{Staining area ( {μ m}^{2} )}{Total area of tissue section ( {μ m}^{2} )} )$$

Immunofluorescence staining Immunofluorescence staining for brain tissue sections was previously described^{14,15} with modifications. Antigen retrieval for amylin and Aβ was described in the immunohistochemistry session above. Primary antibodies are amylin, human Aβ and phosphorylated Tau. For Thioflavin S staining, after secondary antibody incubation, slides were incubated in 0.5% Thioflavin S for 15 minutes at room temperature. Slides were then incubated for 3 minutes in 70% ethanol, 3 minutes in 0.2% Sudan black and 3 minutes in 70% ethanol, before washing and mounting.

Magnetic resonance imaging (MRI) MRI scans were performed on ADHIP, AD and WT littermate rats using a horizontal 7T nuclear MRI scanner (ClinScan, Brucker BioSpin MRI, Ettlingen, Germany) as previously described¹⁵. Coronal T2-weighted images were obtained using generic parameters: field of view (FOV) 40 mm, repetition time (TR) 3000 ms, echo time (TE) 24 ms, slice thickness 1 mm, inter-slice gap 1 mm, 7 slices. Ventricular hyperintensities volume was calculated by the method described previously¹⁵.

Amylin aggregation and injection Lyophilized amidated human amylin peptide was dissolved in PBS pH 7.4 to the concentration of 50 μM. The mixture was incubated in 37° C. for 72 hours with occasional shaking to allow amylin to form aggregates. Every 3 days, aggregated human amylin solution was injected into 7 months old AD rat via tail vein (60 μg/kg). The age-matched AD control group received the same volume of PBS per injection without aggregated human amylin. The animals received injections for 60 days. Bio-fluids from each animal were collected before- and post-injection.

Isolation of rat brain capillaries Rat brain capillaries were isolated following the protocol described previously¹⁵. For quality control, capillaries were stained with Texas red dye and were examined under the confocal microscope. Freshly isolated brain was snapped frozen, crushed and homogenized in homogenate buffer (150 mM NaCl, 50 mM Tris-HCl, 50 mM NaF, 2% Triton X-100, 0.1% SDS, 1% (v/v) protease and phosphatase inhibitors, pH 7.5). Homogenates were centrifuged at 17,000×g for 30 minutes at 4° C. The supernatant was separated from pellet after centrifugation and were then used for all experiments.

Protein extraction Frozen human brain tissues were homogenized in homogenate buffer (150 mM NaCl, 50 mM Tris-HCl, 50 mM NaF, 2% Triton X-100, 0.1% SDS, 1% (v/v) protease and phosphatase inhibitors, pH 7.5). Homogenates were centrifuged at 17,000×g for 30 minutes at 4° C. The supernatant was separated from pellet after centrifugation and was then used for all experiments. For rat brain tissue, half hemisphere was used for histological analyses, and the other half was used for brain capillary isolation and other protein extractions. Rat brain tissues were subjected to serial extraction method. Frozen brain samples were homogenized with 1% Triton buffer (25 times tissue volume) containing 20 mM Tris, 150 mM NaCl, 1 mM EDTA, 1 mM EGTA, 1% Triton X-100 (v/v), 1% (v/v) protease and phosphatase inhibitors, pH 7.5. The homogenates were left on ice for 15 minutes. The homogenates were centrifuged at 15,000 rpm for 15 minutes at 4° C. The supernatant (Triton-soluble fraction) was separated from the pellet. 5 M Guanidine HCl (with 50 mM Tris, pH 8.0) solution was added to the pellet (10 times the pellet volume). Homogenates were rocked for 3-4 hours at room temperature. The samples were stored at −80° C. until analysis. Before analysis, the Guanidine HCl (GuHCl) samples were diluted at 1:10 ratio with lysate buffer (1% NP-40 (v/v), 150 mM NaCl, 10 mM Tris, 2 mM EGTA, 50 mM NaF). The samples were centrifuged at 16,000×g for 20 minutes at 4° C. The supernatant (GuHCl-soluble fraction) was separated from the pellet. BCA protein estimation was performed for Triton-soluble fractions. MicroBCA protein estimation was performed for GuHCl fractions.

Enzyme-linked immune absorbance assay (ELISA) Levels of amylin in human brain samples were measured using sandwich amylin ELISA from Millipore (EZHA-52K). Levels of amylin in human CSF and animal samples were measured using amylin sandwich ELISA from R&D system (EIA-AMY). Aβ levels in animal CSF were measured using high sensitivity electrochemiluminescence ELISA (MSD 6E10, K15200G-2, Meso Scale Discovery). Hypoxia-inducible transcription factor 1α and hypoxia-inducible transcription factor 2α (MBS764727, MBS2601406, MyBioSource), arginase-1 and arginase-2 (MBS289817, MBS7216305 MyBioSource), rat erythropoietin (EPO) ELISA (442807, Biolegend) and VCAM-1 (LS-F24285; LS Bio) were performed according to the manufacturers protocol.

Arginase activity Arginase activity in rat brain capillaries were measured using arginase activity kit (MAK112, Sigma). Experimental protocol and analysis were performed according to manufacture instruction.

Mitochondria DNA extraction and analysis Protocol for quantifying mitochondrial DNA content was described in Ref 32. DNA was extracted from frozen rat brain tissues using genomic DNA purification kit, following manufactures protocol. DNA purity was assessed by ensuring the A₂₆₀/A₂₈₀ ratio was >1.8. Mitochondrial DNA (mtDNA) content was measured using Sybr green based real-time (RT) qPCR. The primers were specific for the regions of mitochondrial gene 16S rRNA (forward: 5′-TCCCAATGGTGCAGAAGCTATTA-3′(SEQ ID NO: 1); reverse: 5′-AAGGAGGCTCCATTTCTCTTGTC-3′(SEQ ID NO: 2). House-keeping gene primers were specific for beta actin (forward: 5′-CTAAAGGTGACCAATGCTGGAGG-3′(SEQ ID NO: 3); reverse: 5′-TGGCATAGAGGTCTTTACGGATG-3′(SEQ ID NO: 4)). 3. The RT-qPCR thermocycling conditions were 3 minutes at 98° C., 30 seconds at 95° C., and 40 cycles of 30 seconds denaturation at 95° C., 30 seconds annealing at 60° C. and 30 seconds extension at 72° C. The fluorescence signal intensities of the PCR products were recorded in Biorad CFX96 RT-qPCR system. Final data was analyzed with Biorad CFX manager 2.1 software and Excel. The relative mtDNA copy number was calculated as the difference in the numbers of threshold cycles (Cq) between the nuclear gene and the mtDNA gene (ACq), in which the amount of mtDNA was calculated per cell, 2(2-ΔCq), accounts for the 2 beta actin copies in each cell nucleus.

Statistical analysis Gaussian distribution of the data was tested with D′Agostino-Pearson and Kolmogorov-Smirnov test. Parametric comparison between two groups was performed using two-tailed Student's t tests. Non-parametric comparisons between two groups was performed using Mann-Whitney U-tests. Relationships between two variables was analyzed by correlation analysis. Z-scores and data from the Morris Water Maze and Rotarod were analyzed by repeated measures analysis. All models were linear regression with a first-order autoregressive (AR) covariance structure, except for Rotarod for age 12M AD vs ADHIP, where the AR structure did not converge and the simpler compound symmetry structure was fit instead. All models included main effects for group (i.e., genotype or treatment) and trial day or age, as well as a cross-product interaction between group and day (or age) to test for differences in the slope of the group learning curves. Post-hoc tests were performed to assess group differences on each testing day. Repeated analyses were performed using SAS 9.4® PROC MIXED (SAS Institute, Inc.; Cary, N.C.; USA). Data are presented as individuals, means±SEM or means. Difference between groups was considered significant when P<0.05. All other analyses were performed using GraphPad Prism 5.0 software.

### RESULTS

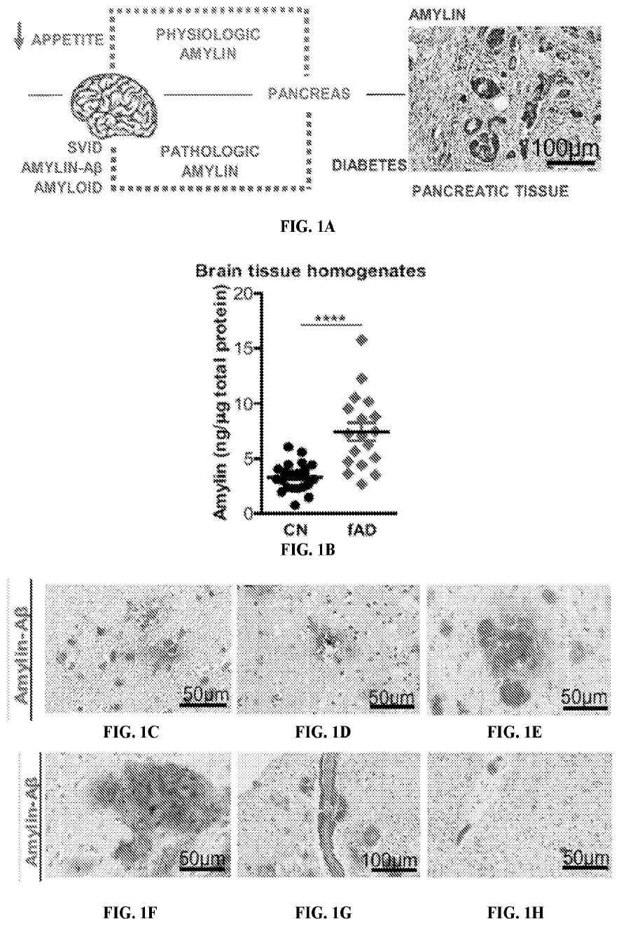

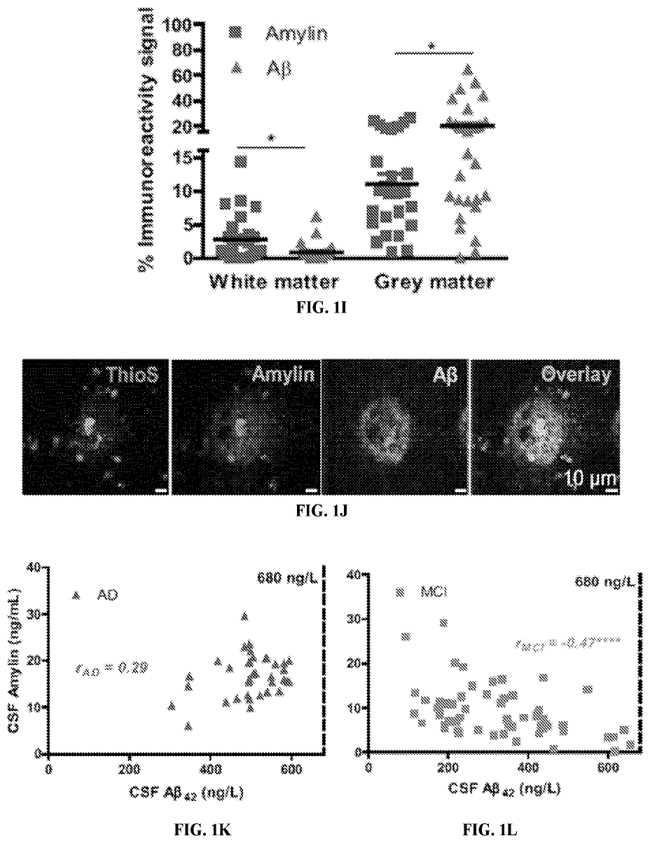

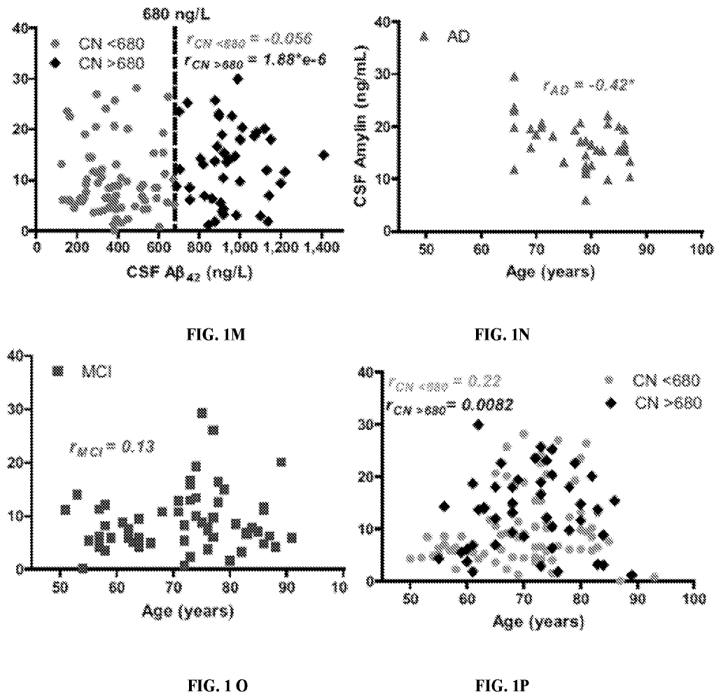

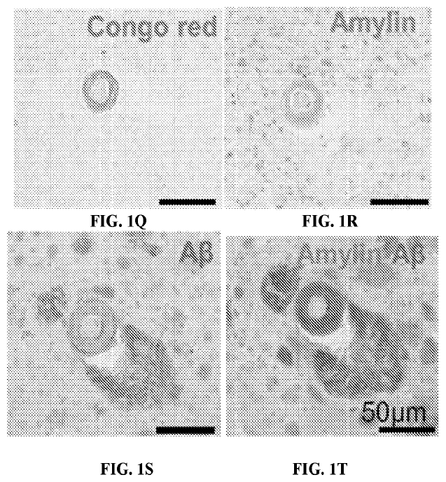

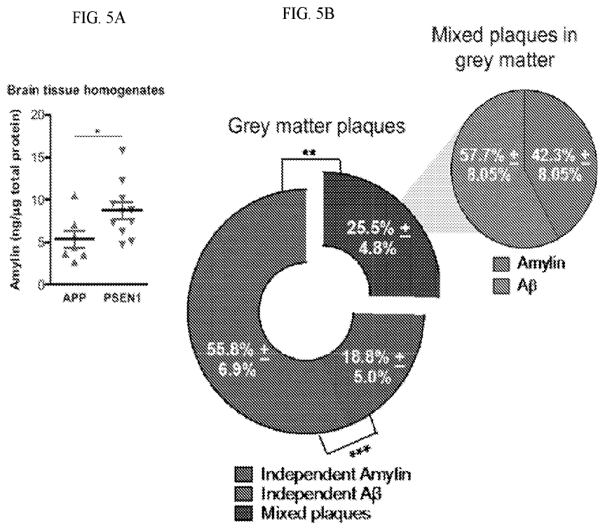

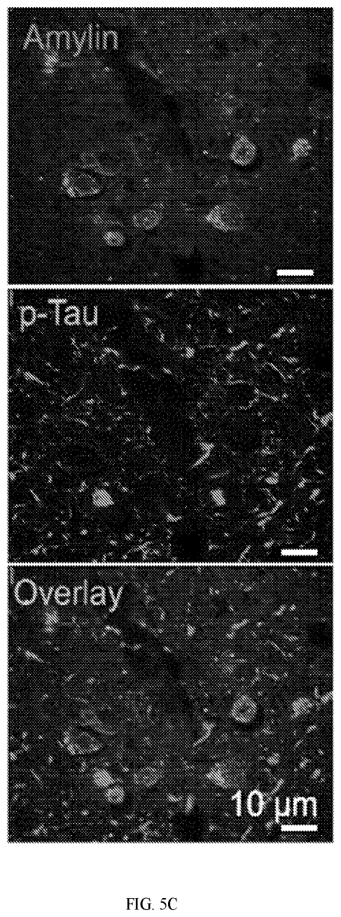

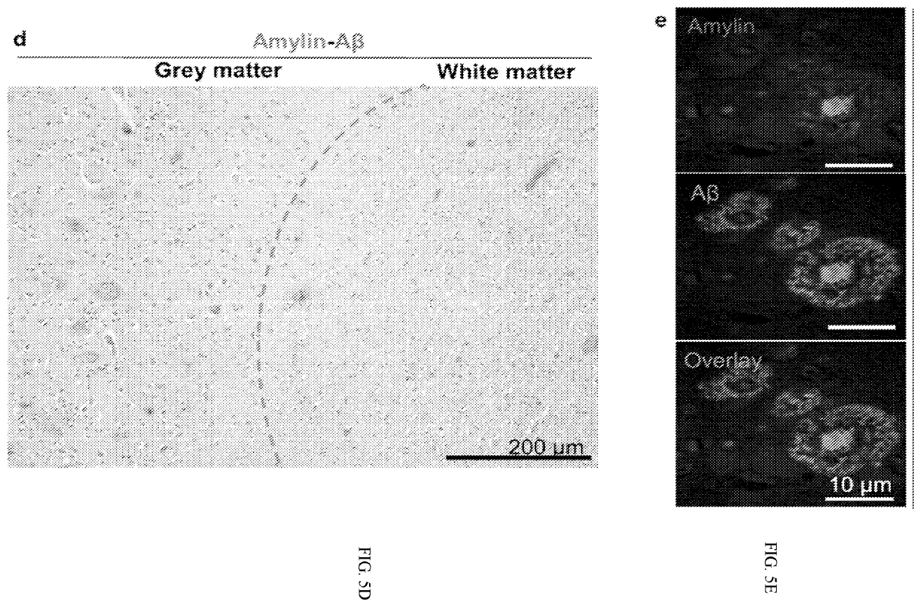

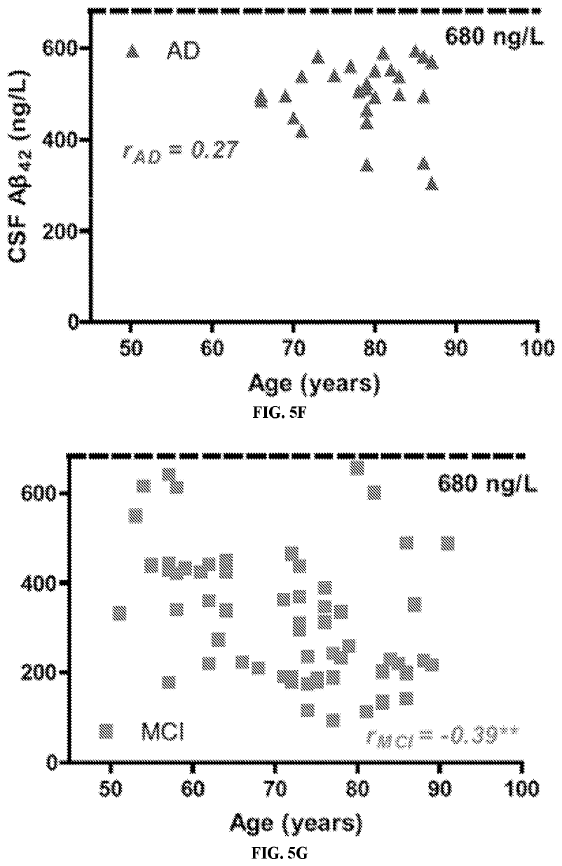

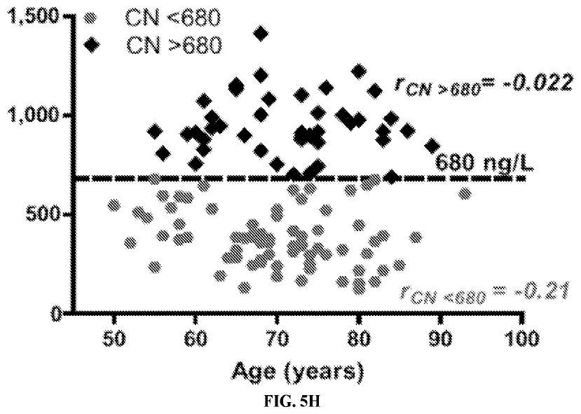

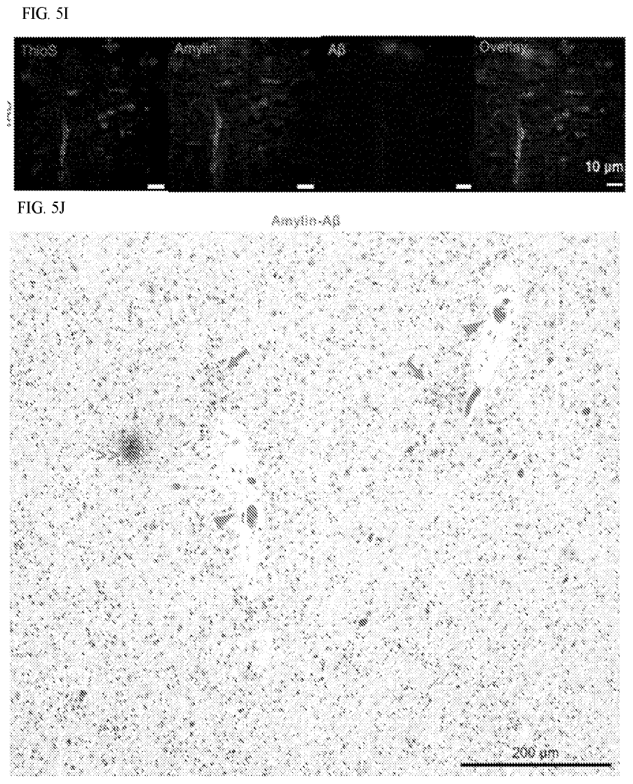

Example 1: Brain tissues from PSEN1 and APP mutation carriers were investigated for amylin deposition and interaction with AD pathology. Temporal cortex homogenates from fAD brains had higher amylin concentrations, compared to the cognitively normal (CN) group (FIG. 1b; FIG. 5a). Amylin immunoreactivity was detected within neuronal soma (FIG. 1c; 26/27 patients; see FIG. 5b and Table 1 for overall pathology distribution), neuritic plaques (FIG. 1d; 16/27 patients), amylin-Aβ plaques (17/27 patients) that have layered (FIG. 1e) and mixed (FIG. 1f) compositions, and small blood vessels, both in the grey matter (GM; FIG. 1g) and white matter (WM; FIG. 1h) regions. Amylin-associated pathology appeared higher in WM vs. GM regions (FIG. 1j; FIG. 5d).

TABLE 1
Number of familial AD patients with different brain amylin and Aβ
pathology, assessed by immunohistochemical analysis. Total number
of human brains analyzed is 27.

| Familial AD brain pathology | Patients/total (%) |
|---|---|
| Mixed amylin-Aβ plaques in tissue parenchyma | 17/27 (63%) |
| Mixed amylin-Aβ plaques in blood vessels | 22/27 (81%) |
| Amylin plaques | 16/27 (59%) |
| Amylin-positive neurons | 26/27 (96%) |

In neuritic plaques, immunostaining showed the presence of amylin in small proteinaceous fragments (FIG. 1c) that appear to be derived from degenerating neurons. Confocal microscopic analysis of areas containing amylin-positive neurons revealed distinct immunoreactivity signals for amylin and p-tau with amylin localized in the soma and cellular membranes (FIG. 5c). Triple staining of fAD brain tissues with Thioflavin S and anti-amylin and anti-Aβ antibodies indicated that the amylin-positive core of mixed amylin-Aβ plaques has biochemical characteristics of amyloid (FIG. 1i).

Analyses of the association of common and rare amylin variants with the risk of developing AD revealed no statistically significant results (Tables 2 and 3).

TABLE 2
Loss of function variants in IAPP present in gnomAD and respective ages.

| #CHR | Position | rsID | Ref. | Alt. | Consequence | Protein Consequence |
|---|---|---|---|---|---|---|
| 12 | 21526335 | rs904381884 | T | A | p.Leu17Ter | p.Leu17Ter |
| 12 | 21526351 | rs62871062 | T | T | p.Pro24HisfsTer27 | p.Pro24HisfsTer27 |
| | | | | A | | |
| | | | | C | | |
| 12 | 21526366 | rs1185850731 | G | C | c.80 + 1G > C | |
| 12 | 21526367 | rs138036034 | T | C | c.80 + 2T > C | |

| #CHR | Trans. Cons. | Annotation | AC | Allele # | Allele Freq. | Ages |
|---|---|---|---|---|---|---|
| 12 | c.50T > A | Stop | 4 | 282632 | 1.42E−05 | 45-50; |
| | | gained | | | | 70-75* |
| 12 | c.69_70dupAC | Frameshift | 1 | 251190 | 3.98E−06 | 55-60 |
| | | variant | | | | |
| 12 | c.80 + 1G > C | Splice | 1 | 251128 | 3.98E−06 | 30-35 |
| | | donor | | | | |
| | | variant | | | | |
| 12 | c.80 + 2T > C | Splice | 44 | 282518 | 1.56E−04 | <30-75 |
| | | donor | | | | |
| | | variant | | | | |

Loss of function variants with a consequence in the canonical transcript of IAPP and without quality flags in gnomAD.
*Age information available for 2 individuals.
#CHR: Chromosome;
Ref.: Reference;
Alt.: Alternate;
Trans. Cons.: Transcript consequence.
AC: Allele count;
Allele Freq.: Allele frequency.

Mixed amylin-Aβ plaques with amylin amyloid-positive cores were identified also in brain tissues from patients with sAD (FIG. 5e). In humans with dementia and AD pathologic change defined by CSF Aβ₄₂<680 ng/L¹⁹, low CSF Aβ₄₂ correlated with low CSF amylin levels (FIG. 1k). In the mild cognitive impairment (MCI) stage of AD, however, CSF amylin and Aβ₄₂ levels were inversely correlated, with lower Aβ₄₂ levels corresponding to higher amylin levels (FIG. 1l). In the CN group, the CSF amylin and Aβ₄₂ levels were variable (FIG. 1m). With increasing age, CSF amylin levels decreased in AD (FIG. 1n) and increased in MCI (FIG. 1o) and CN (FIG. 1p) groups, particularly in humans with CSF Aβ₄₂<680 ng/L (FIG. 1p; green dots).

TABLE 3
Association results from the IGAP consortium for IAPP variants.

| | | | Effect | Non-effect | | | |
|---|---|---|---|---|---|---|---|
| #CHR | POS | MarkerName | allele | allele | Beta | SE | P |
| 12 | 21526443 | rs41275208 | A | C | −0.0448 | 0.0362 | 0.2159 |
| 12 | 21526472 | rs12306121 | G | A | −0.044 | 0.0197 | 0.02536 |
| 12 | 21526651 | rs12319824 | A | G | −0.0425 | 0.0199 | 0.0326 |
| 12 | 21526883 | rs12300126 | C | T | −0.0421 | 0.0203 | 0.03793 |
| 12 | 21527044 | rs73080823 | T | C | −0.0452 | 0.0362 | 0.2124 |
| 12 | 21527681 | rs17680758 | G | T | −0.0319 | 0.0314 | 0.309 |
| 12 | 21527955 | rs12308285 | T | A | −0.0352 | 0.0193 | 0.06842 |
| 12 | 21528337 | rs12811082 | T | G | −0.0415 | 0.0217 | 0.05627 |
| 12 | 21529150 | rs17680787 | T | G | −0.0437 | 0.0208 | 0.03531 |
| 12 | 21529212 | rs12305367 | C | T | −0.0507 | 0.0201 | 0.01168 |
| 12 | 21529687 | rs34417126 | C | A | −0.046 | 0.0204 | 0.02398 |
| 12 | 21529882 | rs34996992 | C | G | −0.0476 | 0.0203 | 0.01902 |
| 12 | 21530485 | rs78331403 | A | G | −0.0405 | 0.0365 | 0.2661 |
| 12 | 21531437 | rs5484 | T | C | −0.0437 | 0.0205 | 0.0333 |
| 12 | 21532100 | rs5486 | G | A | −0.0454 | 0.0361 | 0.2087 |
| 12 | 21532138 | rs1056007 | T | G | −0.0471 | 0.0209 | 0.02439 |
| 12 | 21532191 | rs5487 | C | T | 0.1076 | 0.0579 | 0.06315 |
| 12 | 21532217 | rs5488 | A | T | −0.0515 | 0.0201 | 0.01039 |
| 12 | 21532459 | rs3213208 | G | T | −0.0358 | 0.0383 | 0.3504 |
| 12 | 21532547 | rs12826421 | C | G | −0.0474 | 0.0199 | 0.01742 |

#CHR: Chromosome;
POS: position of the SNP (Build 37, Assembly Hg19);
MarkerName: SNP rsID;
Effect_allele: reference allele (coded allele);
Non_Effect_allele: non reference allele;
Beta: overall estimated effect size for the effect allele;
SE: overall standard error for effect size estimate;
P: meta-analysis P value using regression coefficients (beta and standard error)

Vascular amylin deposition appeared to coincide with cerebral amyloid angiopathy (CAA; FIG. 1r). The triple-stained brain sections for amylin, Aβ and Thioflavin-S showed small vessels positive for amylin and Thioflavin-S, but negative in Aβ, reflecting biochemical characteristics of amylin amyloid (FIG. 5i). Amylin immunoreactivity was also detected in occluded small vessels, chronic infarcts and perivascular areas (FIG. 5j), similar to the vascular amylin pathology found in sAD brains^{11,12,15}.

The results suggest that amylin secreted from the pancreas may modulate brain amyloid composition and contribute to small vessel disease in both familial and sporadic forms of AD.

Example 2: To assess the interaction between amylin dyshomeostasis and AD pathology, a combination of AD rat models, including AD rats expressing non-amyloidogenic rat amylin and AD rats expressing human amylin in the pancreatic β-cells (ADHIP rats) was used. As the negative control for amylin, AD rats with deleted amylin gene (AD-AKO rats), which were generated by crossing AD rats with amylin knockout (AKO) rats were used.

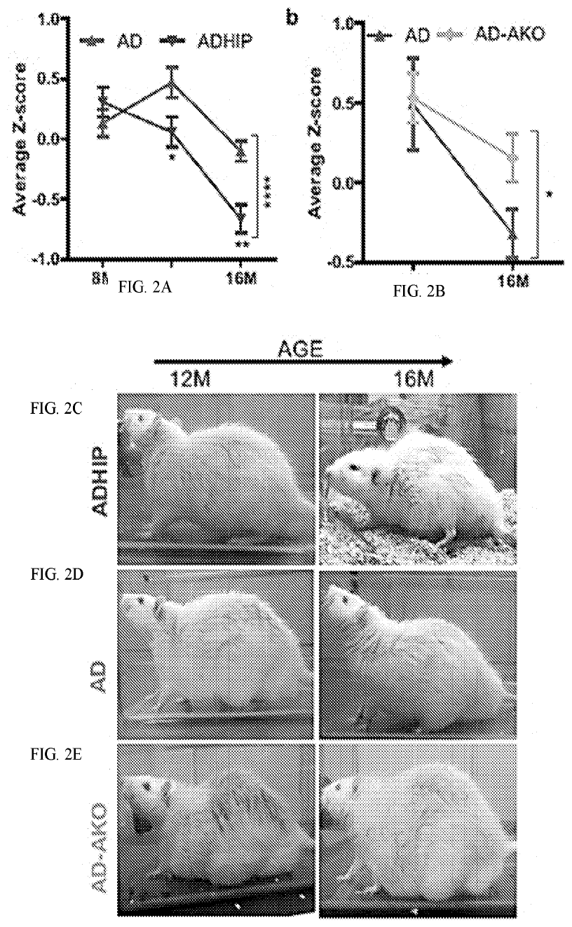

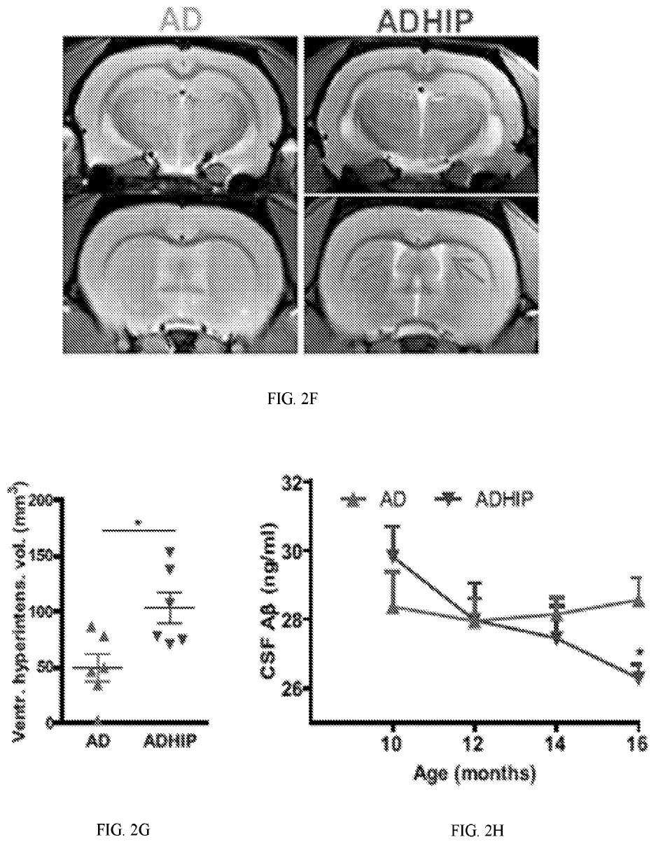

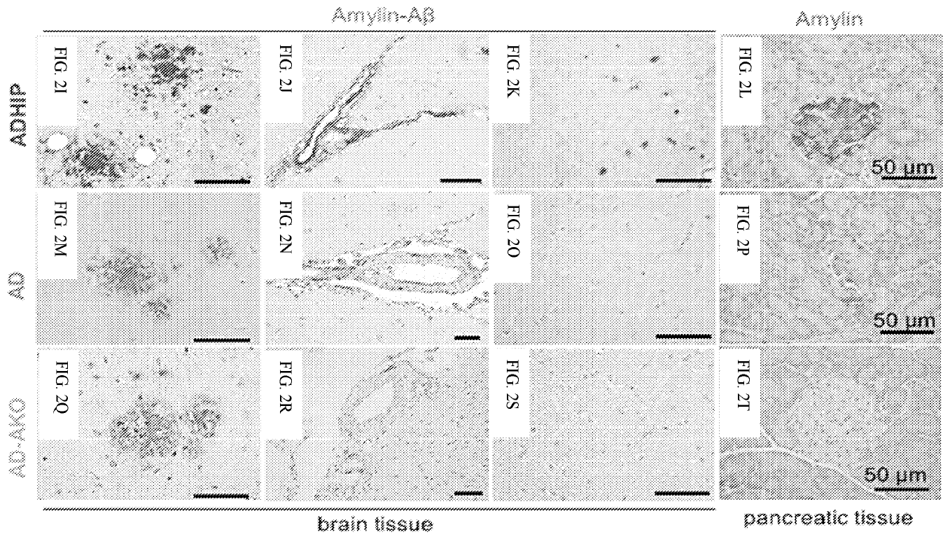

Compared to AD rats, ADHIP littermates had greater motor and cognitive deficits (FIG. 2a, FIGS. 6a-f). AD-AKO rats increased their body weights in time, more than AD littermates (FIG. 6g), consistent with the role of amylin in regulating satiety⁶; however, behavioral changes were ameliorated in aged AD-AKO rats compared to AD littermates (FIG. 2b, FIGS. 6h-j), an unanticipated result. Both overexpression and deletion of the amylin gene affected physical appearance with aging in rats (FIG. 2c-e). At 16 months old, ADHIP rats had dull coats, kyphosis, poor grooming and gait abnormalities, which were not seen in AD littermates. ADHIP rats developed physical deterioration and comorbidities that were not observed in AD littermates (FIG. 2k, l and Table 4). Comorbidities include glucose dysregulation and cardiac hypertrophy, which were previously^{12,17,22} reported in non-AD rats overexpressing human amylin, and sarcopenia, consistent with previous data^{23,24} showing that amylin impairs glycogen synthesis in skeletal muscle.

TABLE 2
Physical deterioration in ADHIP rats vs AD littermates.

| Comorbidities | | AD | ADHIP |
|---|---|---|---|
| Body weights | 12M (g) + SEM | 655.1 ± 18.3 | 596.8 ± 16.3* |
| (BW) | 16M (g) + SEM | 650.9 ± 19 | 472.8 ± 18.3**** |
| | BW change (%) | −0.64% | −20.8% **** |

| | | |
|---|---|---|
| Heart weight/Body weight ± | 0.0024 ± 0.000078 | 0.0037 ± 0.00031** |
| SEM | | |
| Dehydration (animal/total) | 0/17 | 5/17 |
| Lethargy (animal/total) | 0/17 | 4/17 |
| Cataracts (animal/total) | 0/17 | 4/17 |
| Abnormal gait (animal/total) | 0/17 | 3/17 |

Comorbidities include sarcopenia as measured by reduction in body weights, cardiac hypertrophy as measured by heart weight-to-body weight ratio, glucose dysregulation showed as dehydration; number of animals with lethargy, cataract formation in the eyes, and abnormal gait are included. Data are means±SEM. ADHIP vs AD: P 21 0.05 *, P<0.001 ***, P<0.0001 ****; by two-tailed, unpaired Student's t test.

Altered composition of secreted amylin in ADHIP rats was reflected in the brain magnetic resonance imaging (MRI; FIG. 2f, g) and in changes of CSF Aβ concentration (FIG. 2h) and CSF amylin-Aβ relationship with aging (FIGS. 6m, n). Brain MRI of 16 months old ADHIP rats showed an enlargement of the brain ventricles (FIG. 2f, g) that correlated with lower brain weights (FIG. 6o), compared to AD littermates. CSF Aβ concentration in ADHIP rats decreased with aging (FIG. 2h), which correlated with cerebral amylin-Aβ plaque formation (FIG. 2i). Amylin deposition was detected also in small blood vessels (FIG. 2j), especially in WM regions (FIG. 2k, FIGS. 2p and q), and in pancreatic tissue (FIG. 2l; i.e., the positive control for amylin deposition). Although rat amylin is non-amyloidogenic⁵, sparse amylin deposition was detected in brain (FIG. 2m-o) and pancreatic (FIG. 2p) tissues of AD rats. AD-AKO rats had no amylin deposition in the brain (FIG. 2q-s) or the pancreas (FIG. 2t) providing critical information that the pancreas is the source of amylin that is deposited in the brain.

Patchy areas of amylin-positive neurons were found in the brains of ADHIP rats and AD rats intravenously infused with human amylin (FIGS. 2r, 2s).

These results show that ADHIP rats mirror findings in fAD brains and that pancreatic overexpression of human amylin in AD rats accelerates aging and behavior deficits; genetic suppression of amylin expression is protective.

Example 3: Based on the MRI analysis and brain weights, amylin-associated pathology likely triggers hypoxic-ischemic brain injury.

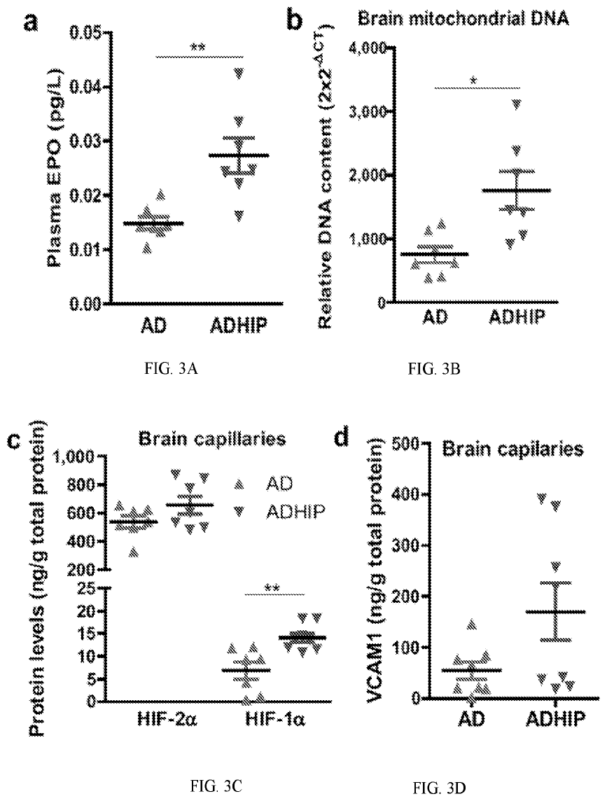

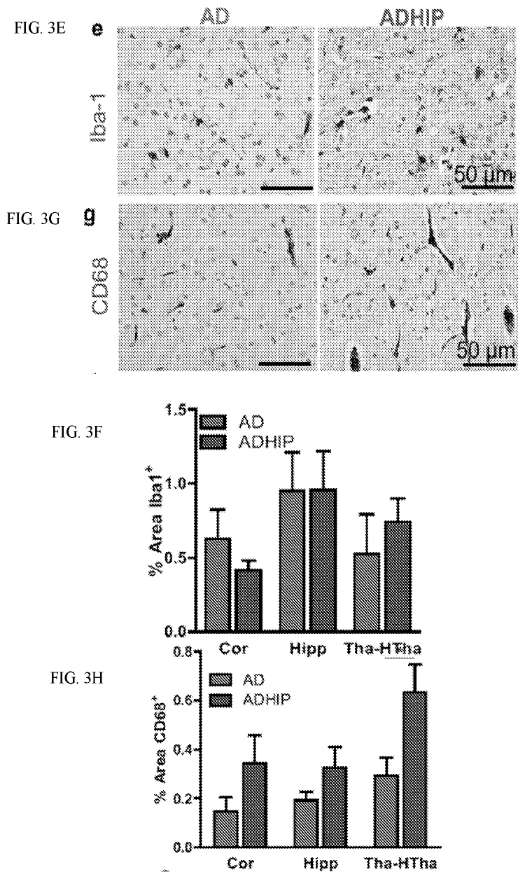

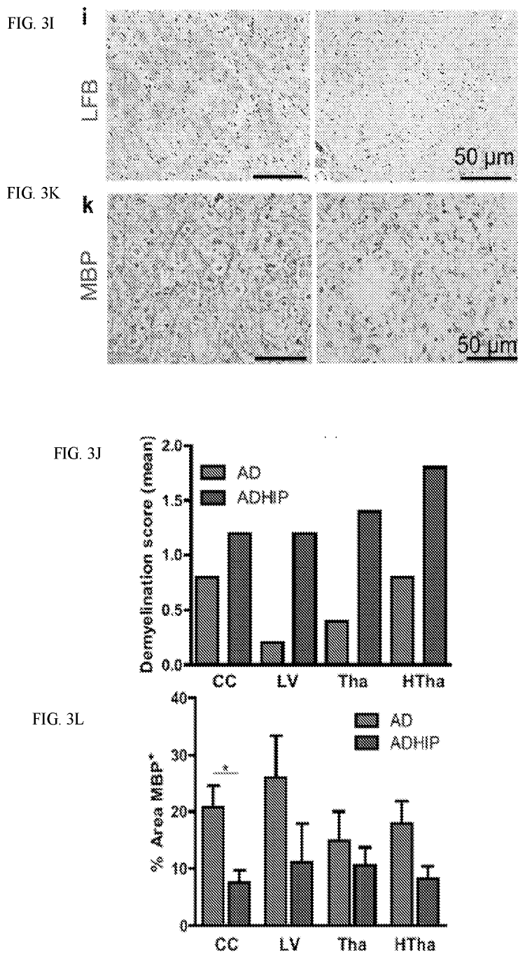

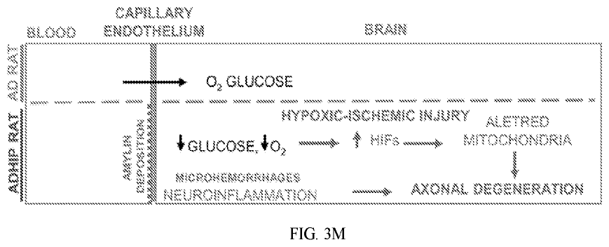

In ADHIP rats, the plasma level of erythropoietin (EPO; a marker of systemic hypoxia), the brain mitochondrial DNA content and the protein levels of hypoxia inducible factors (HIFs) and vascular cell adhesion molecule 1 (VCAM-1) in brain capillary lysates are higher than in AD rats (FIG. 3a-d and FIGS. 7a-c). Histological analysis of brain tissues showed higher number of microhemorrhages (FIGS. 7d, 7e) in ADHIP rats than in AD rats. These pathologic findings correlated with greater gliosis (FIG. 3e-h) and axonal degeneration (FIG. 3i-l) in ADHIP vs. AD rats. Activation of hypoxia signaling pathways was also found in AD rats infused intravenously with low amounts of human amylin (60 μg/kg body weight, every 3^{rd} day, for 2 months). At the end of infusion regimen, AD rats that were given human amylin accumulated amylin and hypoxia markers in the brain vasculature (FIGS. 7f-l).

Thus, peripherally-mediated amylin dyshomeostasis induced hypoxic-ischemic brain injury and axonal degeneration as a result of progressive amylin deposition in small blood vessels through mechanisms that appeared to involve upregulation of vascular adhesion proteins.

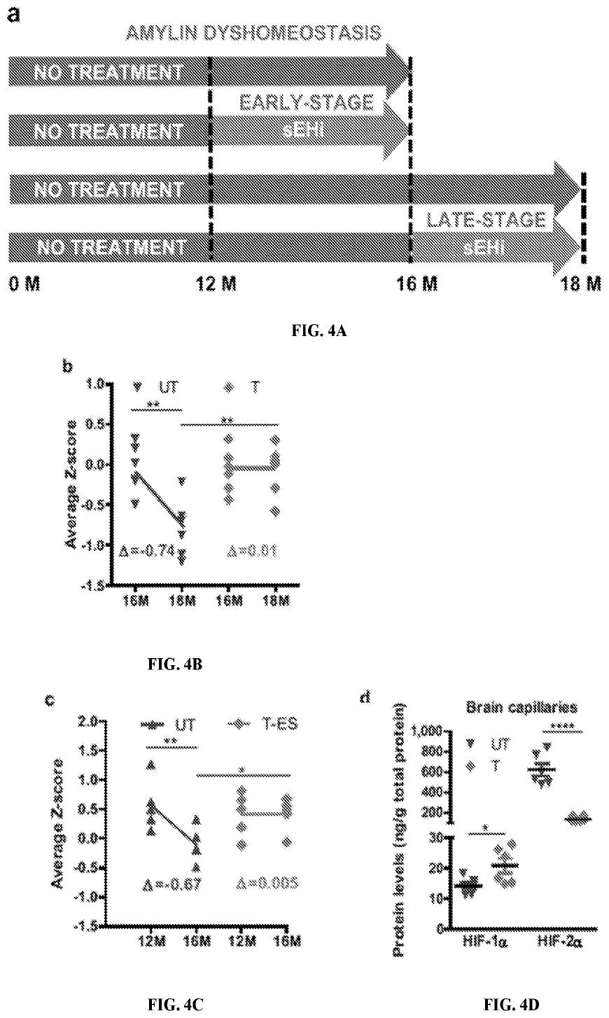

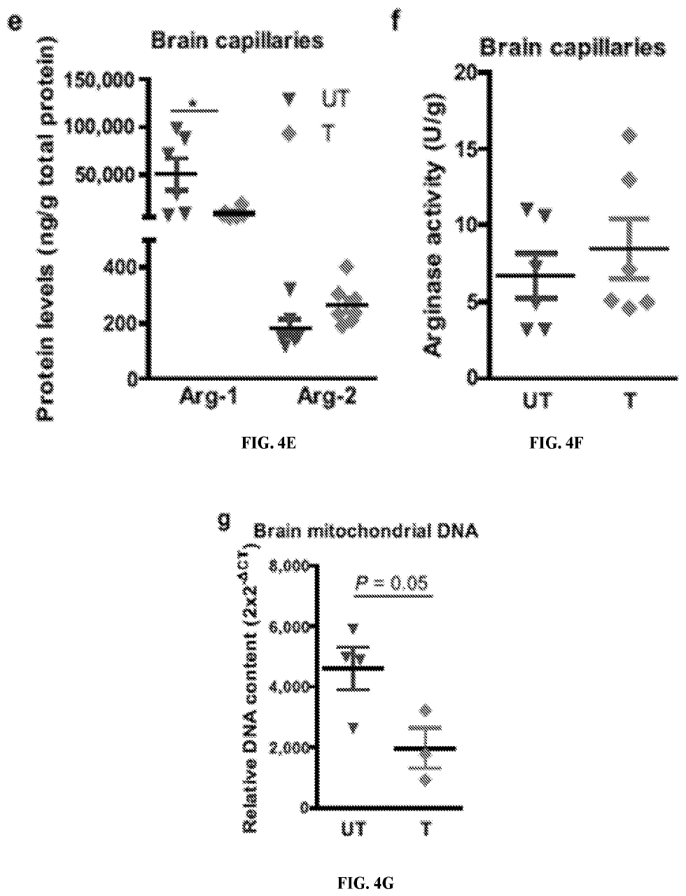

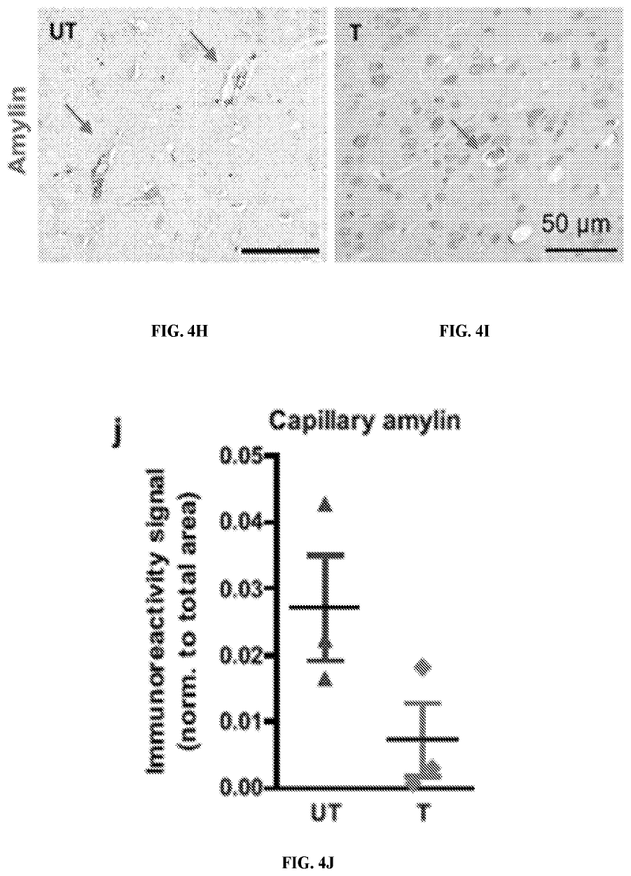

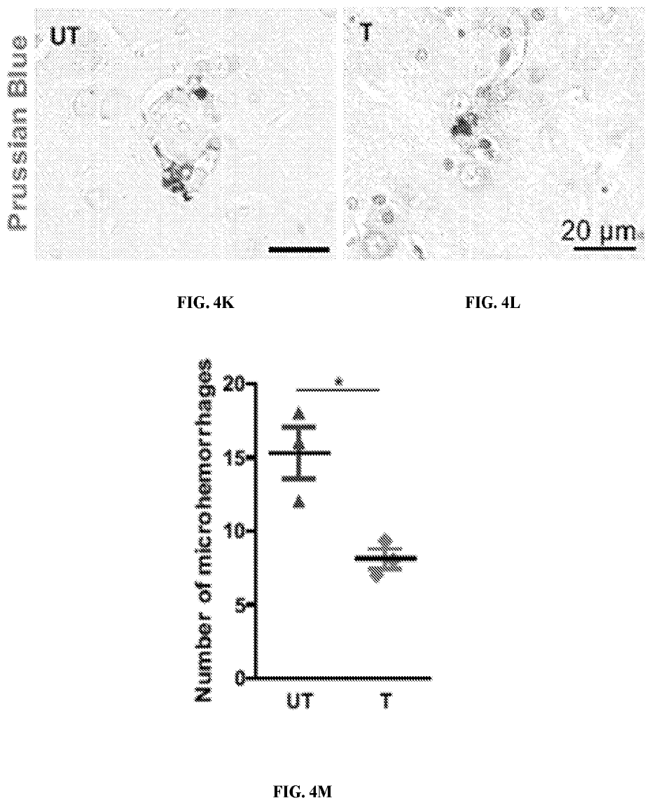

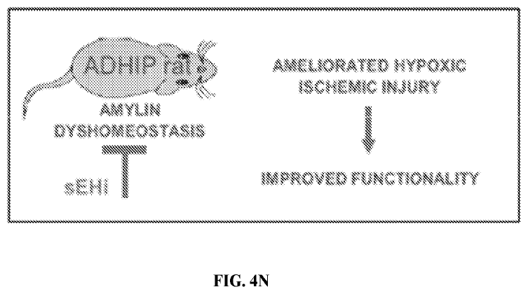

Example 4: Endothelial cell (EC)-formed epoxyeicosatrienoic acids (EETs) modulate VCAM-1 expression²³ and protected against cardiac amylin deposition in a rat model of amylin dyshomeostasis²⁴. Treatment with an inhibitor of soluble epoxide hydrolase (sEH), the enzyme that degrades EETs, reduced behavior deficits in ADHIP rats based on data from 2 separate cohorts at different disease stages, i.e., the late stage of amylin dyshomeostasis (>16 months old ADHIP rats) and the early stage of amylin dyshomeostasis (ES; 12 months old ADHIP rats) (FIG. 4a-c and FIGS. 8a-l). This treatment also lowered brain accumulation of hypoxia markers (FIG. 4d-g), which correlated with reduced amylin deposition in brain capillaries by immunohistochemical analysis (FIG. 4h-j), the number of brain microhemorrhages (FIG. 4k-m) and the extent of axonal degeneration (FIGS. 8m-p).

These data indicate that pharmacological suppression of amylin secretion reduced behavior deficits in AD rats by protecting brain capillaries from accumulation of amylin and consequent hypoxic-ischemic brain injury.

In summary, amylin dyshomeostasis modulates brain amyloid composition in human AD and that pancreatic overexpression of human amylin in AD rats accelerates pathologic aging via mechanisms that involve mixed amylin-Aβ pathology and small vessel ischemic disease (SVID); genetic or pharmacologic suppression of amylin expression is protective. Given the fact that SVID is an early pathological process in both sAD²⁵ and fAD²⁶, the data suggest that detection of amylin dyshomeostasis and therapeutic strategies to mitigate capillary accumulation of amylin could reduce SVID and cognitive decline in humans.

In sAD, central amylin dyshomeostasis is explained by aging-related insulin resistance that triggers hyperamylinemia¹⁸. Central amylin dyshomeostasis in fAD was not anticipated, given the earlier onset of disease with reduced age-dependence of amyloid pathology. The mechanisms underlying brain amylin accumulation in fAD remain unknown. The results showed no association of common and rare amylin variants with the risk of developing AD. Given the general low allelic frequency of variants in the gene, large cohorts of well characterized cases and controls will be needed to conclusively determine the role of rare variants in this gene in AD.

Overexpression of human amylin in AD and non-AD rats^{15,27} leads to an increase of blood amylin levels later in life, although rats overexpress human amylin through their entire lives. Thus a steady accumulation of amylin in tissues appears to provoke an aging-induced deficiency in protein homeostasis leading to brain amylin accumulation and behavior deficits. Future studies need to test whether the interaction of amylin dyshomeostasis with AD proteins (Aβ and tau) are specifically important to induce cognitive decline in humans.

Both overexpression and deletion of the amylin gene affect physical appearance with aging in rats. Genetic suppression of pancreatic amylin in AD rats has brought to light a potential paradoxical relationship between increased body weight and brain function. These findings suggest that amylin may play a critical role in aging, energy metabolism and brain function in ways more complex than initially considered.

### Example 5: Amylin-Mediated Regulation of LRP1 by miR-103/107 Impairs —Amyloid Efflux

### Human samples

The protocol concerning the use of autopsy tissues from patients was approved by the University of Kentucky Institutional Review Board (IRB) and informed consent was obtained prospectively. Paraffin embedded human brain tissues (n=9) provided by the Alzheimer's Disease Center biobank at the University of Kentucky was used to explore amylin-Aβ interaction in the brain microvasculature. Formalin fixed dorsolateral frontal cortex (Brodmann area 9) tissue was used from six autopsied individuals>80 years of age at death and three age-matched cognitively unaffected (CU) individuals. The disease group included patients with AD without diabetes (n=3) and AD with diabetes (n=3). The absence/presence of diabetes was determined during life (at longitudinal clinical visits) by patient or caregiver self-report and the use of diabetic medications. Neuropathological information, neuritic amyloid plaques (Consortium to Establish a Registry for Alzheimer's Disease; CERAD), Braak NFT stage and CAA severity, along with age and sex of each individual included in the present study are summarized in Table 3.

TABLE 3
Neurological Information

| | | | | | Braak | CAA | | |
|---|---|---|---|---|---|---|---|---|
| | Sex | APOE | Age | Diabetes | stage | severity | AD | CERAD |
| 1 | M | 4/4 | 93 | Yes | 5 | 2 | Yes | 3 |
| 2 | M | 3/4 | 91 | Yes | 5 | 0 | Yes | 3 |
| 3 | F | | 92 | Yes | 5 | 3 | Yes | 3 |
| 4 | M | 3/3 | 95 | No | 5 | 1 | Yes | 2 |
| 5 | F | 3/4 | 82 | No | 5 | 0 | Yes | 3 |
| 6 | F | 4/4 | 86 | No | 5 | 2 | Yes | 3 |
| 7 | F | 3/3 | 91 | No | 2 | 3 | No | 0 |
| 8 | F | 3/3 | 83 | No | 2 | 0 | No | 0 |
| 9 | M | 3/3 | 80 | No | 1 | 0 | No | 0 |

Table 3: Neuropathological information, including neuritic amyloid plaques (Consortium to Establish a Registry for Alzheimer's Disease; CERAD), Braak NFT stage and CAA severity, along with APOE genotype, absence/presence of diabetes, age and sex of each individual included in the present study.

### Experimental Animals

This investigation conforms to the Guide for the Care and Use of Laboratory Animals published by the US National Institutes of Health (NIH Publication No. 85-23, revised 1996) and was approved by the Institutional Animal Care and Use Committee at the University of Kentucky.

To study the impact of systemic pancreatic amylin dyshomeostasis on the Aβ efflux from the brain, rats were used that express human amylin in pancreatic β-cells (i.e., HIP rats)³⁶. The HIP rats (non-AD rats) develop systemic amylin dyshomeostasis by ˜10-12 months of age, which is characterized by amylin deposition in the pancreas³⁶ and extra-pancreatic tissues ^{18,25-28,} including the brain microvasculature¹⁸. Breeding pairs were purchased from Charles River Laboratory. Wild type (WT) littermates expressing non-amyloidogenic rat amylin served as controls.

In vitro BBB model of Aβ₄₂ transcytosis with amylin-induced stress at the blood side. An in vitro BBB model described previously³⁷ was used with modifications related to amylin deposition on the EC monolayer and consequent effects on the Aβ transport across the EC monolayer. Briefly, primary rat brain microvascular endothelial cells (Cell Applications Inc) were plated on 24-well Transwell-Clear inserts with 0.4 μm pore polycarbonate membrane (Costar, Corning, N.Y., USA) and primary rat brain astrocytes (Sigma) were cultured at the bottom wells. Barrier integrity was measured from the trans-endothelial electrical resistance (TEER) as described previously^{37,38} using the EVOM2 meter with STX-3 electrodes (World Precision Instruments). Maximum TEER was achieved within 8-10 days in culture. BBB permeabilities to human amylin (10 μM; Anaspec; AS-60254-1), rat amylin (10 μM; American Peptide) and DMSO (1 mM; vehicle) were assessed using FITC-Dextran 4 kDa (Fisher Scientific) diluted in Hank's Balanced Salt Solution (HBSS) buffer with 0.1% BSA (HBSS-BSA) as a paracellular diffusion marker. Permeability coefficients were calculated using the formula; P=(ΔQ/Δt)/(A*C₀), (ΔQ/Δt)=rate of FITC-Dextran change; A=surface area of insert (0.33cm²); C₀=Initial FITC-Dextran input.

In the Aβ₄₂ transcytosis experiments, the EC monolayer was treated with a medium containing human amylin (10 μM) or vehicle (DMSO) for 24 hours. After washing, the luminal chamber was replaced with HBSS-BSA, and the abluminal chamber with Aβ₍₁₋₄₂₎-FAM (5 μM; Bachem) or FITC-Dextran, respectively. Aβ₍₁₋₄₂₎ samples were collected from the luminal chamber for the measurement of the Aβ₍₁₋₄₂₎-FAM and FITC-Dextran fluorescence intensities and Aβ₍₁₋₄₂₎ transcytosis quotient (TQ) as described previously³⁸: TQ=(Aβ₍₁₋₄₂₎−FAMₗᵤₘᵢₙₐₗ/Aβ₍₁₋₄₂₎−FAMᵢₙₚᵤₜ)/(FITC-Dextranₗᵤₘᵢₙₐₗ−FITC-DEXTRANᵢₙₚᵤₜ).

### MicroRNA Mimics and Antagomir Transfection

To study the role of miRNA signaling in amylin-induced suppression of endothelial LRP1 expression, transfection of rat brain microvascular ECs was used with miR-103-3p (MCR01039)agcagcauuguacagggcuauga(SEQ ID NO: 5), miR-107-3p (MCR01045)agcuucuuuacaguguugccuugu(SEQ ID NO: 6) and control (MCH00000) (https://www.abmgood.com/mirna-mimic-negative-control-mch00000.html). Antagomir miR-103-3p (IH-320345-05-0005)(SEQ ID NO: 5), miR-107-3p (IH-320348-05-0005)(SEQ ID NO: 6) and negative control (IN-001005-01-05) (https://www.biocompare.com/22445-RNA/4995709-miRIDIAN-microRNA-Hairpin-Inhibitor-Negative-Control-1-5-nmol/#productspecs) (Dharmacon Inc.) were used in an attempt to rescue LRP1 expression. All transfections were done using RNAiMAX (Invitrogen) as per manufacturer's recommended protocol. Briefly, ECs were plated at 50% confluency in 6-well plates followed by co-transfection with either 100 nM of 103-3p and 107-3p mimics or antagomirs along with their respective negative controls. After 12-hours, antagomir-treated cell groups were further treated with 10 uM human amylin for 24-hours. After 36-hours of transfection, cells were harvested for Western blot analysis.

### Real-Time Quantitative Reverse Transcription PCR

Total RNA was isolated using RNAqueous total RNA isolation kit according to manufacturer's protocol (Invitrogen, AM1914). cDNA synthesis and amplification were done using iTaq Universal SYBR Green One-Step Kit (Biorad; 1725151) with the following primer sequences: LRP1: forward (Fwd) 5′-TTGTGCTGAGCCAAGACATC-3′(SEQ ID NO: 7), reverse (Rev) 5′-GGCGTGGAAGACATGTAGGT-3′(SEQ ID NO: 8); and GAPDH: Fwd 5′- GCTGCGTTTTACACCCTTTC-3′(SEQ ID NO: 9), Rev 5′-GTTTGCTCCAACCAACTGC-3′(SEQ ID NO: 10) (IDT, Inc, USA). For miRNA quantification, cDNA was synthesized from total RNA using miRNA cDNA synthesis kit with poly (A) polymerase (ABMgood, G902). cDNA was amplified using SYBR Green mastermix (Biorad) along with miRNA specific primers from (rno-miR-103-3p, MPR00332; rno-miR-107-3p, MPRO0335; RNU6 house Keeping gene, MP-r99998) (ABMgood). Data were analyzed using the 2^{-ΔΔCt} method, and experiments were normalized to GAPDH or U6 miRNA

### MTS Cytotoxicity Assay

CellTiter 96® AQueous One Solution Cell Proliferation Assay (MTS) (Promega) was used to assess cytotoxicity of aggregated amylin on the EC monolayer.

### Immunohistochemistry, Immunofluorescence and Immunocytochemistry

For immunohistochemistry, formalin-fixed, paraffin-embedded brain tissues from humans and rats were processed as described before^{16,18,28,39}. Antibodies against amylin (1:200; clone E5; SC-377530; Santa Cruz) and Aβ (1:300; CST2454; Cell Signaling Technology) were the primary antibodies. Biotinylated IMPRESS horse anti-rabbit-AP conjugated (2 drops/slide; MP-5401; Vector), biotinylated horse-anti mouse (1:300, BA-2000, Vector) were secondary antibodies. The specificity of the amylin antibody in both human and rat brain tissues was established in previous studies^{16,18,28,39}. Pancreatic tissue from rats with deleted amylin gene (AKO rats) was the negative control for amylin. The generation of AKO rat model was described previously¹⁸.

In immunofluorescence experiments, formalin-fixed, paraffin-embedded human brain tissue was used and processed as previously described^{16,18,39}, brain capillaries from HIP and WT rats isolated as previously described¹⁸. and cultured ECs. Anti-amylin (1:200; clone E5; SC-377530; Santa Cruz), anti-collagen IV (1:500; ab6586; abcam), anti-alpha smooth muscle actin-Alexa Fluor 405 (1:200; ab210128, abcam), anti-caveolin-1 (1:100; sc-894; Santa Cruz, Tex.), anti-LRP1 (1:500; sc-57351; Santa Cruz), anti-4HNE (1:200; ab46545; abcam) were the primary antibodies. Secondary antibodies were: Alexa Fluor 488 conjugated anti-mouse IgG (1:300; A11029; Invitrogen), Alexa Fluor 568 conjugated anti-rabbit IgG (1:200; A11036; Invitrogen), Alexa Fluor 568 conjugated anti-mouse IgG (1:300; A11004; Invitrogen). Nuclei were counterstained with DAPI mounting media. For triple staining of human brain tissues, smooth muscle actin-Alexa Fluor 405 antibody was added after staining with human amylin and collagen IV; DAPI free mounting media was used. For Thioflavin S staining, after secondary antibody incubation, brain slides were incubated in 0.5% Thioflavin S for 30 minutes at room temperature. Slides were then incubated for 3 minutes in 70% ethanol, 5 minutes in 0.2% Sudan black before washing and mounting. Immunocytochemistry was performed as described previously (39, 40).

### Immunoprecipitation

To immunoprecipitate rat Aβ from brain homogenates and plasma, a previously published protocol (16) was used. Briefly, 1000 μg of protein was incubated with anti-rat and human Aβ (2 μg; CST2454; Cell Signaling Technology) overnight with end-over-end rotation, at 4° C. All of the elution was used for Western blot analysis.

### Western Blot and Enzyme-Linked Immunosorbent Assay (ELISA)

Western blot analysis was performed on isolated brain capillaries, brain tissue homogenate and plasma from rats. Tissues were processed as described previously^{16,18,28,39}. RIPA buffer with 2% SDS was used to retrieve Aβ monomers from frozen brain samples⁴¹. The lysate was centrifuged at 17,000×G for 30-minnutes. The supernatant was separated from pellet after centrifugation and was then used for Western blotting. Total protein levels were estimated using a BCA kit (23225, ThermoFisher). Anti-LRP1 antibody recognizing the β-subunit of LRP1 (1:1,000; clone 5A6; sc-57351; Santa Cruz), rabbit anti-amylin polyclonal (1:2,000; T-4157, Bachem-Peninsula Laboratories, CA), anti-rat and human Aβ (1:1,000; 2454; Cell Signaling Technology), mouse anti-β actin (1:10,000; clone BA3R; MA5-15739; ThermoFisher), mouse monoclonal anti-GAPDH (1:10,000; clone 6C5; ab8245; Abcam), anti-rabbit IgG HRP conjugated (1:30,000; NA934VS; GE Healthcare) and anti-mouse IgG HRP conjugated (1:20,000; NXA931; GE Healthcare) were primary antibodies. Immunoprecipitated rat Aβ from brain homogenates and matched plasma (50 μg of protein from tissue homogenate or immunoprecipitated rat Aβ elution) were loaded on 8% SDS-PAGE gel. Aggregated Aβ from brain homogenates were resolved in native-PAGE (non-reducing; non-denatured). Monomeric Aβ peptides were resolved in acidic Bis-Tris gel with 8M urea³⁵. To enhance signal for monomeric Aβ, membranes were boiled for 3 minutes in PBS before the blocking step. LRP1 in cell and brain capillary lysates was resolved using 4-12% Bis-Tris gel under non-reducing condition. HRP-conjugated anti-rabbit or anti-mouse were secondary antibodies. Equal loading in Western blot experiments was verified by re-probing with a monoclonal anti-β actin antibody (raised in mouse, clone BA3R, Thermo Scientific; 1:2000). Protein levels were compared by densitometric analysis using ImageJ software.

Levels of amylin in the rat plasma and red blood cells were measured using amylin ELISA kits (EZHA-52K, Millipore), according to the manufacturer's protocol.

### Lipid Peroxidation and Reactive Oxygen Species (ROS)

Lipid peroxidation and ROS were measured in cultured rat brain microvascular ECs using previously published protocols^{26,39}.

### Statistical Analysis

Parametric comparison of two groups was done using two-tailed unpaired t-test. Welch's correction was used with t-test to account for unequal variance from unequal sample sizes, if necessary. Parametric comparisons between three groups or more were performed using one-way or two-way ANOVA with Dunnett's post hoc or Tukey's post hoc tests. Data are presented as mean±S.E.M. Difference between groups was considered significant when P<0.05. All analyses were performed using GraphPad Prism 8.1 software.

### Results

1. Aβ deposition in perivascular spaces co-occurs with amylin accumulation in vessel wall.

Co-staining of human brain sections with anti-amylin and anti-Aβ antibodies identified amylin immunoreactivity (brown) on the luminal side of small arterioles that co-occurred frequently with patchy areas of Aβ immunoreactivity (green) within Virchow-Robin spaces, in AD but not CU individuals (FIG. 9A-9C). Confocal microscopy analysis of AD brain sections confirmed amylin deposition (green) on the luminal side and Aβ deposits (red) in perivascular spaces (FIG. 9D). The analysis also indicated amylin-Aβ tangled within the vessel wall (FIG. 9E). To confirm amylin accumulation within the vessel wall, AD brain sections for amylin was triple-stained, collagen and smooth muscle actin. Confocal microscopy analysis (FIGS. 9F and 9G) shows a propensity for amylin deposition at the vascular luminal side.

The results of the instant exploratory study in human brains reveal histological evidence of interaction between amylin secreted from the pancreas and Aβ at the blood-brain interface, in AD. Tangled amylin-Aβ deposits across cerebral blood vessel walls were identified in AD brains from individuals with AD independent of comorbid type-2 diabetes, consistent with previous studies¹⁶. The presence of amylin deposition at the luminal side of small blood vessels and Aβ in perivascular spaces suggest that systemic amylin dyshomeostasis may contribute to impaired Aβ efflux from the brain into the bloodstream in individuals with AD.

2. Tangled amylin-Aβ across the BBB impairs the Aβ efflux from the brain, in rats.

To study in vivo how systemic pancreatic amylin dyshomeostasis impairs Aβ transcytosis across the BBB, HIP rats that express amyloid-forming human amylin in pancreatic β-cells³⁶ and accumulate amylin in brain capillaries¹⁸ were used. The average circulating level of amylin in 16-month old HIP rats was ˜2-fold higher compared to that in wild type (WT) littermates (FIG. 15A). This was associated with amylin accumulation in small cerebral arterioles (FIG. 15B) and presumable capillaries (FIG. 15C; arrows), which was not detected in WT rat brains (FIG. 15D).

Co-staining of brain slices from HIP and WT rat brains with anti-Aβ (green) and anti-amylin (brown) antibodies showed vascular amylin-Aβ interaction in HIP, but not WT rats (FIG. 10A-E). Pancreatic tissue from HIP (FIG. 15E) and amylin knockout (AKO) (FIG. 15F) rats served as positive and negative controls, respectively. Brain slices from age-matched APP/PS1 rats were the positive control for Aβ deposits (FIG. 16A). In HIP rat brains, amylin immunoreactivity was detected solely on the luminal side of the blood vessel, whereas those of Aβ were seen within perivascular, Virchow-Robin spaces (FIG. 10A) and within the blood vessel wall (FIG. 10B). In addition, brain slices from HIP rats had sporadic amylin-Aβ deposits (FIG. 10C; circle) that were seen in association with capillaries positive for luminal amylin accumulation and for Aβ deposition within the surrounding tissue (FIG. 10C; arrows), consistent with the findings in human AD brains (FIG. 9A-C). Scattered Aβ immunoreactivity was also detected in HIP rat brains (FIG. 16B), but not in brains of WT littermates (FIG. 16C).

Western blot analysis of HIP rat brain homogenates shows accumulation of Aβ in the brain (FIG. 10F and FIG. 16D). Rat Aβ₄₀ peptide and brain homogenate from a 12-month old APP/PS1 rat were the positive controls for Aβ accumulation. To test whether both soluble and insoluble aggregated Aβ accumulated in HIP rat brains, native-PAGE was performed followed by Western blot (FIG. 10G). The levels of soluble and insoluble Aβ aggregates were higher in HIP rat brains compared to those in WT littermates.

AD model rats are genetically determined to develop brain Aβ pathology, whereas rats expressing human amylin in the pancreatic islets may accumulate Aβ in the brain due to changes associated with chronically elevated blood levels of human amylin. To test whether the Aβ efflux from the brain to bloodstream is altered in HIP vs. WT rats, immunoprecipitation was used to enrich Aβ in plasma samples and brain homogenates from age-matched rats in the two groups followed by Western blot analysis of Aβ (FIGS. 11A and 11B). The ratio of plasma-to-brain Aβ levels was lower in HIP compared to WT rats (FIG. 11C), which suggests that Aβ efflux across the BBB is impaired in HIP rats.

Taken together, the results show that increased pancreatic secretion of amyloid-forming amylin is associated with: 1, amylin accumulation in brain capillaries (FIGS. 10A-10C, FIG. 15B and 15C); 2, tangled amylin-Aβ deposits across the blood vessel wall (FIGS. 10A and 10B); 3, Aβ accumulation in the brain (FIGS. 10F and 10G); and 4, impaired Aβ efflux across the BBB (FIGS. 11A-11C).

3. High blood human amylin suppresses the Aβ efflux transporter expression.

Amylin deposition in the brain microvasculature may induce stress in ECs and decline of the Aβ efflux transporter LRP1 expression. To test this hypothesis, LRP1 protein expression was analyzed in brain capillary lysates from aged HIP rats vs. WT littermates and EC lysates from EC monolayers that were subjected to amylin-induced stress.

Vascular amylin-induced LRP1 downregulation in the brain endothelium. Brain capillaries were isolated from HIP and WT rats and tested for the presence of amylin deposition and LRP1 protein expression by immunofluorescence and Western blot. Confocal microscopy analysis of isolated brain capillaries (FIG. 11D) showed that amylin deposition (green) co-localized with caveolin-1 (red), a protein that is abundant in ECs and further confirmed amylin deposition in HIP brain capillaries (FIG. 11E). Staining for LRP1 revealed lower LRP1 immunoreactivity signal in brain capillaries from HIP rats compared to WT littermates (FIG. 11F-G). Consistent with this result, Western blot analysis showed reduced LRP1 protein levels in brain capillary lysates from HIP rats compared to those in WT littermates (FIG. 11H and 11I). In contrast to protein expression, LRP1 mRNA levels were increased in HIP vs. WT rat brain capillaries (FIG. 11J), suggesting that the decrease in LRP1 protein in HIP rat capillary ECs occurs at a post-transcriptional level.

Amylin-induced LRP1 downregulation in endothelial cells, in vitro. To further evaluate the relationship between vascular amylin deposition and LRP1 protein expression, rat brain microvascular ECs was incubated with various concentrations of human amylin for 24 hours followed by analysis of LRP1 protein expression by Western blot (FIG. 12A). LRP1 protein levels decreased with increasing concentrations of human amylin; LRP1 expression was reduced by more than 50% in ECs incubated with 10 μM human amylin. This result was further confirmed by immunofluorescence measurements in ECs incubated for 24-hours with 10 μM human amylin (FIG. 12B). In contrast, non-amyloidogenic rat amylin (10 μM; 24-hour incubation time) had no effect on LRP1 protein levels (FIG. 12B-D), as indicated by analyses of immunoreactivity by confocal microscopy (FIG. 12B) and Western blot (FIG. 12C). Viability of the ECs was not affected by incubation with human amylin (FIG. 17), indicating that decreased LRP1 protein expression is not due to cell death. Consistent with this result, the capacity of ECs to induce transcript expression of LRP1 was not affected by amylin stress; consistent with the findings in HIP rat cerebral capillaries, LRP1 mRNA levels were greatly elevated in ECs incubated with human amylin vs. control cells and ECs incubated with rat amylin (FIG. 12D).

Impaired endothelial Aβ transcytosis by amylin in a 3-dimensional BBB model. To determine whether the amylin stress-induced LRP1 downregulation affects Aβ transcytosis, a well-established model of BBB³⁷ was employed in which the EC monolayer was exposed to human amylin on the luminal side (as shown in FIG. 12E). The BBB model was tested for monolayer formation and tightness by measuring TEER (FIG. 12F). All experiments were done with a fully formed EC monolayer characterized by the maximum TEER=110±5 Ω/cm².

First, the effect of human amylin was tested on EC monolayer structural integrity. Aβ transcytosis was measured across the BBB using FAM tagged Aβ₄₂ (Aβ₄₂-FAM) and FITC-dextran as a paracellular diffusion marker. TEER, cell morphology within the EC monolayer and permeability to FITC-dextran (4 kDa) were measured following the incubation of the ECs for 24-hours with 10 μM human amylin or similar concentrations of rat amylin or vehicle (FIG. 18A-18E). Although TEER decreased following the treatment with human amylin (FIG. 18B), neither the intercellular space, as assessed from FITC-dextran permeability (FIG. S4C-D), nor EC monolayer morphology (FIG. 18E) were significantly altered by the treatment with human amylin. To assess the impact of amyloid-forming amylin on Aβ transcytosis across the BBB, human amylin or vehicle were applied at the luminal (blood) side for 24-hours followed by washing of the EC monolayers with PBS and application of FITC-Dextran or Aβ₄₂-FAM at abluminal (brain) side of the BBB for 1-hour. The amounts of Aβ₄₂-FAM and FITC-Dextran that cross the monolayer were estimated from the fluorescence intensity in the medium samples collected from luminal side and used to calculate the Aβ transcytosis quotient³⁸. Amylin-pretreatment reduced the Aβ transcytosis quotient by 20+5% (P<0.05) (FIG. 12G), which indicates that amylin blocks transcytosis of Aβ₄₂ across the in vitro BBB.

From these results (FIG. 12), amylin accumulation at the luminal side of the BBB impairs Aβ transcytosis across the ECs via LRP1 downregulation. As the LRP1 transcript was upregulated in response to amylin effects on ECs both in vivo (FIG. 11J) and ex vivo (FIG. 12D), this suggests that LRP1 protein downregulation may involve altered protein translation.

4. Aβ efflux transporter expression is suppressed by amylin-induced endothelial cell stress.

Paralog miRNAs miR-103 and miR-107 are upregulated by oxidative stress⁴² and repress LRP1 translation in several cell lines³². Thus, to determine if these miRNAs are involved in amylin-induced LRP1 downregulation in the BBB.

Amylin accumulation in brain capillaries induced oxidative stress in ECs by forming deposits with biochemical properties of amyloid (FIG. 13A), which was shown to alter structural stability of the cellular membranes^{25,26,39}. This is evidenced by accumulation of the lipid peroxidation marker 4-hydroxynonenal (4-HNE) (FIG. 13B). Oxidative stress also occurred in rat brain microvascular ECs incubated with human amylin (10 μM amylin; 24-hour incubation time), as indicated by lipid peroxidation of the EC membranes (FIG. 13C-13E) and increased generation of ROS (FIGS. 13F and 13G). Pancreas from a HIP rat was the positive control (FIG. 19A) and pancreas tissue from an AKO rat was the negative control (FIG. 19B) for amylin amyloid. The amylin stress on ECs was associated with elevated levels of miR-103 and miR-107 (FIG. 13H). Brain capillary lysates from HIP rats also had elevated miR-103 and miR-107 levels compared to those in WT littermates (FIG. 13I).

Next, ECs were pre-treated with poloxamer 188, a surfactant that decreases lipid peroxidation in cellular membranes^{26,39}. Surfactant molecules blocked lipid peroxidation and consequent ROS production (FIG. 13C-G; magenta bars); however, the surfactant neither normalized miR-103/107 levels (FIG. 13H) nor rescued LRP1 expression (FIG. 13J) upon amylin-induced EC damage.

The results support the hypothesis that miRNA is upregulatied by amylin-mediated endothelial stress and suggest that additional pathways may compromise the capability of ECs to express LRP1, which were not linked to peroxidative membrane injury.

5. Antisense microRNAs rescue amylin-induced suppression of Aβ efflux transporter.

TargetScan predicts that miR-103 and miR-107 bind directly to LRP1, with the biding site located at the 3′UTR region of rat LRP1 (FIG. 14A). To further determine whether high miR-103 and miR-107 levels suppress LRP1 translation, miR-103 and miR-107 mimics (100 nM) were co-transfected into rat brain microvascular ECs. Cell lysates were tested after 24-hours for LRP1 protein expression by Western blot. The average LRP1 expression level was lower in ECs co-transfected with miR-103 and miR-107 mimics compared to miR-control (FIG. 14B).

Antisense microRNAs are used to target aberrant miRNA⁴³. Antagomir (amiR) 103 and amiR-107 was used to test the hypothesis that silencing amylin-induced upregulation of miR-103 and miR-107 rescues LRP1 expression. The instant results show that amiR-103/107 rescued LRP1 expression in ECs following amylin-induced cell stress (FIG. 14C).

These results indicate that: 1, endothelial LRP1 downregulation associated with amylin stress is a miRNA-based translational repression mechanism; and 2, LRP1 downregulation by amylin-induced stress on ECs can be reversed by modulating miR-103 and miR-107.

It will be understood that various details of the presently disclosed subject matter can be changed without departing from the scope of the subject matter disclosed herein. Furthermore, the foregoing description is for the purpose of illustration only, and not for the purpose of limitation.

All publications, patents, and patent applications mentioned in this specification are herein incorporated by reference to the same extent as if each individual publication, patent, or patent application was specifically and individually indicated to be incorporated by reference, including the references set forth in the following list:

### REFERENCES

1. Selkoe D J (1991) The molecular pathology of Alzheimer's disease. Neuron 6: 487-498.
2. Hardy J, Allsop D (1991) Amyloid deposition as the central event in the aetiology of Alzheimer's disease. Trends in Pharmac 12: 383-388.
3. Beyreuther K, Masters C L (1991) Amyloid precursor protein (APP) and beta A4 amyloid in the etiology of Alzheimer's disease: precursor-product relationships in the derangement of neuronal function. Brain Pathol 1: 241-251
4. Selkoe D J, Hardy J. The amyloid hypothesis of Alzheimer's disease at 25 years. EMBO Mol Med. 2016 Jun. 1; 8(6):595-608. doi: 10.15252/emmm.201606210
5. Westermark, P., Engstrom, U., Johnson, K. H., Westermark, G. T. & Betsholtz, C. Islet amyloid polypeptide: pinpointing amino acid residues linked to amyloid fibril formation. Proc Natl Acad Sci USA 87, 5036-5040, doi:10.1073/pnas.87.13.5036 (1990).
6. Lutz, T. A. Control of energy homeostasis by amylin. Cell Mol Life Sci 69, 1947-1965, doi:10.1007/s00018-011-0905-1 (2012).
7. Westermark, P., Andersson, A. & Westermark, G. T. Islet amyloid polypeptide, islet amyloid, and diabetes mellitus. Physiological Reviews 91, 795-826, doi:10.1152/physrev.00042.2009 (2011).
8. Maianti J P, McFedries A, Foda Z H, Kleiner R E, Du X Q, Leissring M A, Tang W J, Charron M J, Seeliger M A, Saghatelian A, Liu D R. Anti-diabetic activity of insulin-degrading enzyme inhibitors mediated by multiple hormones. Nature. 2014; 511:94-8
9. Jurgens, C. A. et al. Beta-cell loss and beta-cell apoptosis in human type 2 diabetes are related to islet amyloid deposition. Am J Pathol 178, 2632-2640, doi:10.1016/j.ajpath.2011.02.036 (2011).
10. Despa, S. et al. Hyperamylinemia contributes to cardiac dysfunction in obesity and diabetes: a study in humans and rats. Circulation research 110, 598-608, doi:10.1161/CIRCRESAHA.111.258285 (2012).
11. Jackson, K. et al. Amylin deposition in the brain: A second amyloid in Alzheimer disease? Annals of Neurology 74, 517-526, doi:10.1002/ana.23956 (2013).
12. Oskarsson, M. E. et al. In vivo seeding and cross-seeding of localized amyloidosis: a molecular link between type 2 diabetes and Alzheimer disease. Am J Pathol 185, 834-846, doi:10.1016/j.ajpath.2014.11.016 (2015).
13. Fawver, J. N. et al. Islet amyloid polypeptide (IAPP): a second amyloid in Alzheimer's disease. Curr Alzheimer Res 11, 928-940 (2014).
14. Verma, N. et al. Intraneuronal Amylin Deposition, Peroxidative Membrane Injury and Increased IL-1beta Synthesis in Brains of Alzheimer's Disease Patients with Type-2 Diabetes and in Diabetic HIP Rats. J Alzheimers Dis 53, 259-272, doi:10.3233/JAD-160047 (2016).
15. Ly, H. et al. Brain microvascular injury and white matter disease provoked by diabetes-associated hyperamylinemia. Ann Neurol 82, 208-222, doi:10.1002/ana.24992 (2017).
16. Schultz, N., Byman, E., Fex, M. & Wennstrom, M. Amylin alters human brain pericyte viability and NG2 expression. Journal of Cerebral Blood Flow & Metabolism 37, 1470-1482, doi:10.1177/0271678X16657093 (2016).
17. Schultz, N., Byman, E., Bank, T. & Wennstrom, M. Levels of retinal IAPP are altered in Alzheimer's disease patients and correlate with vascular changes and hippocampal IAPP levels. Neurobiology of Aging 69, 94-101, doi: 10.1016/j .neurobiolaging.2018.05.003 (2018).
18. Biessels, G. & Despa, F. Cognitive decline and dementia in diabetes mellitus: mechanisms and clinical implications. Nature Reviews Endocrinology 14, 1-14, doi:10.1038/s41574-018-0048-7 (2018).
19. Bertens D, Tijms B M, Scheltens P, Teunissen C E, Visser P J. Unbiased estimates of cerebrospinal fluid β-amyloid 1-42 cutoffs in a large memory clinic population. Alzheimers Res Ther. 2017 Feb. 14; 9(1):8. doi: 10.1186/s13195-016-0233-7
20. Matveyenko, A. V. & Butler, P. C. Islet amyloid polypeptide (IAPP) transgenic rodents as models for type 2 diabetes. ILAR journal 47, 225-233, doi:10.1093/ilar.47.3.225 (2006).
21. Cooper, G. J. et al. Amylin found in amyloid deposits in human type 2 diabetes mellitus may be a hormone that regulates glycogen metabolism in skeletal muscle. Proc Natl Acad Sci USA 85, 7763-7766 (1988).
22. Leighton, B. & Cooper, G. J. Pancreatic amylin and calcitonin gene-related peptide cause resistance to insulin in skeletal muscle in vitro. Nature 335, 632-635, doi:10.1038/335632a0 (1988).
23. Node K, Huo Y, Ruan X, et al. Anti-inflammatory properties of cytochrome P450 epoxygenase-derived eicosanoids. Science. 1999; 285:1276-1279.
24. Despa, S. et al. Cardioprotection by controlling hyperamylinemia in a “humanized” diabetic rat model. Journal of the American Heart Association 3, doi:10.1161/JAHA.114.001015 (2014).
25. Lindemer, E. R. et al. White matter signal abnormality quality differentiates mild cognitive impairment that converts to Alzheimer's disease from nonconverters. Neurobiol Aging 36, 2447-2457, doi:10.1016/j.neurobiolaging.2015.05.011 (2015).
26. Lee, S. et al. White matter hyperintensities are a core feature of Alzheimer's disease: Evidence from the dominantly inherited Alzheimer network. Ann Neurol 79, 929-939, doi:10.1002/ana.24647 (2016).
27. Srodulski, S. et al. Neuroinflammation and neurologic deficits in diabetes linked to brain accumulation of amylin. Molecular Neurodegeneration 9, 30, doi:10.1186/1750-1326-9-30 (2014).
28. Cohen, R. M. et al. A Transgenic Alzheimer Rat with Plaques, Tau Pathology, Behavioral Impairment, Oligomeric Aβ, and Frank Neuronal Loss. The Journal of Neuroscience 33, 6245-6256, doi:10.1523/JNEUROSCI.3672-12.2013 (2013).
29. Matveyenko, A. V. & Butler, P. C. Islet amyloid polypeptide (IAPP) transgenic rodents as models for type 2 diabetes. ILAR journal 47, 225-233, doi:10.1093/ilar.47.3.225 (2006).
30. Nirogi, R. et al. A simple and rapid method to collect the cerebrospinal fluid of rats and its application for the assessment of drug penetration into the central nervous system. J Neurosci Methods 178, 116-119, doi:10.1016/j.jneumeth.2008.12.001 (2009).
31. Song, M. K., Lin, F. C., Ward, S. E. & Fine, J. P. Composite variables: when and how. Nurs Res 62, 45-49, doi:10.1097/NNR.0b013e3182741948 (2013).
32. Dimmock, D., Tang, L. Y., Schmitt, E. S. & Wong, L. J. Quantitative evaluation of the mitochondrial DNA depletion syndrome. Clin Chem 56, 1119-1127, doi:10.1373/clinchem.2009.141549 (2010).
33. Gladman J T, Corriveau R A, Debette S, et al. Vascular contributions to cognitive impairment and dementia: Research consortia that focus on etiology and treatable targets to lessen the burden of dementia worldwide. Alzheimer's Dement. Transl. Res. Clin. Interv. 2019; 5:789-796.
34. Hainsworth A H, Minett T, Andoh J, et al. Neuropathology of white matter lesions, blood-brain barrier dysfunction, and dementia. Stroke. 2017;48:2799-2804.
35. Lindemer E R, Salat D H, Smith E E, et al. White matter signal abnormality quality differentiates mild cognitive impairment that converts to Alzheimer's disease from nonconverters. Neurobiol. Aging. 2015; 36:2447-2457.
36. Lee S, Viqar F, Zimmerman M E, et al. White matter hyperintensities are a core feature of Alzheimer's disease: Evidence from the dominantly inherited Alzheimer network. Ann. Neurol. 2016; 79:929-939.
37. Wardlaw J M, Smith C, Dichgans M. Small vessel disease: mechanisms and clinical implications. Lancet Neurol. 2019; 18:684-696.
38. Barrett E J, Liu Z, Khamaisi M, et al. Diabetic microvascular disease: An endocrine society scientific statement. J. Clin. Endocrinol. Metab. 2017; 102:4343-4410.
39. Sims-Robinson C, Kim B, Rosko A, Feldman E L. How does diabetes accelerate Alzheimer disease pathology? Nat. Rev. Neurol. 2010; 6:551-559.
40. Arnold S E, Arvanitakis Z, Macauley-Rambach S L, et al. Brain insulin resistance in type 2 diabetes and Alzheimer disease: Concepts and conundrums. Nat. Rev. Neurol. 2018; 14:168-181.
41. Biessels G J, Despa F. Cognitive decline and dementia in diabetes mellitus: mechanisms and clinical implications. Nat. Rev. Endocrinol. 2018; 14:591-604.
42. Kranenburg G, Van Der Graaf Y, Van Der Leeuw J, et al. The relation between HbA1c and cardiovascular events in patients with type 2 diabetes with and without vascular disease. Diabetes Care. 2015; 38:1930-1936.
43. Lipska K J, Krumholz H M. Is hemoglobin A1c the right outcome for studies of diabetes? JAMA-J. Am. Med. Assoc. 2017; 317:1017-1018.
44. Punthakee Z, Miller M E, Launer L J, et al. Poor cognitive function and risk of severe hypoglycemia in type 2 diabetes: Post hoc epidemiologic analysis of the ACCORD trial. Diabetes Care. 2012;35:787-793.
45. Cukierman-Yaffe T, Gerstein H C, Williamson J D, et al. Relationship between baseline glycemic control and cognitive function in individuals with type 2 diabetes and other cardiovascular risk factors the action to control cardiovascular risk in diabetes-memory in diabetes (ACCORD-MIND) trial. Diabetes Care. 2009; 32:221-226.
46. Launer L J, Miller M E, Williamson J D, et al. Effects of intensive glucose lowering on brain structure and function in people with type 2 diabetes (ACCORD MIND): A randomised open-label substudy. Lancet Neurol. 2011; 10:969-977.
47. Feinkohl I, Price J F, Strachan M W J, Frier B M. The impact of diabetes on cognitive decline: Potential vascular, metabolic, and psychosocial risk factors. Alzheimer's Res. Ther. 2015; 7.
48. Jackson K, Barisone G A, Diaz E, et al. Amylin deposition in the brain: A second amyloid in Alzheimer disease? Ann. Neurol. 2013; 74:517-526.
49. Oskarsson M E, Paulsson J F, Schultz S W, et al. In vivo seeding and cross-seeding of localized amyloidosis: A molecular link between type 2 diabetes and Alzheimer disease. Am. J. Pathol. 2015; 185:834-846.
50. Ly H, Verma N, Wu F, et al. Brain microvascular injury and white matter disease provoked by diabetes-associated hyperamylinemia. Ann. Neurol. 2017; 82:208-222.
51. Schultz N, Byman E, Fex M, Wennstrom M. Amylin alters human brain pericyte viability and NG2 expression. J. Cereb. Blood Flow Metab. 2017; 37:1470-1482.
52. Schultz N, Byman E, Wennström M. Levels of retinal IAPP are altered in Alzheimer's disease patients and correlate with vascular changes and hippocampal IAPP levels. Neurobiol. Aging. 2018; 69:94-101.
53. Martinez-Valbuena I, Valenti-Azcarate R, Amat-Villegas I, et al. Amylin as a potential link between type 2 diabetes and alzheimer disease. Ann. Neurol. 2019; 86:539-551.
54. Westermark P, Andersson A, Westermark G T. Islet amyloid polypeptide, islet amyloid, and diabetes mellitus. Physiol. Rev. 2011; 91:795-826.
55. Banks W A, Kastin A J. Differential permeability of the blood-brain barrier to two pancreatic peptides: Insulin and amylin. Peptides 1998; 19:883-889.
56. Lutz T A. Control of energy homeostasis by amylin. Cell. Mol. Life Sci. 2012; 69:1947-1965.
57. Despa S, Margulies K B, Chen L, et al. Hyperamylinemia contributes to cardiac dysfunction in obesity and diabetes: A study in humans and rats. Circ. Res. 2012; 110:598-608.
58. Liu M, Verma N, Peng X, et al. Hyperamylinemia increases IL-1β synthesis in the heart via peroxidative sarcolemmal injury. Diabetes. 2016; 65:2772-2783.
59. Verma N, Liu M, Ly H, et al. Diabetic microcirculatory disturbances and pathologic erythropoiesis are provoked by deposition of amyloid-forming amylin in red blood cells and capillaries. Kidney Int. 2020; 97:143-155.
60. Srodulski S, Sharma S, Bachstetter A B, et al. Neuroinflammation and neurologic deficits in diabetes linked to brain accumulation of amylin. Mol. Neurodegener. 2014; 9:30.
61. Westermark P, Engstrom U, Johnson K H, et al. Islet amyloid polypeptide: Pinpointing amino acid residues linked to amyloid fibril formation. Proc. Natl. Acad. Sci. U. S. A. 1990; 87:5036-5040.
62. Bartel D P. MicroRNAs: Genomics, Biogenesis, Mechanism, and Function. Cell. 2004; 116:281-297.
63. Wang W X, Rajeev B W, Stromberg A J, et al. The expression of microRNA miR-107 decreases early in Alzheimer's disease and may accelerate disease progression through regulation of β-site amyloid precursor protein-cleaving enzyme 1. J. Neurosci. 2008; 28:1213-1223.
64. Leslie P L, Franklin D A, Liu Y, Zhang Y. p53 Regulates the Expression of LRP1 and Apoptosis through a Stress Intensity-Dependent MicroRNA Feedback Loop. Cell Rep. 2018; 24:1484-1495.
65. Shibata M, Yamada S, Ram Kumar S, et al. Clearance of Alzheimer's amyloid-β1-40 peptide from brain by LDL receptor-related protein-1 at the blood-brain barrier. J. Clin. Invest. 2000; 106:1489-1499.
66. Kang D E, Pietrzik C U, Baum L, et al. Modulation of amyloid β-protein clearance and Alzheimer's disease susceptibility by the LDL receptor-related protein pathway. J. Clin. Invest. 2000; 106:1159-1166.
67. Storck S E, Meister S, Nahrath J, et al. Endothelial LRP1 transports amyloid-β1-42 across the blood-brain barrier. J. Clin. Invest. 2016; 126:123-136.
68. Matveyenko A V., Butler P C. Islet amyloid polypeptide (IAPP) transgenic rodents as models for type 2 diabetes. ILAR J. 2006; 47:225-233.
69. Watson P M D, Paterson J C, Thom G, et al. Modelling the endothelial blood-CNS barriers: A method for the production of robust in vitro models of the rat blood-brain barrier and blood-spinal cord barrier. BMC Neurosci. 2013; 14.
70. Qosa H, LeVine H, Keller J N, Kaddoumi A. Mixed oligomers and monomeric amyloid-β disrupts endothelial cells integrity and reduces monomeric amyloid-β transport across hCMEC/D3 cell line as an in vitro blood-brain barrier model. Biochim. Biophys. Acta-Mol. Basis Dis. 2014; 1842:1806-1815.
71. Verma N, Ly H, Liu M, et al. Intraneuronal Amylin Deposition, Peroxidative Membrane Injury and Increased IL-1β Synthesis in Brains of Alzheimer's Disease Patients with Type-2 Diabetes and in Diabetic HIP Rats. J. Alzheimer's Dis. 2016; 53:259-272.
72. Velmurugan G V., Sundaresan N R, Gupta M P, White C. Defective Nrf2-dependent redox signalling contributes to microvascular dysfunction in type 2 diabetes. Cardiovasc. Res. 2013; 100:143-150.
73. Jimenez S, Navarro V, Moyano J, et al. Disruption of amyloid plaques integrity affects the soluble oligomers content from alzheimer disease brains. PLoS One. 2014; 9.
74. Wang J X, Zhang X J, Li Q, et al. MicroRNA-103/107 regulate programmed necrosis and myocardial ischemia/reperfusion injury through targeting FADD. Circ. Res. 2015; 117:352-363.
75. Lima J F, Cerqueira L, Figueiredo C, et al. Anti-miRNA oligonucleotides: A comprehensive guide for design. RNA Biol. 2018; 15:338-352.
76. Liu C C, Kanekiyo T, Xu H, Bu G. Apolipoprotein e and Alzheimer disease: Risk, mechanisms and therapy. Nat. Rev. Neurol. 2013; 9:106-118.
77. Roberts K F, Elbert D L, Kasten T P, et al. Amyloid-β efflux from the central nervous system into the plasma. Ann. Neurol. 2014; 76:837-844.
78. Montagne A, Zhao Z, Zlokovic B V. Alzheimer's disease: A matter of blood-brain barrier dysfunction? J. Exp. Med. 2017; 214:3151-3169.
79. Trikha S, Jeremic A M. Clustering and internalization of toxic amylin oligomers in pancreatic cells require plasma membrane cholesterol. J. Biol. Chem. 2011; 286:36086-36097.
80. Mendell J T, Olson E N. MicroRNAs in stress signaling and human disease. Cell. 2012; 148:1172-1187.
81. Trajkovski M, Hausser J, Soutschek J, et al. MicroRNAs 103 and 107 regulate insulin sensitivity. Nature. 2011; 474:649-653.
82. Silverberg G D, Messier A A, Miller M C, et al. Amyloid efflux transporter expression at the blood-brain barrier declines in normal aging. J. Neuropathol. Exp. Neurol. 2010; 69:1034-1043.
83. http://www.mirbase.org/cgi-bin/mature.pl?mature_acc=mimat0000824
84. http://www.mirbase.org/cgi-bin/mature.pl?mature_acc=mimat0000826
85. https://horizondiscovery.com/en/products/gene-modulation/knockdown-reagents/controls/PIFs/miRIDIAN-microRNA-Hairpin-Inhibitor-Negative-Control-1?catalognumber=1N-001005-01-05 #description
86.

1. A method of reducing an amount of systemic amylin, comprising: administering to a subject in need thereof an effective amount of a composition that increases epoxyeicosatrienoic acids.

2. The method of claim 1, wherein the composition that increases epoxyeicosatrienoic acids is a soluble epoxide hydrolase inhibitor.

3. The method of claim 2, wherein the soluble epoxide hydrolase inhibitor is 1-(1-propanoylpiperidin-4-yl)-3-[4-(trifluoromethoxy)phenyl]urea (TPPU).

4. The method of claim 3, wherein TPPU is administered orally or intravenously.

5. The method of claim 3, wherein the subject is administered a dose of about 20 micrograms per kilogram TPPU.

6. A method of treating a subject diagnosed with a neurological disease or deficiency, said method comprising: identifying a subject diagnosed with a neurological disease or deficiency and administering an effective amount of a composition that increases epoxyeicosatrienoic acids.

7. The method of claim 6, wherein the composition that increases epoxyeicosatrienoic acids is a soluble epoxide hydrolase inhibitor.

8. The method of claim 7, wherein the soluble epoxide hydrolase inhibitor is 1-(1-propanoylpiperidin-4-yl)-3-[4-(trifluoromethoxy)phenyl]urea (TPPU).

9. The method of claim 8, wherein the subject is administered a dose of about 20 micrograms per kilogram TPPU.

10. The method of claim 8, wherein TPPU is administered orally or intravenously.

11. The method of claim 6, wherein the neurological disease or deficiency is selected from: hypoxic-ischemic brain injury, Alzheimer's Disease, neurological deficits, brain microhemorrhages, or axonal degeneration.

12. A method of treating Alzheimer's Disease comprising: administering an agent that increases LRP1 expression to a subject in need thereof

13. The method of claim 12, wherein the upregulator of LRP1 is a miRNA.

14. The method of claim 13, wherein the administration occurs for at least 12 hours.

15. The method of claim 13, wherein the miRNA is miR-103 (SEQ ID NO: 5).

16. The method of claim 13 , wherein the miRNA is miR-107 (SEQ ID NO: 6).

17. The method of claim 13, wherein the miRNA is administered to the subject at a concentration of about 100 nM.

18. The method of claim 15 and further comprising administering miR-107 (SEQ ID NO: 6) to the subject.


SEQUENCE LISTING (10 sequences)

SEQ ID NO: 1 - DNA, 23 bp, Artificial Sequence
  16S rRNA forward
tcccaatggtgcagaagctatta

SEQ ID NO: 2 - DNA, 23 bp, Artificial Sequence
  16S rRNA reverse
aaggaggctccatttctcttgtc

SEQ ID NO: 3 - DNA, 23 bp, Artificial Sequence
  Beta Actin forward
ctaaaggtgaccaatgctggagg

SEQ ID NO: 4 - DNA, 23 bp, Artificial Sequence
  Beta Actin reverse
tggcatagaggtctttacggatg

SEQ ID NO: 5 - RNA, 23 nt, Artificial Sequence
  miR-103-3p
agcagcauuguacagggcuauga

SEQ ID NO: 6 - RNA, 24 nt, Artificial Sequence
  miR-107-3p
agcuucuuuacaguguugccuugu

SEQ ID NO: 7 - DNA, 20 bp, Artificial Sequence
  LRP1 forward primer
ttgtgctgagccaagacatc

SEQ ID NO: 8 - DNA, 20 bp, Artificial Sequence
  LRP1 reverse primer
ggcgtggaagacatgtaggt

SEQ ID NO: 9 - DNA, 20 bp, Artificial Sequence
  GAPDH forward primer
gctgcgttttacaccctttc

SEQ ID NO: 10 - DNA, 19 bp, Artificial Sequence
  GAPDH reverse primer
gtttgctccaaccaactgc